(19) United States
(12) Patent Application Publication
(10) Pub. No.: US 2015/0198583 A1
(43) Pub. Date: Jul. 16, 2015

(54) METHOD OF BIOMARKER VALIDATION AND TARGET DISCOVER

(71) Applicant: Raj K. BATRA, Beverly Hills, CA (US)

(72) Inventor: Raj K. Batra, Beverly Hills, CA (US)

(21) Appl. No.: 14/414,936

(22) PCT Filed: Jul. 16, 2013
(86) PCT No.: PCT/US13/50712

§ 371 Date: Jan. 15, 2015

Related U.S. Application Data
(60) Provisional application No. 61/672,031, filed on Jul. 16, 2012.

(51) Int. Cl. G01N 33/50

(52) U.S. Cl. CPC G01N 33/5011

(57) ABSTRACT

Disclosed herein are methods of discovering and validating select endophenotypes encompassing tumorigenic cancer stem cells.

### RELATED APPLICATION

This application claims priority from U.S. Provisional Patent Application No. 61/672,031, filed on Jul. 16, 2012, incorporated by reference herein in its entirety.

### BACKGROUND

Lung cancer is the leading cause of cancer and related mortality in the world. The five year survival rate for lung cancer patients is 15% with the application of current diagnostic and treatment strategies. One way to impact the disease mortality is to identify disease biomarkers than can accurately prognosticate (predict tumor cell biology and disease aggression). Such biomarkers would enable improved diagnosis and clinical predictability, and optimized treatment protocols to be provided to patients earlier while minimizing costs and reducing false positives. The best approach to identifying these markers is unclear.

The current approach for identifying biomarkers and candidate targets for detection of cancer is shown in FIGS. 1 and 2. A new approach to uncover the molecular basis of aggressive tumor phenotypes is provided herein and contrasted in these Figures. The proposed approach is applicable to all like models of advanced epithelial tumors (such as Malignant Pleural Effusions arising from breast cancer or Malignant ascites from Ovarian cancers).

### SUMMARY

The disclosure provides a method of assessing the tumorigenic potential of individual tumor populations in a population of cancer cells comprising isolating a sample from the subject comprising the population of cancer cells; separating individual tumor populations in the population of cancer cells from each other based on differential RNA or protein expression; and assessing the tumorigenic potential of the separated individual tumor populations. In one embodiment, the separation is performed using fluorescence activated cell sorting (FACS). In another embodiment, the assessment of tumorigenic potential is performed in vitro. In one specific embodiment, the in vitro assessment of tumorigenic potential is performed using a soft agar test. In another embodiment, the assessment of tumorigenic potential is performed in vivo. In one specific embodiment, the in vivo assessment of tumorigenic potential is performed using immunocompromised mice.

In other embodiments, the method also includes obtaining a single cell suspension of the population of cancer cells after separation and prior assessing tumorigenic potential. In certain embodiments, the population of cancer cells is isolated from a single tumor in the subject. In other embodiments, the tumor population comprises cells that are CD24+, CD44hi, Nkx2.1 (TTF-1)+, SOX-2+, Kras+, p53+, Sca1+, miR34alo or CD133+.

In certain embodiments, the cancer is lung cancer. In certain embodiments, the sample is a malignant pleural effusion (MPE).

The disclosure also provides a method of screening for an effective therapeutic for treatment of a cancer comprising separating individual tumor populations in a population of cancer cells from the cancer to be treated from each other based on differential RNA or protein expression; assessing the tumorigenic potential of the separated individual tumor populations; and screening the individual tumor populations with tumorigenic potential for susceptibility to various cancer therapeutics; wherein, if the screened cancer therapeutic reduces the proliferative capacity of the individual tumor populations with tumorigenic potential then the screened cancer therapeutic is an effective therapeutic for treatment of the cancer in the subject. In one embodiment, the separation is performed using fluorescence activated cell sorting (FACS). In another embodiment, the assessment of tumorigenic potential is performed in vitro. In one specific embodiment, the in vitro assessment of tumorigenic potential is performed using a soft agar test. In another embodiment, the assessment of tumorigenic potential is performed in vivo. In one specific embodiment, the in vivo assessment of tumorigenic potential is performed using immunocompromised mice.

In other embodiments, the method also includes obtaining a single cell suspension of the population of cancer cells after separation and prior assessing tumorigenic potential. In certain embodiments, the population of cancer cells is isolated from a single tumor in the subject. In other embodiments, the tumor population comprises cells that are CD24+, CD44hi, Nkx2.1 (TTF-1)+, SOX-2+, Kras+, p53+, Sca1+, miR34alo or CD133+.

In certain embodiments, the cancer is lung cancer. In certain embodiments, the sample is a malignant pleural effusion (MPE).

The disclosure also provides a method of treating cancer in a subject in need thereof comprising isolating a sample from the subject comprising cancer cells; separating individual tumor populations from each other; assessing the tumorigenic potential of the individual tumor populations; screening the individual tumor populations with high tumorigenic potential for susceptibility to various cancer treatments; and administering to the subject a cancer treatment that one or more of the individual tumor populations with high tumorigenic potential is susceptible to, thereby treating cancer in the subject in need thereof. In one embodiment, the separation is performed using fluorescence activated cell sorting (FACS). In another embodiment, the assessment of tumorigenic potential is performed in vitro. In one specific embodiment, the in vitro assessment of tumorigenic potential is performed using a soft agar test. In another embodiment, the assessment of tumorigenic potential is performed in vivo. In one specific embodiment, the in vivo assessment of tumorigenic potential is performed using immunocompromised mice.

In other embodiments, the method also includes obtaining a single cell suspension of the population of cancer cells after separation and prior assessing tumorigenic potential. In certain embodiments, the population of cancer cells is isolated from a single tumor in the subject. In other embodiments, the tumor population comprises cells that are CD24+, CD44hi, Nkx2.1 (TTF-1)+, SOX-2+, Kras+, p53+, Sca1+, miR34alo or CD133+.

In certain embodiments, the cancer is lung cancer. In certain embodiments, the sample is a malignant pleural effusion (MPE).

The disclosure also provides a method of screening for a biomarker of an individual tumor population with tumorigenic potential comprising separating individual tumor populations in a population of cancer cells from the cancer to be treated from each other based on differential RNA or protein expression; and assessing the tumorigenic potential of the separated individual tumor populations; and wherein, if the individual tumor population has tumorigenic potential then the RNA or protein that was used to separate the individual tumor population based on differential expression is a biomarker of an individual tumor population with tumorigenic potential. In one embodiment, the separation is performed using fluorescence activated cell sorting (FACS). In another embodiment, the assessment of tumorigenic potential is performed in vitro. In one specific embodiment, the in vitro assessment of tumorigenic potential is performed using a soft agar test. In another embodiment, the assessment of tumorigenic potential is performed in vivo. In one specific embodiment, the in vivo assessment of tumorigenic potential is performed using immunocompromised mice.

In other embodiments, the method also includes obtaining a single cell suspension of the population of cancer cells after separation and prior assessing tumorigenic potential. In certain embodiments, the population of cancer cells is isolated from a single tumor in the subject. In other embodiments, the tumor population comprises cells that are CD24+, CD44hi, Nkx2.1 (TTF-1)+, SOX-2+, Kras+, p53+, Sca1+, miR34alo or CD133+.

In certain embodiments, the cancer is lung cancer. In certain embodiments, the sample is a malignant pleural effusion (MPE).

The disclosure also provides a cell line wherein the cell line is derived from lung cancer cells and wherein the cell line over expresses a protein selected from the group consisting of CD24, CD44, Nkx2.1 (TTF-1), SOX-2, Kras, p53, Sca1 and CD133. In one embodiment, the cell line derived from lung cells is selected from the group consisting of NCI-H1373, NCI-H1395, SK-LU-1, HCC2935, HCC4006, HCC827, NCI-H1581, NCI-H23, Human, NCI-H522, NCI-H1435, NCI-H1563, NCI-H1651, NCI-H1734, NCI-H1793, NCI-H1838, NCI-H1975, NCI-H2073, NCI-H2085, NCI-H2228 and NCI-H2342. In another embodiment, the cell line comprises an expression vector wherein the expression vector expresses a protein selected from the group consisting of CD24, CD44, Nkx2.1 (TTF-1), SOX-2, Kras, p53, Sca1 and CD133 in the cell line.

The disclosure also a cell line wherein the cell line is derived from lung cancer cells and wherein the cell line under expresses miR34a. In one embodiment, the cell line derived from lung cells is selected from the group consisting of NCI-H1373, NCI-H1395, SK-LU-1, HCC2935, HCC4006, HCC827, NCI-H1581, NCI-H23, Human, NCI-H522, NCI-H1435, NCI-H1563, NCI-H1651, NCI-H1734, NCI-H1793, NCI-H1838, NCI-H1975, NCI-H2073, NCI-H2085, NCI-H2228 and NCI-H2342. In another embodiment, the cell line comprises a vector wherein the vector knocks down the expression of miR34a in the cell line.

### BRIEF DESCRIPTION OF THE DRAWINGS

FIG. 1 is a schematic contrasting the current paradigm with the methods described herein.

FIG. 2 is a schematic contrasting the current paradigm with the methods described herein.

FIG. 3. Representative example of detection of CD24+ cells in MPE cultures from two different patients. CD24+ gating is shown for tumor cells in the live fraction. Sample 1, left, had 12% CD24+ cells and Sample 2, right, had 98% CD24+ cells. y-axis, CD24; x-axis, CD44, another candidate lung TPC marker. (CD24 is a candidate surface biomarker for aggressive [invasive and metastogenic] cell subsets in lung cancers).

FIG. 4: MPE-processing scheme.

FIG. 5. (A) Representative differences in colony-morphologies of 2 MPE-primary cultures. Photomicrographs of two distinct MPE-primary cultures at various time intervals and magnifications. These data depict intratumoral heterogeneity in terms of varying colony morphologies, all within the same culture condition. (B) MPE derived NSCLC-primary cultures express candidate CSC-molecular markers. Although primary cultures are comprised of mixed cell populations, it is clear that candidate CSC molecular signatures are evident. “CSC” are recognized by qualitative and quantitative differences in the differential expression of surface or intracellular biomarkers in cancer cell subsets. For example, expression of PTEN, Oct4, hTERT, BMI1, SUZ12, and EZH2 mRNA is readily evident in the nucleated cell fraction of MPE (by reverse transcriptase-PCR). (C) Dynamic changes in the candidate CSC-molecular markers in MPE primary cultures suggest CSC-biomarker dynamic interplay with the tumor microenvironment or TME. Depicted are changes in the RTPCR amplicons for Oct4 and PTEN from 3 MPE-cell pellets (607, 307 and 507 MPE; lanes 1, 3 and 5) and the primary cultures in vitro (607,307 and 507 TC; lanes 2, 4 and 6) derived from those same MPE. These data seem to suggest that the primary culture medium (pcm)-TME may promote the survival or selective amplification of biomarkers that characterize CSC-regulatory markers or programs.

FIG. 6. (A) Representative immunohistochemistry (IHC) for candidate CSC marker expression. MPE-tumor specimens were immunolabelled for candidate CSC markers CD44 (top panel), cMET, MDR-1 and ALDH-1 (bottom panel). Arrows/regions highlight cells/clusters that label positive. (B) Live sorting of the CSC•Fraction from MPE-primary cultures: Three different MPE cultures (A, B, C) were collected and labeled for candidate CSC-marker expression (CD44, cMET, CD166). Numbers (upper right corner of FACS-histogram) represent the % of cells that gate at >95% of cells labeled with control antibody. (C) Live sorting of the ALDH positive (CSC) cells from MPE-primary cultures: Shown are FACS-dot histograms of two MPE-primary cultures (A, B). Aldeflour-expression (representing ALDH-activity, abscissa); intensity of CD44 expression (ordinate). The figures on the left panel show control cells in presence of ALDH inhibitor DEAB (+DEAB, negative control); ALDH-positive cells are shown in the right panel in Gate 3, in the absence of DEAB. Note that the ALDH+ cells are also stain intensely for CD44. (D) MPE-primary culture subpopulations can be live-sorted on the basis of cell proliferation: Representative histograms of Day 1 versus Day 9 CFSE-Iabeled primary cultures. Subpopulations that are highly proliferative lose the CFSE label, and those which are quiescent retain the label. In this example, on day 1, 96.14% of the counts lie within the region gated by M1; on day 9, 8.36% of the counts lie within the region gated by M1. These data suggest that if CSC, like tissue progenitors, is a subpopulation in the tumor mix that is characterized by a low turnover rate (thus are label retaining in this assay), they can be separated on the basis of a persistent CFSE label.

FIG. 7. (A) (top): MPE•cultures grown in fetal calf serum (left) are less robust than parallel cultures in autologous medium (pcm; right): Depicted are dot blot histograms of forward versus side scatter of parallel MPE-primary cultures grown in FCS versus PCM. These data suggest that primary cultures in pcm are more robust than parallel primary cultures in FCS. (B) (bottom): Heat-inactivation (left) of MPE fluid adversely affects cell viability in primary cultures: Depicted are Day 24-photomicrographs of MPE-primary cultures grown in parallel in normal (right) versus heat-inactivated (left) MPE-fluid component. These data further suggest that characterizing the proteome and glycome within the pcm will enable us to identify specific environmental factors that both sustain the CSC-phenotypes, or alternatively, repress the expression of biomarkers associated with CSC phenotypes. That the proteome within the pcm can be characterized is evidenced by Table 5, and FIG. 8.

FIG. 8. 2D-PAGE of MPE fluid, Approximately 100˜g protein was loaded onto a 17-cm, 3-10NL IPG strip, and the SDS separation was accomplished with an 8-16% gel. Proteins were stained with Sypro ruby. Many of the darkly stained proteins are proteins commonly found in plasma (e,g., albumin, transferrin, etc). These can be extracted out during analysis to hone in on the uncommon and differentially expressed elements that impact CSC biomarker expression.

FIG. 9. The TME in the malignant pleural effusion (MPE-) model we have developed is comprised of both the extracellular matrix (ECM) components within the stroma or the MPE-tumor clusters, as well as the soluble components within the MPE-fluid fraction. FIG. 9 seems to suggest thatfibrillar collagen is not a major constituent of MPE-tumor cluster stroma: Tumor cell clusters in MPE do not stain positively for fibrillar collagen by Masson's trichrome stain. Compared to control (panel A), fibrillar collagen (brilliant blue in panel A) is not a significant component of the extracellular matrix in the MPE-cytopathology specimens. All photomicrographs are 200×, Bouin's fixed tissues. These results do not exclude the possibility that MPE-tumor stroma is devoid of collagen subtypes that comprise basement membranes, or that ALL MPE tumor clusters are devoid of fibrillar collagen.

FIG. 10: CS•PGs are a component of MPEs (see Batra et al; JBC 1997). Dot blot immunoassay reveals Chondroitin-4-sulfate (2B6 Ab), c-6-sulfate (3B3 antibody), and native CS-epitopes (7D4 antibody) in MPE-supernatants. 100 ul of seven (1-7) serially diluted effusion specimens (1:1000, 1:5000, 1:10,000, and 1:50,000 in Tris-salt buffer) were assayed for the presence of C-4-S and C-6-S using bovine nasal cartilage core (at concentrations of 50 ng/ml, 10 ng/ml, 5 ng/ml, and 1 ng/ml) as the control (C) antigen. Samples were applied to nitrocellulose, blocked, then pre-treated with chondroitinase ABC. Similarly, 100 ul of six serially diluted effusion specimens (1:1000; 1:2500; 1:10.000; 1:25,000) were assayed for the presence of native CS epitope (7D4 antibody) without chondroitinase pretreatment and using digested shark cartilage (50 ng/ml, 10 ng/ml, 5 ng/ml, 1 ng/ml) as the control (C) epitope. The primary murine antibodies (diluted 1:1000 in 1% BSA) were recognized by anti-mouse IgG (diluted 1:7500 in 1% BSA) conjugated to alkaline phosphatase (AP), detected following exposure to the AP substrate. The reference cited above characterizes the scope of glycosaminoglycans and proteoglycans present in the MPE milieu. The GAGs and PGs include sulfated and non-sulfated species, of highly variable molecular weights (see Batra R K et al; JBC 1997).

FIG. 11: Representative Photomicrographs of the E2F regulated expression of PCNA in a subpopulation of cells within tumor cell clusters. This observation simply confirms that cells within the tumor clusters within MPE are not senescent because they are actively proliferating. PCNA was immunoabeled using PC10 (Dako, Carpinteria, Calif.), and detected using the ABC-Vector Red kit.

FIG. 12: Growth Kinetics of MPE Cell Preparations injected subcutaneously into the flank region of nu/nu mice. 1×10⁵ to 1.1×10⁷ cells were into the post flank region of nu/nu mice to generate tumors. Tumor dimensions were estimated based on bisecting diameters measured with a caliper, and the tumor volume was approximated using the formula 0.4 (ab2) where a is the long measured axis of the tumor and b is the short measured axis. Depicted are the individual (when a lone mouse was injected with tumor) or mean (when two mice were injected with cells) tumor volumes based on the caliper measurements in these pilot studies. These data suggest that it is difficult to establish a baseline threshold for numbers of tumor cells in MPE that will reliably result in tumor formation in immunocompromised mice. Contrast these data with that of FIG. 15. There, tumors were reliably established from cells that were fractionated from MPE-primary cultures on the basis of differential surface expression of a biomarker (CD44). Collectively, these data indicate that primary cultures enable us to more readily sort out aggressive tumor cells from non-aggressive tumor cells (which is less practical with freshly isolated MPE-tumors that require tissue digestion). Importantly, our approach and techniques differ from those of other competitors because our primary cultures are established in an “autologous” TME (MPE cells and fluid components from the same subject whose tumor is primarily cultured).

FIG. 13: H&E staining of representative cytopathology from MPE (1A, 1B, 1C) and histopathology of extirpated tumors (2A. 2B) they engendered tumors upon in vivo transplantation. [1A/2A are from sample 407, and 1B/2B are from sample 307, and 1C is from 206, Table 6]. These data suggest that xeno-engrafted tumors that are generated from sorted MPE-primary cultures histologically resemble those of the subject from whom the MPE was extracted.

FIG. 14: CD44^{hi} MPE-tumor cell population is more tumorigenic than the CD44^{lo} cells: A representative MPE-tumor primary culture is segregated on the basis of high versus low CD44 expression. Isogenic cell populations are injected in equivalent numbers (30,000 cells) in the left (CD44^{hi}) or right (CD44^{lo}) subcutaneous flank region of a SCID mouse. The CD44^{hi} cell population generates a tumor, whereas the CD44^{lo} does not. Thus, the CD44^{hi} population is enriched for cells that mediate tumorigenic potential. The demonstration that 300 CD44hi cells can generate tumors in this example indicates that the CD44hi fraction contains “cancer stem cells”, or CSC (see FIG. 15). The demonstration that CD44lo or mixed (unsorted) cell populations do NOT generate reliable tumors suggest that some cell-cell interactions within tumor populations may actually deter tumor formation and growth by highly aggressive CSC.

FIG. 15. (A) CD44^{hi} subpopulation of an MPE-culture displays “stemness” tumorigenic properties in NOD/SCID IL2γR^{null} mice: MPE-primary culture is separated on the basis of high versus low CD44 expression. Limiting dilutions of cells are implanted in the flanks of NOD/SCID IL2γR^{null} mice, and tumor volume monitored over time. In the CD44^{hi} subset, tumorigenesis is evident at a dose of 300 cells (in 1 of 3 animals), whereas 30,000 CD44^{lo} cells from the same population are unable to form tumors. (B) Histopathology of the CD44^{hi} tumor displays morphologic and antigenic (CD44-immunolabeling) heterogeneity. (C) CD44^{hi} tumor cells consistently display enhanced soft agar colony forming potential. Depicted are the relative differences in colony forming units (mean+1−standard deviation, n=3 replicates) between the CD44^{hi} versus CD44^{lo} cells (at 3 weeks after 8000 cells seeded/well) from 3 different MPE biospecimens. (D) CD44^{hi} tumor cells display differences in colony size and morphology from CD44^{lo} tumor cells. Depicted are representative photomicrographs (20×) of colony size and morphology differences between the CD44^{hi} versus CD44^{lo} cells (at 3 weeks after 8000 cells seeded/well) from an MPE biospecimen.

FIG. 16. (A) Discrete foci (estimated 26% of total area) within tumor clusters within MPE are CD44+. After quenching (PBS+2% hydrogen peroxide), prepared specimens were blocked with normal mouse serum for 30 min at RT. Primary antibody (mouse anti-CD 44, Abcam) was applied (1 hr, RT), washed, and slides were incubated with anti-mouse HRP conjugate (Santa Cruz, 30 min RT). Tissue sections were rinsed with PBS and developed using the DAB substrate kit (Vector laboratories). The stained sections were observed under the microscope (Olympus BX-61, 20× under phase illumination), and the captured images were analyzed using the Openlab software. The positively stained cell area was estimated using Image Pro Plus software. Cell clusters were defined manually using phase images and the irregular AOI tool, and the resultant groups were segmented based on an empirically determined positive staining threshold. Percent of positively stained cells was estimated, based on the fractional area of staining within the total cluster area. (B) Fractional expression of CD44 changes as cells are passages in culture: FACS analysis of primary cultured MPE-cells (10⁶ cells were labeled with 1˜g PE-mouse Anti-Human CD44, BD Pharmingen, 45 minutes, washed, and spot fixed with 2% paraformaldehyde solution, and washed for standard multichannel FACS data acquisition with a Becton Dickinson FACSCalibur cytometer (Jonsson Comprehensive Cancer Center, UCLA, CA). Data was analyzed using FCS Express analysis software (De Novo Software, Ontario, Canada). (C) Differential labeling (4c3 monoclonal Ab) suggests distinct zones of CS-PG distribution in the pericellular matrix of MPE-tumor clusters (specimen 407). Photomicrograph depicts representative differential labeling of the cell surface and pericellular matrix by the 4C3 murine monoclonal (lgM, κ-isotype) in MPE-tumor clusters (from sample 407). 4C3 recognizes distinct “native” CS sulfation motifs, typically associated with perlecan, and less likely versican or CS-substituted decorin matrix PGs. (D) Possible changes in the expression of programs important for SC-phenotype as cells are passaged in culture: Depicted are the relative changes in the RT-PCR amplicons for Oct4 and PTEN from 3 primary isolates (607, 307 and 507 MPE; lanes 1, 3 and 5) and the primary cultures in vitro (607,307 and 507 TC; lanes 2, 4 and 6). RNA was extracted from MPE clusters and the passaged cells using Triozol reagent and Fast Track 2.0 mRNA isolation kit (Invitrogen Inc., Carlsbad, Calif.). 500 ng of the mRNA was reverse transcribed using the RT kit and cDNA prepared (Invitrogen, Carlsbad, Calif.). Two ul aliquot of the synthesized cDNA was used for the amplification of PTEN and Oct4 genes by PCR Amplifications were carried out in a total volume of 20 ul for 35 cycles of 1 minute at 94° C. and 1 minute at 57.5° C. PCR products were separated on 8% TBE gels, EtBr-stained, and images captured using the Kodak 1 D software. These data seem to suggest the presence of microdomains within individual tumor clusters that harbor discrete niches for CSC. The stromal components of those niches may exclude biologically active soluble factors in MPE-fluid to keep the CSC-niche quiescent. Breakdown of those niches, as occurs during the evolution of primary cultures (see Basak et al, PLoS ONE, 2009) possibly expose the CSC to environmental activators that contribute to cell cycle progression/biological aggression of CSC.

FIG. 17. (A) Nkx2.1 is expressed in MPE. (B) The FUGW lentiviral vector (LV) prototype efficiently transduces model lung cancer cells. (C) Transduction efficiency of MPE-primary cultures: A MPE-primary culture is transduced at various log-dilutions of the control (CMV driving eGFP; FUGW construct) VSV-G pseudotyped LV. The concentrated vector stock is 10e8 egfp+unit1 ml, determined by transducing 293T cells. These data provide proof-of-concept that subsets of tumor cells in the MPE-populations express primordial (developmental) transcription factors (e.g.: Nkx2.1, SOX2, Grhl2 etc). If cancer stem cells are “arrested” in a developmentally primitive state, then cells expressing these developmental factors may represent CSC. We can isolate these subsets by “transcriptional sorting” techniques. For example, we can generate lentiviral vectors that encode the tandem repeats of the response elements of these (and other) primordial transcription factors to drive the expression of a fluorescent reporter gene. Then, we can live sort out the cell fraction that highly expresses the primordial transcription factors, and validate them for “CSC” behavioral properties.

FIG. 18. (A) Metaphase spreads were probed with Fluorescent in situ hybridization. The orange probe detects a region on chromosome 1p36 that includes the miR34a locus; the control green probe recognized a region on 1q25. (B) FISH analysis of representative MPE sample reveals an abnormal hyperdiploid karyotype, with 4 copies of Chromosome 1. 3 of these have with intact 1p/1q regions; 1 of the chromosomes has a deleted 1p region. (C) FISH analysis of representative MPE sample reveals an abnormal hyperdiploid karyotype, with 2 copies of 1p and 6 copies of 1q. These findings suggest a 1p deletion. One candidate gene that may impact CSC phenotypes in this region is the miR34a gene.

FIG. 19: Tumorigenic properties of the CD44^{hi} subset in lung cancer is variably associated with decreased miR34a expression. Indexed to the expression of small nucleolar RNA-48, depicted are relative expression of miR34a in unsorted cell, as well as CD44^{hi} and CD44^{lo} subsets in two MPE-primary tumor specimens, and a model lung adenoCa cell line (NCI H2122). Note that the fibroblast control (as well as MPE tumor 1) does not display such differences in miR34a between the CD44^{hi} and CD44^{lo} subsets. The CD44hi cells in the H2122 cell line, and in the MPE tumor 2 primary culture, display lower miR34a expression than CD44lo cells. These data suggest that abnormal expression of miRNAs is one mechanism by which tumor cells may become dysregulated to participate in aggressive (e.g., tumorigenic) behaviors.

FIG. 20. (A) Exogenous delivery of miR34a into CD44^{hi} cells inhibits soft agar colony formation. Depicted are the relative differences in colony forming units (mean+/−standard deviation, n=3 replicates). Following live-sorting, CD44^{hi} cells were transfected (50 pmol) with miR34a versus control, and 2500 cells were seeded in soft agar colony assays. Colonies were counted 3 weeks later. (B) Exogenous delivery of anti-miR34a into CD44^{lo} cells enhances soft agar colony formation. Depicted are the relative differences in colony forming units (mean+/−standard deviation, n=3 replicates). Following live-sorting, CD44^{lo} cells were transfected (50 pmol) with the anti-miR34a versus control miR and plated in soft agar colony assays. Colonies were counted 3 weeks after plating 8,000 cells.

FIG. 21 shows morphologically variant cells in MPE samples and absence of morphological changes between CD44^{hi} and CD44^{lo} cells. The tumor cells from M-1, M-2 and M-3. (A) 100× (2-3 weeks, 100×). (B) 400× (2-3 weeks). (C) Later stages of culture 100× (6-10 weeks). (D) CD44-FACS expression pattern and MFI. (E) Sorting of CD44^{hi} and CD44^{lo} cells (5-10%). The sorted cells CD44^{hi} and CD44^{lo} were washed and plated out in PCM for 2-3 days to evaluate their morphological differences. (F) Sorted CD44^{hi} cells and sorted (G) CD44^{lo} cells were (100×). The purity of the CD44hi and CD44^{lo} cells were ≧98%, as revealed by post sort analysis (data not shown).

FIG. 22 shows higher clonal efficiency and colony forming potential of CD44^{hi} cells and their CSC molecular markers expression in comparison to CD44^{lo} cells. The sorted cells CD44^{hi} and CD44^{lo} from MPE samples were analyzed in triplicates for their (A) clonal efficiency (Sample M-1: colonies CD44^{hi}=35.8 (SD=5.04) vs CD44^{lo}=21.7 (SD=6.2) (P=0.03); Sample M-2 colonies CD44^{hi}=59.8 (SD=3.2) vs CD44^{lo}=40.6 (SD=4.1) (P=0.003); Sample M-3 colonies CD44^{hi}=53.4 (SD=5.3) vs CD44^{lo}=33.9 (SD=3.6) (P=0.006)); The mean effect of CD44^{hi} versus CD44^{lo} is 17.6 (95% CI: 8.31, 26.89: p=0.015). (B) colony forming ability in soft agar. (Sample M-1: colonies CD44^{hi}=16.6 (SD=1.1) vs CD44^{lo}=8 (SD=1.1) (P=0.0006); Sample M-2: colonies CD44^{hi}=27 (SD=7) vs CD44^{lo}=12 (SD=3) (P=0.02); Sample M-3: colonies CD44^{hi}=24.3 (SD=6.1) vs CD44^{lo}=12.6 (SD=2.5) (P=0.03)). The mean effect of CD44^{hi} versus CD44^{lo} is 11.8 (95% CI: 3.41, 20.14; P=0.026). (C) Soft agar colonies derived from CD44^{hi} cells (100×) and (D) CD44^{lo} cells (100×). Columns, mean from three independent experiment; SD, *, P<0.001, compared with the CD44^{lo} groups, (student's t test). (D) Expression profile of BMI-1, hTERT, SUZ12, EZH2 and OCT-4 in sorted CD44^{hi} and CD44^{lo} cells were analyzed by reverse transcriptase-PCR. In sample M-3 only BMI-1 is expressed at high level in CD44^{hi} population than the CD44^{lo} cell population. In sample M-2 there slight higher expression of hTERT in CD44^{hi} cells than CD44^{lo} cells. The CD44^{hi} population of sample M-1 expressed high level of BMI-1, hTERT, SUZ12, EZH2 and OCT-4, than CD44^{lo} population.

FIG. 23 shows tumorigenicity of CD44^{hi} population from primary tumor cells in NOD/SCID (IL2rγ^{null}) mice. (A) Tumorigenicity and latency period of CD44^{hi} cells (M-1) injected with at 30,000; 3,000 and 300 cells. Mice injected with CD44^{hi} (right flank) formed tumors and CD44^{lo} cells did not form tumor (left flank). The numbers (1/3, 2/3 or 3/3) represent number of animals with tumor/group at particular time point of measurement. Time period of days after tumor implantation is expressed along X axis and tumor growth volume is expressed as mm³ along Y axis. Unsorted parental primary tumors implanted with 500,000 cell/mice did not show any tumor growth even after 3 months of tumor cell implantation (data not shown) (B) Mice bearing tumors on the right flank injected with CD44^{hi} cells and no tumor was detected at the left flank injected with CD44^{lo} cells (C) Resected tumors formed by CD44^{hi} cells in mice. (D) FACS analysis of single cells obtained from mouse tumors derived from CD44^{hi} cells of M-1 MPE sample. The tumor cells show expression of CD44, cMET, uPAR and CD166 markers. (E) Tumorigenicity and latency period of CD44^{hi} and CD44^{lo} cells of samples M-1 and M-2 in NOD/SCID (IL2rγ^{null}) mice.

FIG. 24 shows immunohistological study of tumors generated by CD44^{hi} cell population in mouse, human squamous cell carcinomas (SCCs), human alveolar and human bronchiolar tissues. The photomicrograph A, B, C and D represent the tumors derived from Sample M-1 and E, F, G and H represent tumor derived from sample M-2 in NOD/SCID (IL2rγ^{null}) mice. The following stains are represented: H&E staining (A and E), immunohistochemistry for CD44 expression (B and F), ALDH expression pattern (C and G) and the immunohistochemistry for dual CD44 and ALDH staining (D and H). Representative human lung squamous cell carcinoma (SCC) tissue samples (I, J, K and L, M, and N) were stained by H&E (I), CD44 (J and L), ALDH (K and M) and immunohistochemical staining for dual markers CD44 and ALDH (N). Human alveolar tissue sections were stained for CD44 (O), ALDH (P) and dual markers CD44 and ALDH (Q). Human-bronchiolar tissue sample was stained for CD44 (R), ALDH (S) and dual markers CD44 and ALDH (T).

FIG. 25 shows karyotype and Fluorescent In Situ Hybridization (FISH) analysis of MPE derived tumor cells: (A) Dual color FISH analysis was done using 1p36 and 1q25 (control) probes. Representative position of the probe 1p36 (orange) and 1q25 (green) on chromosome 1. (B) Sample M-1: Abnormal hyperdiploid karyotype (83 chromosomes) and (C) with 3 copies of chromosome 1 (↑) but have 2 copies (Δ) of rearranged 1p and 1q. (D) Sample M-2: Abnormal hyperdiploid karyotype (67 chromosomes) and (E) with 4 Chromosome is (↑) (3 with intact 1p/1q and 1 with 1p deletion (Δ)). (F) Sample M-3: Abnormal hyperdiploid karyotype (74 chromosomes) and (G) with 2 copies of 1p (Δ) and 6 (↑) copies of 1q (consistent with 1p deletion). (H) Sample NCI-H2122 Abnormal karyotype (58 chromosomes) and (I) with 2 copies (↑ and Δ) of 1p/1q but one 1p is rearranged with additional material of unknown origin at 1p terminal region (Δ). (J) Normal deployed human fibroblast cell line GM 05399 control with two copies of 1p/1q (↑).

FIG. 26 shows expression of miR-34a in CD44^{hi} and CD44^{lo} cells evaluated by RT-qPCR and exogenous delivery of miR-34a into CD44^{hi} cells inhibits colony formation and anti-miR-34a into CD44^{lo} cells increases colony formation: (Δ) miR-34a expression in unsorted, CD44^{hi} and CD44^{lo} cells of two primary samples (M-1 and M-2), established NSCLC cell line NCI-H-2122 and normal human fibroblast cell line GM 05399. The miR-34a expression has been normalized with RNU48. (B) Sorted CD44^{hi} cells (samples M-1 and M-2) when transduced with miR-34a show decreased colony forming efficiency in comparison to miR-control transduced CD44^{hi} tumor cells; (Sample M-1: miR control=131.6 (SD=30.5), +miR-34a=7 (SD=2.6) and P=0.002; Sample M-2: miR control=75 (SD=19.6), +miR-34a=23 (SD=5.5) and P=0.01); The mean effect with miR-34a versus miR-control on CD44^{hi} cells is −88.3 (95% CI: −288.12, 111.45; P=0.112) (C) Sorted CD44^{hi} tumor cells transduced with miR-34a exhibit small colony size (bottom panel) than miR-control transduced CD44^{hi} tumor cells (upper panel); (D) The CD44^{lo} cells from Sample M-1 and M-2 transduced with anti-miR34a show increased colony forming efficiency than tumor cells transduced with miR-control; (Sample M-1: miR control=21 (SD=4.5), +anti-miR-34a=33 (SD=5.2) and P=0.04; Sample M-2: miR control=24.6 (SD=4.1), +anti-miR-34a=39.3 (SD=5.1) and P=0.018); The mean effect with anti-miR-34a versus miR-control on CD44^{lo} cells is −13.3 (95% CI: −20.43, 47.10; P=0.125). (E) Sorted CD44^{lo} cells transduced with anti-miR-34a exhibit bigger colony size (bottom panel) than miR-control transduced tumor cells (upper panel).

FIG. 27 shows cell cycle parameters of CD44^{hi} and CD44^{lo} cell populations derived from primary cultures of MPE tumors. Representative FACS analysis of CD44 and PI staining (i and ii) of three samples (A) M-1, (B) M-2 and (C) M-3. The samples were evaluated for their CD44^{hi} (iii) and CD44^{lo} (iv) and total cells (v, no gate) cell cycle and their G1, S and G2 phase analysis. (D) The results indicate that gated CD44^{hi} population express high level of S and G2 phase (Sample M-1: S/G2: 15.75/72.61; Sample M-2: S/G2: 12.03/32.19; Sample M-3: S/G2: 6.25/15.17) than the gated CD44^{lo} population (Sample M-1: S/G2: 4.36/1.81; Sample M-2: S/G2: 5.18/7.14; Sample M-3: S/G2: 3.37/3.32) respectively.

FIG. 28 shows a representative screenshot of differences in GLDC-gene methylation. These data suggest that aggressive (tumorigenic) lung cancer cell subsets in individual tumors may be discriminated from non-aggressive (non-tumorigenic) subsets by differences in GLDC gene methylation or mRNA/protein expression. This observation is substantiated by the a 2012 report (Zhang et al, Cell 2012) that GLDC is differentially expressed in lung cancer stem cells, and which provides some evidence for validating this biomarker as a metabolic target in lung cancer.

FIGS. 29A, 29B and 29C are light micrographs showing Sox2 staining in primary cells (29A and B and a cell line (29C). These data substantiate the proof-of-concept presented in FIG. 17.

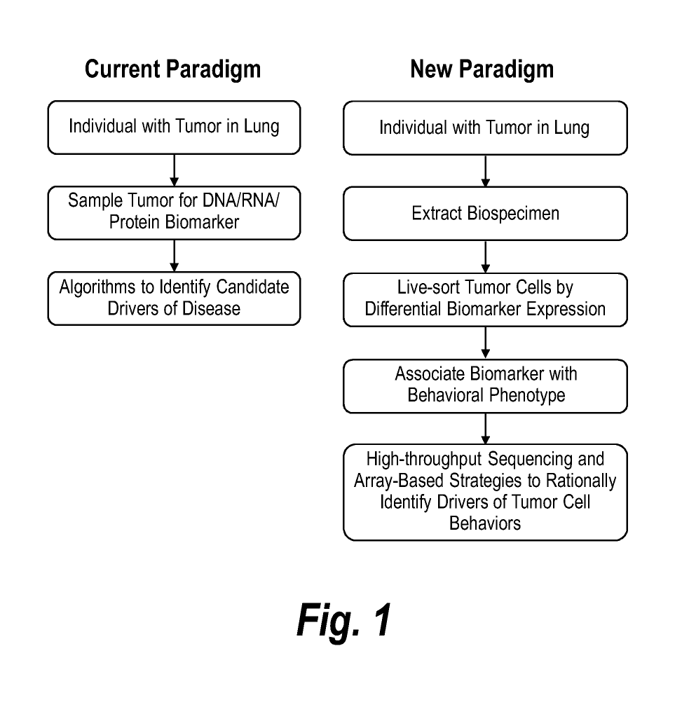

### DETAILED DESCRIPTION

Within an individual tumor population, some tumor cell subsets are inherently more capable than others in forming tumors, to metastasize, and/or to resist therapy. Collectively, these properties have been attributed to “cancer stem cells” (CSCs). Described herein are methods to extract candidate CSCs from clinical biospecimens. Further described herein is validation that cell subsets live-sorted on the basis of expressed CSC-biomarkers are reliably more tumorigenic than other tumor cells from the same. Also described herein are key candidate targets that have emerged from analyses of genetic/epigenetic signatures that distinguish tumorigenic from non-tumorigenic subsets in the same tumor population. These data indicate that the approach we have developed has the potential to yield both diagnostic/prognostic signatures of aggressive diseased cell subsets in lung cancer, as well as for the efficient and agnostic identification of candidate molecular targets to ablate the disease.

As described, the lethal properties (tumor formation, metastasis, drug resistance) of solitary tumors are attributed to aggressive “cancer stem cells” (CSC), which are uniquely hyperplastic, and which possess the requisite genetic/epigenetic repertoire to mediate lethal behaviors. Whereas an individual tumor is comprised of many distinct cancer cell phenotypes, many/most of these may not be relevant to prognosis.

Cellular and molecular heterogeneity is evident throughout the course of lung cancer pathogenesis. Intratumoral heterogeneity is a key cause of therapeutic resistance in lung cancer. Because of intratumoral heterogeneity, an individual tumor is typically a “combination of diseases”, or comprised of functionally distinct cancer cells due to underlying genetic, epigenetic or contextual (microenvironment-associated) differences.

Thus, the lung cancer of an individual patient is a mixture of cancer cells with varying properties. The term isogenic is used, herein, to describe tumor cells collected from a single individual. The term “endophenotypes” is used herein to describe isogenic cells that differ in behavior. The disclosure provides several methods to rationally segregate isogenic lung cancer cell populations to isolate endophenotypes in bioassays. Because the derivative subpopulations are isogenic, endophenotypes can be directly compared (using high throughput array-based and next generation sequencing (NGS) techniques) to efficiently uncover the genetic/epigenetic bases for behavioral properties. Thus, the molecular underpinnings of a particular phenotype can be rationally discovered, and combinations of emerging “targeted therapies” can be rationally applied.

The disclosure provides a method of determining biomarkers that represent populations comprising highly plastic tumorigenic “cancer stem cells”. In certain embodiments, these methods include isolating cancer cells from a subject for analysis. These cancer cells can be from a tumor or any other biological sample from which cancer cells can be isolated. Biological samples include organs and tissues including blood (serum, red blood cells, white blood cells and/or platelets), lung, heart, skeletal muscle, smooth muscle, gastrointestinal tract (esophagus, stomach, small intestine, large intestine and/or rectum), lymph, eyes, nose, throat, mouth, brain spinal cord, skin, mucous membranes, testicles, penis, bladder, pancreas, liver, gall bladder, kidney, bone and connective tissue. In certain specific embodiments, the cancer is isolated form lung tissue. Optionally, the lung cancer is isolated from malignant pleural effusions (MPE). The lung cancer can be selected from squamous cell carcinoma, adenocarcinoma, large cell carcinoma and small cell carcinoma.

In certain embodiments, subjects are mammalian subjects. Mammalian subjects include rodents, livestock, primates or pets. Rodents include rats, hamsters, gerbils, mice or rabbits. Livestock include sheep, goats, llamas, camels, cattle, or buffalo. Primates include monkeys, apes or humans. Pets include dogs or cats. In certain preferred embodiments, the mammalian subjects are humans.

In certain embodiments, after the cancer cells are isolated for analysis, the cells are made into a single cell suspension, i.e. the cells are made into a suspension where substantially all of the cells are suspended in a fluid where most of the cells are not adhering to other cells. A single cell suspension can be made by exposing the cells to a dissociation enzyme. Dissociation enzymes are well known in the art and include trypsin, hyaluronidase, papain, elastase, DNase, protease type XIV or collagenase. After the dissociation of the cells, they can be spun down and resuspended in medium. In other embodiments, the cells are already in suspension or for other reasons, the cells do not need to be made into a single cell suspension.

In certain embodiments, tumor cell preparations lose viability when they undergo digestion. In these embodiments, methods are used to enhance viability of the tumor cell populations. For example, for cultures isolated from MPE, primary cultures can be formed over time (3-5 weeks) in the MPE-fluid component that is derived from the patient. This fluid component can act as an autologous tumor microenvironment (TME). That primary culture can then be digested to generate a non-adherent tumor cell suspension that can readily undergo FACS sorting. In certain embodiments, the digestion of adherent cells in primary cultures is performed with trypsin.

The cancer cells are then fractionated based on differential expression of various proteins or RNAs. The proteins or RNAs can be any that affect the tumorigenicity of cancer cells. RNAs can be mRNAs that express proteins that affect the tumorigenicty of cancer cells or miRNAs and/or siRNAs that reduce the expression of other RNAs including other mRNAs. Several differentially expressed proteins can be used for fractionation. For example, cell surface proteins that putatively predict for cells with aggressive properties, for example, CD24, CD44, CD166, cMet, uPAR, MDR1 and CD133 enable sorting of candidate aggressive cell subsets. Similarly, intracellular signaling proteins, and transcription factors including mutated K-ras, mutated or lost p53, Nkx2.1 (TTF-1), SOX-2, and “embryonal” markers such as Nanog and Oct3/4] also enable extraction of candidate cancer cell subsets with more aggressive features. In a like manner, intracellular metabolic markers (such as Aldehyde dehydrogenase enzymes that modulate xenobiotic metabolism, or Glycine decarboxylase that helps shuttle single carbon metabolites to nucleotide synthetic pathways) also enable differential sorting of more aggressive cancer cell subsets. Finally, RNA species, especially cells that exhibit differential expression of miRNAs (e.g.: miR34a) enable differential sorting of more aggressive cancer cell subsets within individual tumors.

Throughout the disclosure, cells are referred to as being positive (+) or negative (−) for the presence of a protein or RNA or a cell being high expressing (^{hi}) or low expressing (^{lo}) for a certain protein or RNA. According to certain embodiments, a cell that is + for a protein or RNA has detectable amounts of the protein or RNA while a compared population has no detectable amounts of the protein or RNA. According to other embodiments, a cell that is + for a protein or RNA has greater than 50% more of the protein or RNA than population that is − for the protein or RNA. According to other embodiments, a cell that is + for a protein or RNA has greater than 75, 100, 150, 200, 250, 300, 350, 400, 450 or 500% more of the protein or RNA than population that is—for the protein or RNA. According to certain embodiments, a cell that is defined as ^{hi} for a protein or RNA has more of the protein or RNA than a cell that is defined as ^{lo} for a protein or RNA. According to other embodiments, a cell that is defined as ^{hi} for a protein or RNA has greater than 5, 10, 15, 20, 25, 30, 35, 40, 45, 50, 55, 60, 65, 70, 75, 80, 85, 90, 95 or 100% more of the protein or RNA than a cell that is defined as ^{lo} for a protein or RNA.

Fractionation can be performed using any method known in the art. In certain embodiments, fractionation is performed using fluorescence activated cell sorting (FACS). Antibodies specific for a certain protein can be fluorescently labeled and sorted according to the association of the fluorescent signal with cells. However, any antibody isolation technique that isolates the cells intact could be used. Many bead based technologies including use of magnetic beads or substrate bound beads can be used to isolate cells associated with antibodies. Further, cancer cells can be transfected with reporter constructs that can be used to measure the expression of RNA. These RNAs can be mRNAs, miRNAs, or shRNAs. Expression of many mRNAs can be correlated with protein expression of their gene products. The reporter constructs can provide fluorescent signals that can be detected using FACS or other means. Cells can also be differentially sorted on the basis of differences in metabolism. For example, aggressive cancer cell subsets that express high levels of Aldehyde dehydrogenase can be extracted from mixed populations by the use of a fluorescent marker (Aldefluor™) that is activated when a substrate is exposed to this family of enzymes.

Cancer cells that are fractionated form sorted populations are then assessed for tumorigenicity potential. Tumorigenicity can be measured using any method known in the art. Methods include injecting the cells into immunocompromised mammal models. These models include immunocompromised mouse models. The mouse models include SCID mice, C57BL/6J mice and athymic or nude mice. Tumorigenicity can also be measured using in vitro models. In vitro models include soft agar assay, SHE cell transformation assay or colony/focus formation assays.

When a fractionated cell population has higher tumorigenicity than the parent cell populations and/or the cell population that it is fractionated away from, the fractionated cell population is more likely to include a tumorigenic cancer stem cell. That is, the fractionation of the cell population shows that the protein or RNA that was the basis for the fractionation is likely to be more highly expressed in tumorigenic cancer stem cells than in the rest of the cancer cell population. The isolation of a tumorigenic (or metastogenic) cell population away from the bulk tumor enables one to capture the nucleic acids and proteins that are not only more commonly associated with tumorigenic and metastatic properties (biomarkers of disease), but also enables the enrichment of nucleic acids and proteins that are responsible for mediating those aggressive behavioral properties (targets of disease). By capturing the signatures that are associated with the growth and metastasis of tumors allows one to diagnose and prognosticate aggressive disease features; by identifying and validating targets that are responsible for mediating aggressive features enables one to effectively treat the disease. Thus, this fractionated cell population can be focused on for the development of therapeutics for cancer treatments. Also, this cell population can be used to determine accurate markers associated with a specific type of cancer and in certain embodiments, associate that specific type of cancer with an effective therapeutic.

In certain embodiments, assessment of tumorigenicity means that a given cell population promotes metastasis in a model of tumor cell invasion, extravasation into lymphatics or blood stream, and metastatic seeding followed by growth in a distinct organ that is different from the organ of tumor origin (e.g.: lung). In other embodiments, assessment of tumorigenicity means that a given cell population provides greater metastasis than a control cell population, or that a particular biomarker mediates organ-specific metastases greater than isogenic counterparts. For example, specific lung cancer cell subsets may demonstrate greater proclivity than others to mediate seeding and growth (metastases) of cancer cells within the brain, liver, bone, adrenal or lung tissues. Thus, a fractionated cell population may be assessed as being more metastogenic if it promotes metastasis more than its parent population and/or the population of cells it is sorted from. In these embodiments, the increase in promotion of metastasis can be greater than 10, 20, 30, 40, 50, 60, 70, 80, 90 or 100%.

Development of Cancer Therapeutics

The disclosure provides methods of developing cancer therapeutics. A tumor cell population isolated from a specific tissue or from a specific tumor in a subject is not made up of a homogenous population of cells. Tumors tend to be varying populations of cells that have evolved with time. Certain subpopulations of cells within the tumor are more active contributors in the growth of the tumor and/or metastasis of cancer cells to other sites in the subject.

In certain embodiments, an effective method for screening for effective drugs is isolating this tumorigenic cancer stem cell population from multiple subjects and then, using high throughput array based and next generation sequencing (NGS) strategies, to generate common signatures of “cancer stem cell” aggressiveness from these subjects. These common signatures represent candidate common targets that can be validated by more specific molecular screening, and then by molecular or pharmacological approaches to ablate the phenotypic (behavioral) effects of the targets. In this manner, not only are novel targets discovered from a phenotype-based discovery effort, but rational combination therapy strategies are derived. Administering agents or combinations of agents in subjects who harbor similar biomarker-based cancer stem cells enables establishment of safety and efficacy profiles of single and/or combinatorial targeted therapeutic strategies.

According to certain embodiments, when a tumorigenic cancer stem cell population is generated, it is exposed to a number of anti-cancer therapeutics to find one or more therapeutics that are effective in reducing the proliferation of the selected tumorigenic cancer stem cell population.

In certain embodiments, multiple tumorigenic cancer stem cell populations can be selected from a single population of cancer cells or a single tumor. This can be done according to several methods.

In one embodiment, a population of cancer cells is fractionated repeatedly in parallel with proteins or RNAs. In this embodiment, a population of cancer cells from a subject is split into two or more populations. According to certain embodiments, the population of cancer cells is split into 2, 3, 4, 5, 6, 7, 8, 9, 10 or more populations. According to this embodiment, each of the aliquots of the population of cancer cells is fractionated using a different protein or RNA. Each fractionated population is then checked for enhanced tumorigenicity. According to this method there can be more than one tumorigenic stem cell population in the cancer cell population. Each of these tumorigenic cancer stem cell populations could then be screened to find appropriate cancer therapeutics.

According to other embodiments, a fractionated population found to comprise tumorigenic cancer stem cells can be serially fractionated to determine if there is a more specifically defined population of tumorigenic cancer stem cells. This process is necessary, because experimentally, “cancer stem cells” are defined on the basis of being capable of engendering tumor growth at low limiting dilutions of cancer cells. According to this embodiment, a cancer cell population is fractionated on the basis of differentially expressed “first” protein or RNA. If a tumorigenic cancer stem cell population is found, then the tumorigenic cancer stem cell population is again fractionated using a second protein or RNA. This second fractionated population is then again checked for tumorigenicity compared to the parent population and/or the population it was fractionated away from. If this second fractionated population comprises tumorigenic cancer stem cells, then the cancer stem cells in the cancer cell population are likely to be positive (or negative) for the two proteins used in each subsequent fractionation. This serial fractionation and phenotypic validation can be performed any number of times. In certain embodiments, the serial fractionation is performed, 2, 3, 4, 5, 6, 7, 8, 9, 10 or more times. If a single specific tumorigenic cancer stem cell population is isolated, then it can be screened against various cancer therapeutics to find a therapeutic that reduces its proliferation.

Both parallel and serial fractionation can be performed on the same cancer cell population in order to define different populations of tumorigenic populations more specifically and to find therapeutics that are effective in reducing their proliferative capacity.

Further, therapeutics can be based on the protein or RNA that the fractionation was based upon. For example, if a tumorigenic cancer stem cell population overexpresses a certain protein, then a potentially effective therapeutic would be one that reduces the expression of that protein. Likewise, if the tumorigenic cancer stem cell population underexpresses a certain protein then a potentially effective therapeutic would be one that increases the expression of that protein. For example, certain aggressive lung cancer cell subsets have lower miR34a expression than isogenic counterparts, and restoring miR34a levels to more normal levels mitigates tumorigenic potentials by aggressive cells. In certain embodiments, therapeutics that could be used to reduce the expression of a protein or RNA include antisense and RNAi based vectors. In other embodiments, therapeutics that could be used to increase the expression of a protein or RNA include vectors encoding the mRNA that expresses the protein and/or the RNA in question.

The disclosure also provides models for screening for therapeutics particularly effective for treating cancers that comprise tumorigenic cell stem cell populations positive for or highly expressing a certain protein or RNA. The models can be in vitro or in vivo models.

In vitro models include cell lines that express the same protein or RNA that a given tumorigenic cell stem cell population is positive for or highly expressing. In certain embodiments, the cell line is derived from the same type of cancer that the tumorigenic cell stem cell population is derived from. For example, for a tumorigenic cell stem cell population from lung cancer, a cell line selected from NCI-H1373, NCI-H1395, SK-LU-1, HCC2935, HCC4006, HCC827, NCI-H1581, NCI-H23, Human, NCI-H522, NCI-H1435, NCI-H1563, NCI-H1651, NCI-H1734, NCI-H1793, NCI-H1838, NCI-H1975, NCI-H2073, NCI-H2085, NCI-H2228 or NCI-H2342 (we commonly also use the following cell lines: NCI-H2122, NCI-H460; NCI-H226, NCI-H441; HCI-H647; NCI-H157; NCI-H358; NCI-H520 for various purposes) could be used wherein the cell line is transfected with the appropriate protein or RNA. Expression vectors known in the art can be used to raise the expression levels of a given protein or RNA in these cell lines. In certain embodiments, the lung cancer cell line is transfected with expression vectors encoding CD24, CD44 or Nkx2.1 (TTF-1). In certain embodiments, the cell line has reduced expression of a protein or RNA for which a tumorigenic cell stem cell population has reduced expression. In certain embodiments, a lung cancer cell line has reduced expression for miR34a. This reduced expression can be accomplished through antisense or RNAi based vectors introduced to the cell lines.

In vivo models include animals that have been genetically altered to highly express a protein or RNA highly expressed in a tumorigenic cell stem cell population or to have reduced expression of express a protein or RNA less expressed in a tumorigenic cell stem cell population. In other embodiments, the altered expression is limited to a tissue in the animal that is associated with a cancer type that the tumorigenic cell stem cell population was derived from. For example, for a tumorigenic cell stem cell population isolated from lung cancer a mouse could be genetically altered to have enhanced expression of CD24, CD44 or Nkx2.1 (TTF-1) or reduced expression of miR34a. In certain embodiments, this altered expression is in the lungs of the mouse.

Development of Cancer Biomarkers

The disclosure provides methods of developing cancer biomarkers. By isolating tumorigenic cancer stem cell populations according to the methods described above, markers on those populations can be reliably determined to be associated with those populations. Then, subpopulations that are frequently present in cancer cell populations can be detected with the biomarkers.

In certain embodiments, biomarkers are associated with tumorigenic stem cell subpopulations in certain types of cancer. For example, tumorigenic stem cell subpopulations in lung cancer include populations that more strongly express CD24, CD44, CD166, CD133, cMet, MDR1, uPAR, Sox2, or Nkx2.1 (TTF-1) or have reduced expression of miR34a. In certain embodiments, the lung cancer cell populations are isolated from malignant pleural effusions (MPEs).

Association of the presence or level of expression of certain markers can be associated with effective therapeutics, cancer stage, and disease diagnosis or disease prognosis. In certain embodiments, tumorigenic cancer stem cell subpopulations present in certain types of cancer can indicate that the cancer can be effectively treated with certain associated therapeutics. According to certain embodiments, biomarkers can be used to identify more than one subpopulation. The number of subpopulations can be 2, 3, 4, 5, 6, 7, 8, 9, 10 or more. Each of the subpopulations present may have a therapeutic that is particularly effective against the subpopulation. According to other embodiments, a single therapeutic or combination of therapeutics may be effective against a particular grouping of tumorigenic cancer stem cell subpopulations in a cancer cell population.

In certain embodiments, the presence of certain tumorigenic cancer stem cell subpopulations in a cancer cell population can be indicative of the stage of the cancer. The stage of the cancer can refer to the evolved maturity of the cancer and the likelihood that the cancer has metastasized. The stage of the cancer can be measured according to any staging system including the TNM system.

In other embodiments, the presence of certain tumorigenic cancer stem cell subpopulations in a cancer cell population can be indicative of disease diagnosis. For example, cancer cells isolated from a specific tissue or site in a subject could potentially be several types of cancer. Lung cancer cells can be selected from histo- or cyto-pathologically characterized squamous cell carcinomas, adenocarcinomas, large cell carcinomas and small cell carcinomas. It is currently not proven whether these distinct histopathological subtypes (or pathological “lineages”) have distinct cancer stem cells (or “cells of origin”). In our embodiment, we favor the association of candidate CSC-molecular signatures with specific biological behaviors as opposed to histopathological lineages or subtypes (see reference Batra and Warburton; AJRCCM, 2010 perspective). In other embodiments, the presence of certain tumorigenic cancer stem cell subpopulations in a cancer cell population can be indicative of disease prognosis. The amount of time a patient is likely to survive with the administration of different therapies may correlate with the presence of different tumorigenic cancer stem cell subpopulations in a cancer cell population.

The following examples illustrate the invention without limiting it.

### EXAMPLES

### Example 1

### Validation of CD24 as a Biomarker for Lung Cancer

C. Primary Human Lung Tumor Cells were Able to be Propagated with Orthotopic Transplantation Assay.

Primary MPE-cultures were established as previously described. The cell counts in MPEs ranged from 1.3×10⁸ to 2.5×10⁹ nucleated cells per liter, and since tumor cell counts also expanded in primary culture, tissue was not expected to be limiting. Following centrifugation (200×g, 20 minutes, room temperature), cell pellets were resuspended in a ficoll density gradient. The MPE-supematant was sterile filtered and used for the formulation of the Primary Culture Medium (PCM; DMEM-H (HyClone, UT)+30% v/v sterilely filtered MPE-fluid component+Penicillin-G/Streptomycin 1000 U/ml and Amphotericin B 0.25 mg/ml (Omega Scientific, CA)). Culture integrity and variability were monitored by microscopy. The nucleated cell pellet was extracted from the ficoll gradient, washed with DMEM-H, for initial molecular analyses and cytopathology, and several primary cultures per specimen were seeded with PCM. These were directly observed on a daily basis, and PCM was replaced at every 5-7 days. Kinetic growth analyses of primary cultures were performed on each MPE specimen. Individual MPE specimens had diverse immuno- and proliferative phenotypes. Most MPE cultures were at an ideal confluency for sorting of live cancer cell subpopulations within 5-6 weeks in culture with PCM.

MPE cultures were FACS sorted for CD24, and 50,000 CD24+ or CD24− cells were transplanted IT into NSG mice. At the 5-6 week time point, MPE cultures were split and shipped for FACS or live-sorted using flow cytometry using the FACSVantage SE system for sequencing. For FACS, labeled cells were suspended at 5×10⁶ cells/ml, filtered through 70 um nylon cell strainers, and labeled with 1 ug/ml propidium iodide to exclude non-viable cells immediately before sorting. Hematopoietic, endothelial, and immune cells were excluded from the human tumor samples using CD45, CD31, and CD11b, respectively. The sorted fractions were separated into CD24-positive and CD24-negative groups, which were separately IT transplanted into NSG mice. Mice were monitored for signs of lung tumor development and euthanized at that time. Lung tissue, local lung lymph nodes and distant organs including the liver, adrenal glands, and brain, were collected from these recipients for histology to detect primary and metastatic lesions. The ability of each cell subpopulation (CD24+, CD24−) to form metastatic lung tumors in recipients were determined by histopathological analysis performed and the transplantation results from each patient subset were compared by Fisher's exact test. Based on our murine TPC studies, CD24+ cells were expected be more efficient at human lung cancer propagation and the CD24+ population were expected to contain the cells capable of giving rise to metastases.

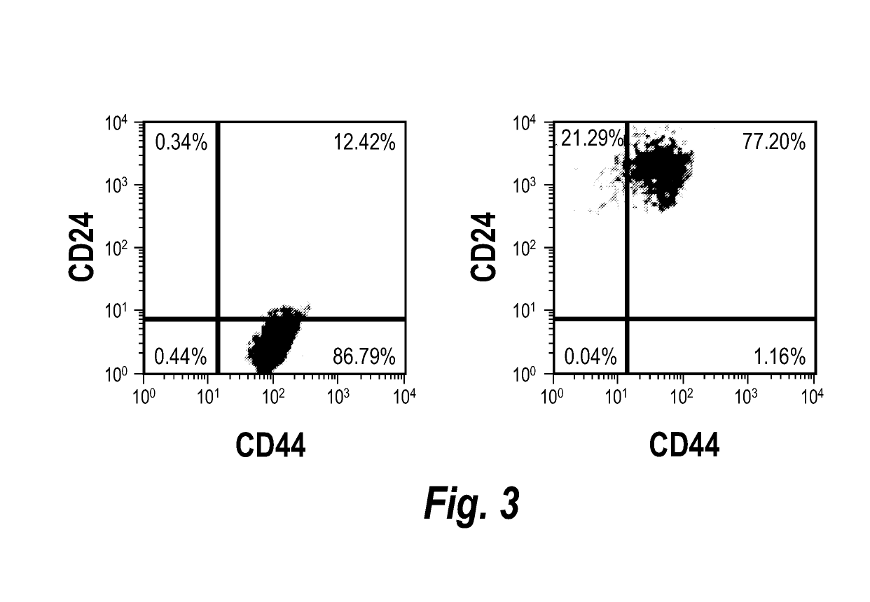

FIG. 3 shows a representative example of detection of CD24+ cells in MPE cultures from two different patients. CD24+ gating is shown for tumor cells in the live fraction. Sample 1, left, had 12% CD24+ cells and Sample 2, right, had 98% CD24+ cells. y-axis, CD24; x-axis, CD44, another candidate lung TPC marker. CD24 is a candidate marker for invasive and metastogenic lung cancer cells.

### Example 2

### Division of the Primary Cultures into Functionally Diverse Subpopulations

The goal at the outset was to develop standard operating procedures (SOPs) for the processing and culture of clinical specimens, and to determine whether tumor subpopulations that labeled for candidate CSC-phenotypes could be isolated from MPE-primary culture. IRB-approval was obtained for the informed and consented collection of MPE, 9 specimens were fully processed (see Table 3).

### A. Development of Standard Operating Procedures (SOPs)

9 clinical specimens were processed to determine whether tumor subpopulations that label for candidate CSC-phenotypes could be isolated from MPE-primary culture (see Table 3). Seven of the nine effusions were malignant on the basis of cytopathological diagnosis (Table 3). In two cases, the final cytopathology interpretation from a 50-100 ml MPE-cell button was “highly suspicious but inconclusive”; these cases were included because growth of tumor was evident in primary cultures (including in vivo in one case, data not shown).

TABLE 3
Cytopathology of MPE and their in vitro growth, AdenoCa denotes
lung adenocarcinoma, NSCLC denotes Non Small Cell Lung
Cancer (not specified), SCCa denotes lung squamous
cell cancer; ND denotes not determined.

| Subject | Cytopathology | In vitro growth |
|---|---|---|
| 106 | NSCLC | ND |
| 206 | AdenoCa | ND |
| 107 | NSCLC | yes |
| 207 | Suspicious, NSCLC | yes |
| 307 | Large Cell Ca | yes |
| 407 | AdenoCa | yes |
| 507 | NSCLC | yes |
| 607 | Suspicions, SCCa | Yes (primary passage |
| | | path c/w SCCa) |
| 707 | Poorly differentiated SCCa. | yes |

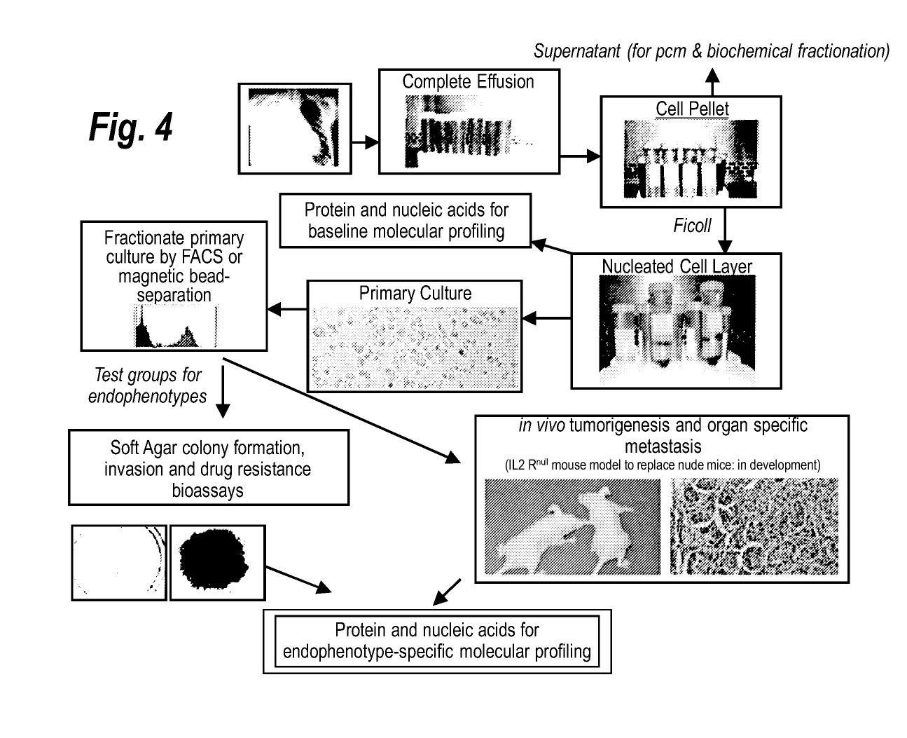

MPE were extracted from the subject and separated from a diagnostic-aliquot that was sent to the pathology service. The research aliquot was processed and cultured as summarized in FIG. 4. 3 different MPEs in primary cultures containing either 100%, 70%, 50%, 30% and 10% MPE-fluid component were monitored. Culture integrity and variability were evaluated by microscopy. The fractions of floating dead cells (by trypan blue staining) were measured in each condition. There were no qualitative differences in culture integrity or variability, and no quantitative differences in floating dead cells amongst the 70%, 50%, and 30% v/v MPE-fluid conditions. The 100% and 10% v/v MPE-conditions had an increase in cell death in 2/3 effusions. Thus, 30% v/v MPE was selected.

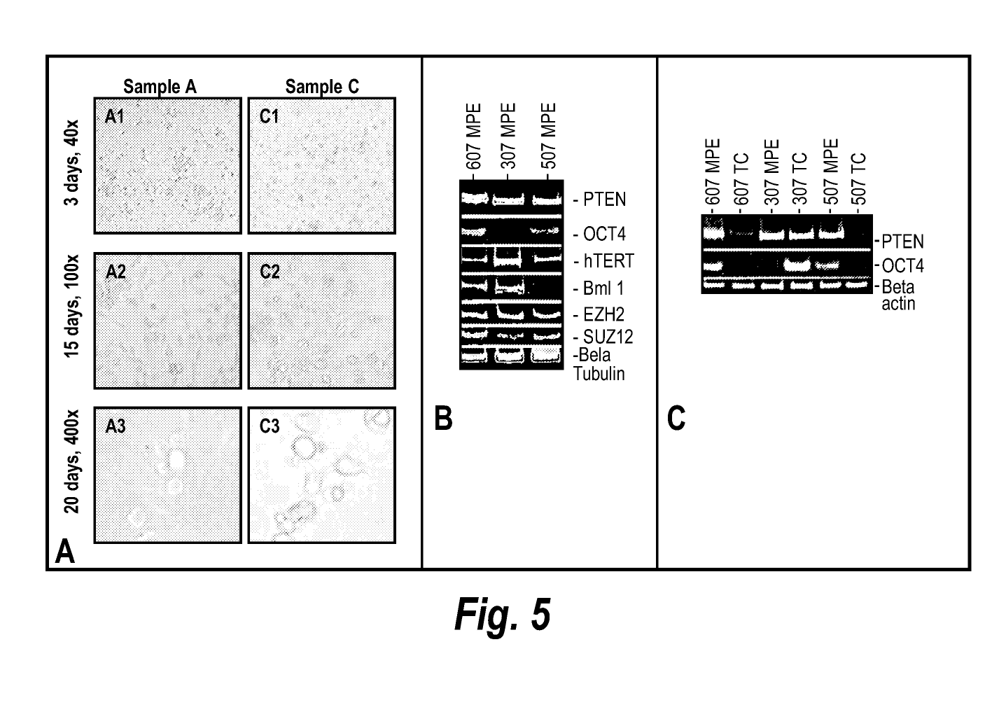

The nucleated cell pellet (counts ranged from 1.3×10⁸ to 2.5×10⁹ cells per liter of effusion) was extracted from the ficoll gradient, washed with DMEM-H, and aliquots were separated for storage, cytopathology, DNA/RNA/Protein extraction, and primary cultures. Culture conditions can be varied to study the impact of the tumor microenvironment (TME) on the candidate CSC-phenotype. Cell button pathology suggested that the MPE tumor was variably contained within indistinct clusters of cells of varying compositions, or as well organized spheroids. MPE-primary cultures always displayed diverse colony-morphologies (exemplified by FIG. 5A). The primary cultures typically displayed slow growth rates and evolved over time, taking several weeks to “mature” (reach ˜60-70% confluence in a T225 culture vessel). Over this interval, the clusters and spheroidal structures that were observed in the initial MPE-cytopatho/ogy were not well conserved.

### B. Fractionating MPE-Tumors.

A novel way to culture primary MPE-tumor specimens was developed in a manner that maintains intratumoral heterogeneity, including cells bearing candidate CSC-markers, over time. Primary cultures were generated in vitro from MPE-specimens with high efficiency (7/7 attempts), using an autologous tumor microenvironment or TME (note that in vitro cultures were not attempted with the first two MPE-specimens). The Autologous TME (primary culture medium) was comprised of the MPE fluid and the non-epithelial nucleated cell population that was extracted with the tumor.

The MPE-primary cultures grown in pcm (DMEM-H supplemented with 30% MPE-fluid component and antibiotics) displayed a phenotypic heterogeneity that had not previously been examined (FIGS. 5A, 5B). Such cultures were always comprised of colonies with varying morphologies (floating aggregates that excluded trypan blue, giant cell colonies, fibroblastoid and cobblestoned clusters) all within the same flask. The colonies appeared to expand at varying rates despite being in a “common” environment.

To test if these biomarkers of candidate CSC could be detected in MPE-tumors, RNA extracted from the nucleated cell fractions was used for RT-PCR. These candidate CSC-biomarkers were evidenced in MPE-tumors (FIG. 5C). Thus, contrary to conventional postulates regarding the CSC niche environment, the candidate CSC-phenotype was preserved in MPE cultures despite the inflammatory milieu of these specimens. These results also established the feasibility for dynamically tracking these biomarkers as experimental changes into the pcm-culture conditions were introduced in efforts to enrich for the CSC-phenotype. Therefore, molecular signatures of CSC were evident in MPE-tumors.

There was a dynamic interplay between the TME and the CSC-phenotype in culture. Expression of CSC biomarkers was observed to dynamically change as MPE-tumors were longitudinally monitored in primary culture (FIG. 5C). For example (FIG. 5C), the PTEN marker diminished in two of the three specimens, and Oct 4 expression increased in primary culture 307 with dynamic monitoring.

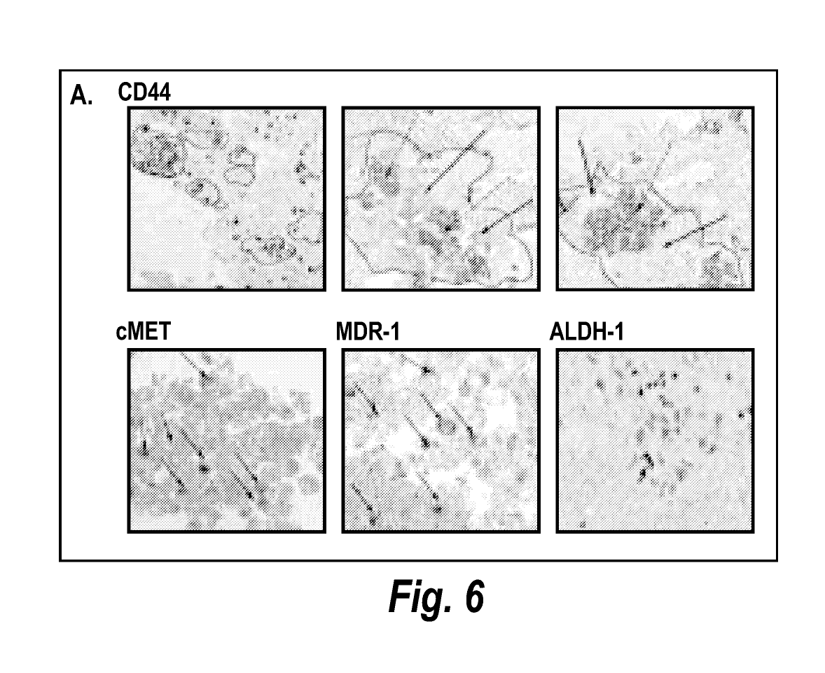

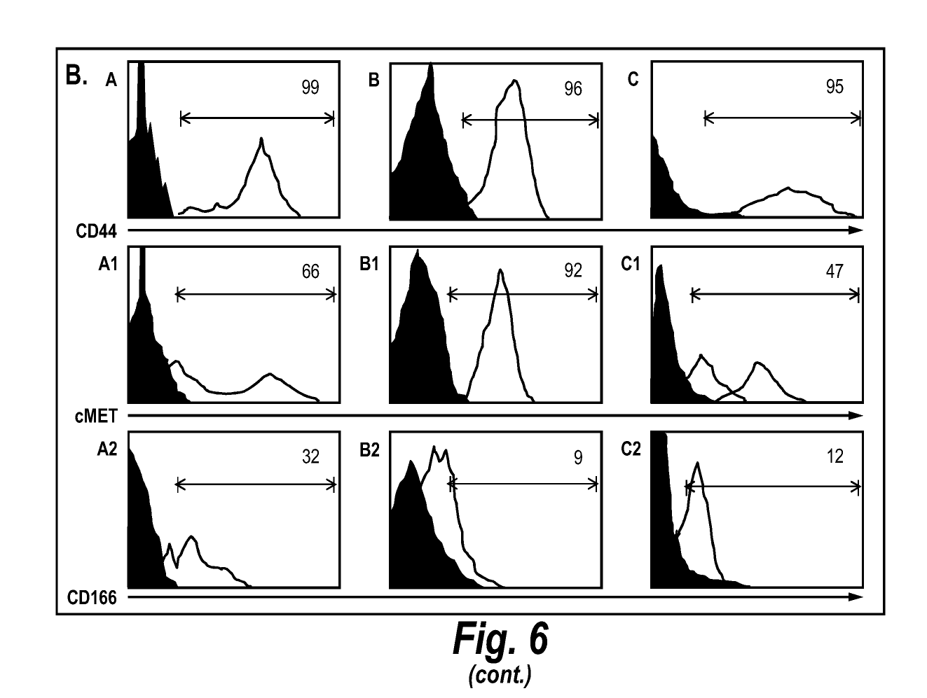

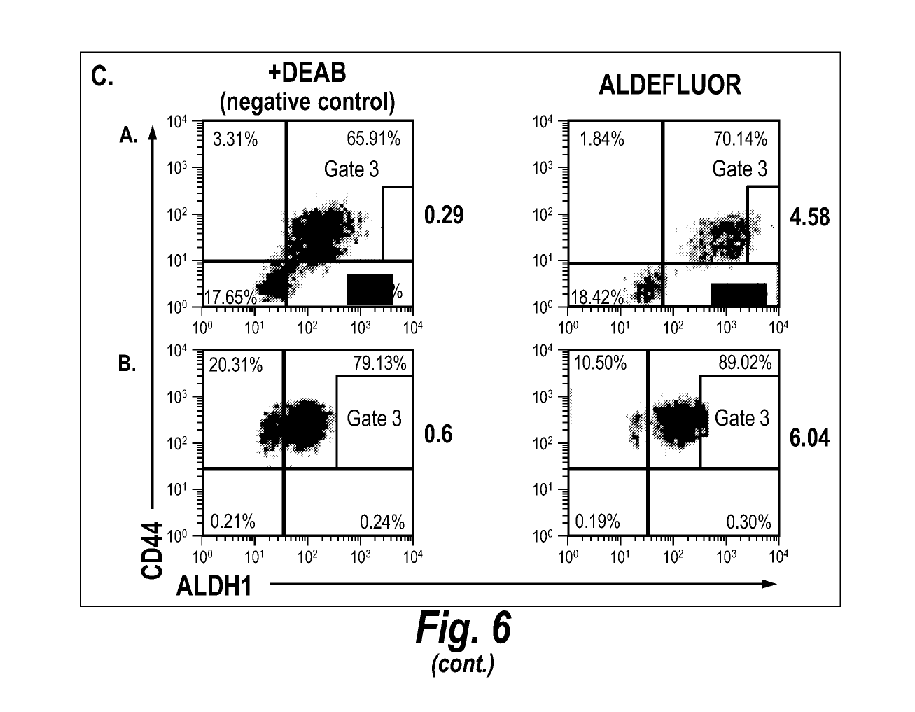

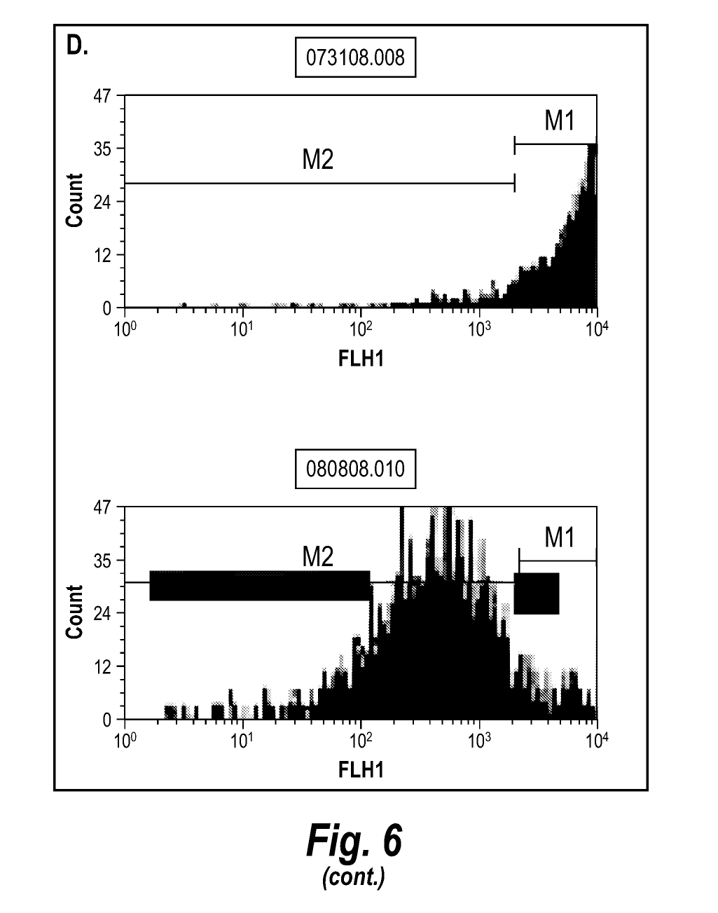

Because CD44 is a useful CSC-marker, the MPE-specimens were examined for its fractional expression. All MPE specimens examined displayed a CD44+ fraction, ranging from an estimated 8% to 45% of nucleated cells by immunohistochemistry (IHC) (FIG. 6A). In addition, cell fractions also displayed other candidate CSC-markers cMET and MDR-1 (FIG. 6A).

Differences in xenobiotic metabolism of cells were also used to segregate CSC from the tumor mix. The Aldefluor™ assay (StemCell Technologies) was used to segregate candidate CSC on the basis of ALDH1A1 activity. Similarly, cells that immunolabel for ALDH1 were observed in MPE-tumors (FIG. 6A). Thus, CSC-surface and metabolic biomarkers were evident in MPE-tumor.

### C. MPE-TME Characteristics.

The MPE-tumor microenvironment (TME) was observed having discrete cellular [tumor and stromal-cell (leukocyte/mesothelial/fibroblastic)] and non-cellular fractions. The total cell counts in the MPE ranged from 1.3×10⁸ to 2.5×10⁹ nucleated cells per liter of effusion, with the largest majority comprised of the tumor cell population in most cases. However, there were also significant contributions from resident and circulating leukocytes, and stromal cells in the effusions (Table 4).

TABLE 4
Non epithelial Cellular Composition of the MPE. Counts and differential
of Giemsa-Wright stained cytology slides were obtained in the
VAGLAHS hematopathology laboratory. The numbers in column two
indicate the numbers or percentages of various cell types. Column 3
is the fraction of MPE in which the various cell types were identified.
These counts are typical of MPE.

| MPE Cell Counts | Mean ± Standard | MPE in which |
|---|---|---|
| and Differentials | Deviation (n = 9) | wbc-diff is ≧1% total |
| RBC | 158061 ± 172443 rbc/l | N/A |
| WBC | 681 ± 560 wbc/l | 9/9 |
| Lymphocytes | 60 ± 26% | 9/9 |
| PMN | 23 ± 27% | 8/9 |
| Monocytes | 3 ± 3% | 9/9 |
| Macrophages | 6 ± 8% | 8/9 |
| Mesothelial cells | 4 ± 4% | 8/9 |
| Other (eosinophils, | 6 ± 13% | 4/9 |
| plasma cells) | | |

The MPE-TME was surveyed with a commercially available multiplex panel (3 different undiluted MPE samples were run in duplicate using the LINCO™ 29-plex kit, and analyzed on a Luminex® 200™ platform). Numerous candidate growth factors, cytokines, and chemokines were found in the MPE-fluid milieu (Table 5) that might modulate the CSC phenotype. These factors were already implicated in the pathogenesis of effusions and/or migration of tumor cells into the pleural space, and/or tumor progression. For example, VEGF, PGE2; IL-6, TNFα, and/or SDFla were implicated in the induction of vascular permeability and/or the recruitment of tumor cells into the pleural space. Notably, several cytokines displayed very high concentrations [≧10 ng/ml (Table 5, in red)].

TABLE 5
MPE Cvtokine/chemokine concentrations as compared to a serum
matrix background and a 10 ng/ml standard (*denotes a relatively
poor standardization of the IL-1ra IL-10 and sCD40L
measurements in this preliminary analysis).

| Cytokine/ | Serum matrix | MPE | Standard |
|---|---|---|---|
| chemokine | background | (Mean ± Standard Deviation) | 10,000 pg/ml |
| IL-13 | 28 | 105 ± 175 | 92 |
| II-1 | 42 | 1886 ± 3393 | 274 |
| IL-1ra | 41 | 948 ± 1646 | 64* |
| IL-10 | 10 | 212 ± 87 | 11* |
| IL-5 | 18 | 3402 ± 5783 | 3428 |
| IL-6 | 25 | 21116 ± 3290 | 884 |
| IL-8 | 24 | 2709 ± 1107 | 2321 |
| IP-10 | 44 | 16205 ± 7404 | 1006 |
| MCP-1 | 18 | 12653 ± 5586 | 2698 |
| sCD40L | 44 | 196 ± 146 | 57* |
| VEGF | 39 | 3269 ± 4482 | 140 |

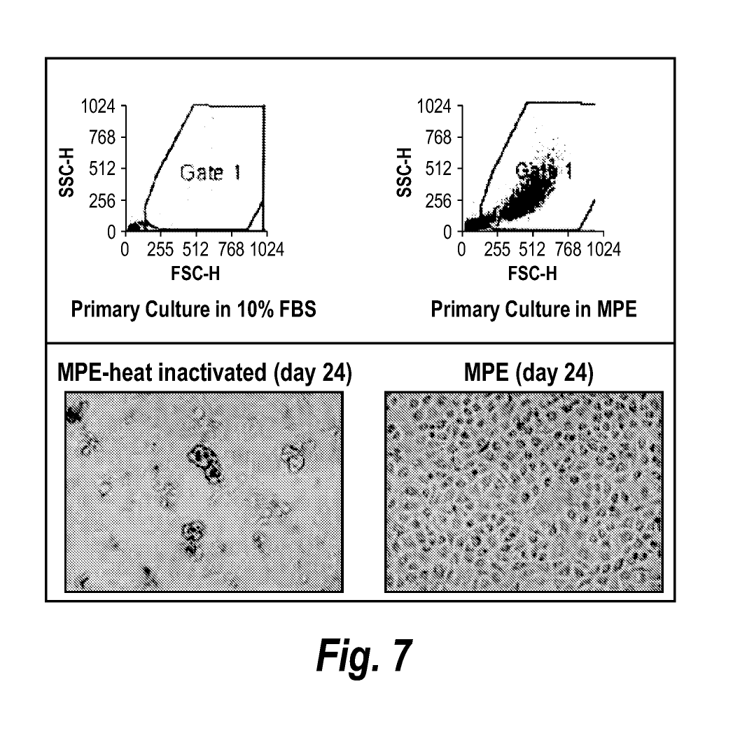

The MPE fluid clearly contributed to the robustness and heterogeneity of primary cultures (FIG. 7). Primary cultures established in pcm (with 30% v/v MPE-fluid) displayed far greater heterogeneity and robustness than parallel cultures established in fetal bovine serum (10% v/v) (FIG. 7A). Cell viability was also strongly affected by heat-inactivation of factors in the MPE (FIG. 7B).

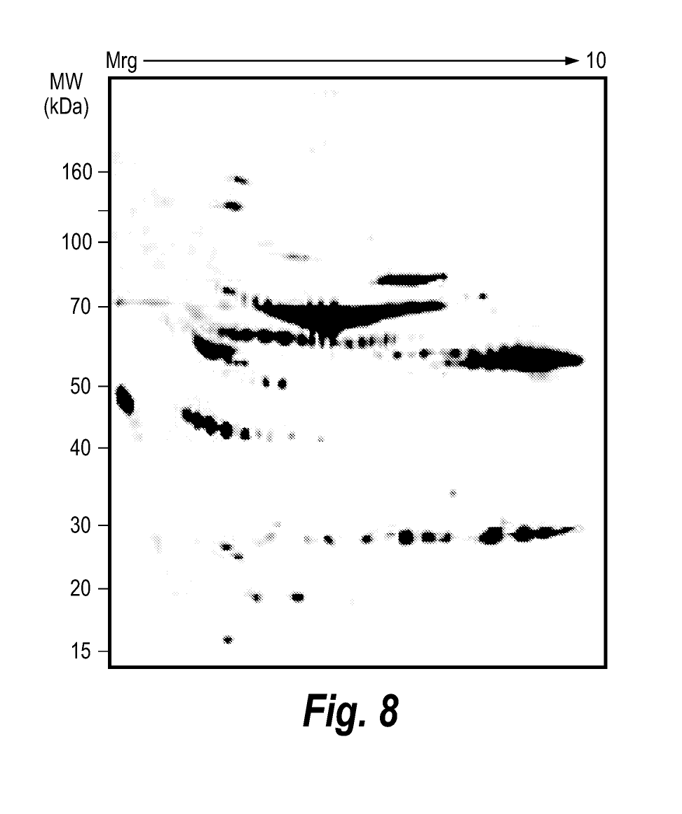

The MPE-fluid component was examined by 2-D PAGE analysis (FIG. 8). The most abundant species in the MPE were confirmed to be plasma derived components (FIG. 8).

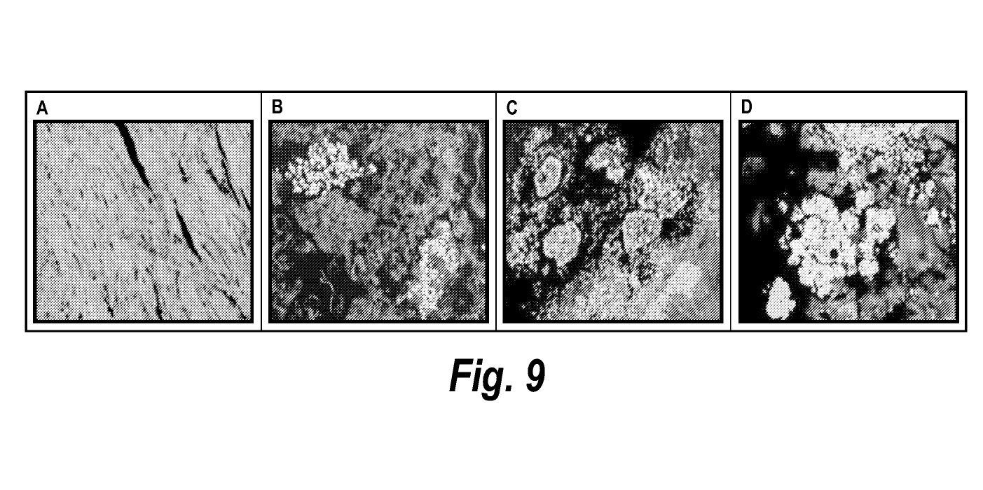

At the light microscopic resolution, using the Masson's trichrome stain in Bouin's-fixed cytopathologythe, tumor cell clusters in MPE did not stain positively for fibrillar collagen (FIG. 9).

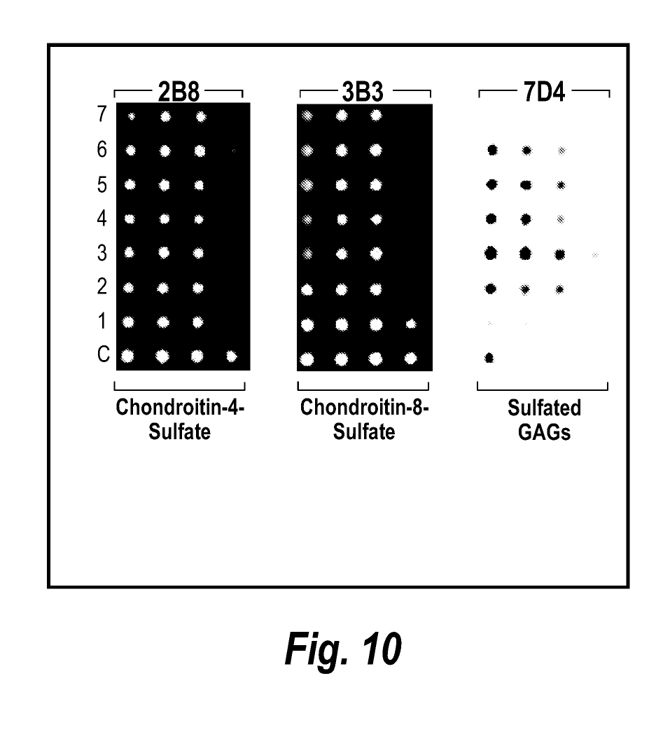

Soluble CS-PGs were found to be a prominent component of MPEs (FIG. 10), and were readily detected, even at serial dilution of the effusions to 1:50,000. Gel filtration (Sepharose CL-6B) analysis of MPE indicated that the CS moieties started eluting with the void volume, and continued to elute until the salt peak was reached, suggesting that CS-PGs in MPE were a heterogeneous group, represented by elements with molecular weights of over 1 million to smaller fragments of less than 10,000 daltons. When hexuronic acid and total GAG-sulfation content were used to biochemically quantify GAG-concentrations in MPE, it was observed that the hexuronic acid concentrations in the 01 and 02 fractions of CsCI density gradient purified effusions ranged from 6.6 to 58.4 pg/ml (mean±SEM=27.37+8.69 μg/ml), and that the sulfated GAG concentration in the effusions was determined to be 30+7.9 μg/ml. Assuming that 40% of the isolated PG/GAGs is hexuronic acid, the mean PG/GAGs concentration in the soluble component of MPE was estimated to be 68 μg/ml.

In summary, the MPE was found to be comprised of heterogeneous subpopulations of tumor cells. Candidate CSC cells can be found in clinical samples, and can be maintained in MPE-primary cultures. Using cell surface and metabolic markers that distinguish CSC, this subpopulation can be live sorted from the vast majority of tumor cells in the MPE-mix.

### Example 3

### Validation of CD44 as a Biomarker for Lung Cancer

### A. Poor Engraftment Efficiency of Unselected MPE Tumors

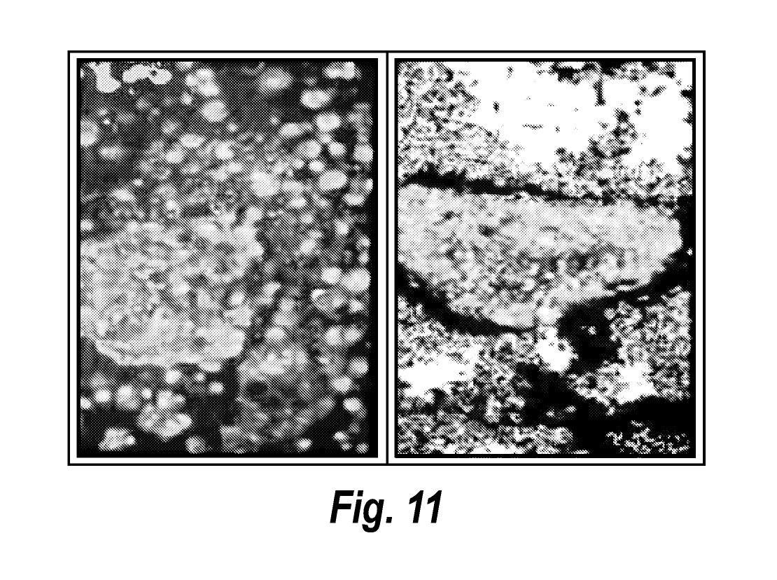

MPEs were collected from subjects who were veterans and active or former smokers. 9 different MPEs were processed, wherein 8 MPEs were confirmed by diagnostic cytopathology, 1 specimen was confirmed by in vitro growth that formed tumors on in vivo transplantation) (see Table 6). An additional 4 effusions were processed but are not included in analysis (cytopathology and primary culture were negative, or the effusion was transudative and paucicellulular with less than 1×10⁶ cells per liter). The cell counts in the MPE ranged from approximately 1.3×10⁸ to 2.5×10⁹ nucleated cells per liter of effusion, and the MPE-tumor clusters were comprised of cells which are replication competent, not senescent (FIG. 11).

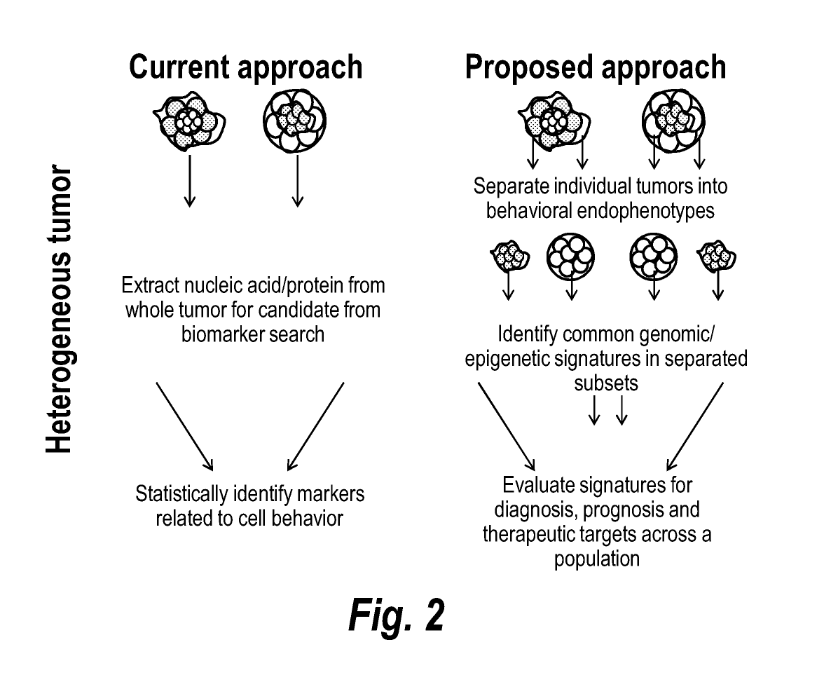

TABLE 6
Depicts the clinical characteristics of the patient and the specimen that was used to
generate primary in vitro and in vivo cultures. (*denotes that although primary
engraftment efficiency was nil, tumors formed in nude mice from cells derived
from an in vitro culture. ND denotes not determined, AdenoCa denotes
lung adenocarcinoma, SCCa denotes lung squamous cell cancer).

| | | Estimated # of cells | Primary engraftment | In vitro |
|---|---|---|---|---|
| Subject | Dx/Cytopathology | injected | efficiency | growth |
| 106 | T4N2M1 NSCLC, cytopath+ | 1 × 10⁵ | 0/1 | ND |
| 206 | Cytopath + AdenoCa | 5 × 10⁵ | 0/2 | ND |
| 107 | NSCLC, cytopath + (see FIG. 3) | 5 × 10⁶ | 2/2 | yes |
| 207 | Stage 4 NSCLC, cytopath inconclusive | 5 × 10⁶ | 0/2* | yes |
| 307 | Large Cell Ca cytopath + poorly | 1 × 10⁷ | 2/2 | yes |
| | differentiated Ca. (see FIG. 2) | | | |
| 407 | Cytopath + AdenCa (see FIG. 2) | 1 × 10⁷ | 1/1 (transient) | yes |
| 507 | Cytopath + NSCLC | 5 × 10⁶ | 1/1 | yes |
| 607 | Stage 4 SCCa, cytopath inconclusive | 5 × 10⁶ | 1/1 (transient) | Yes (primary passage |
| | | | | path c/w SCCa) |
| 707 | Treatment refractory NSCLC, cytopath | 5 × 10⁶ | Primary monitoring | yes |
| | poorly differentiated SCCa. | | in progress | |

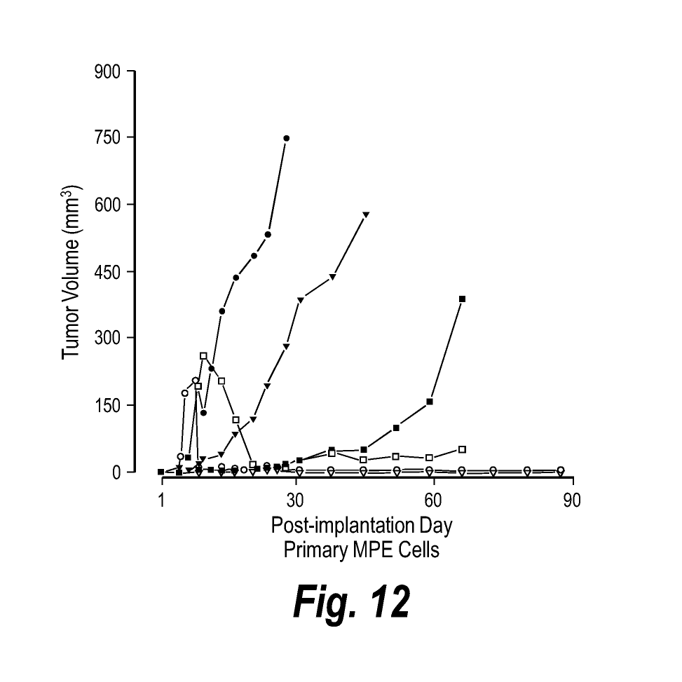

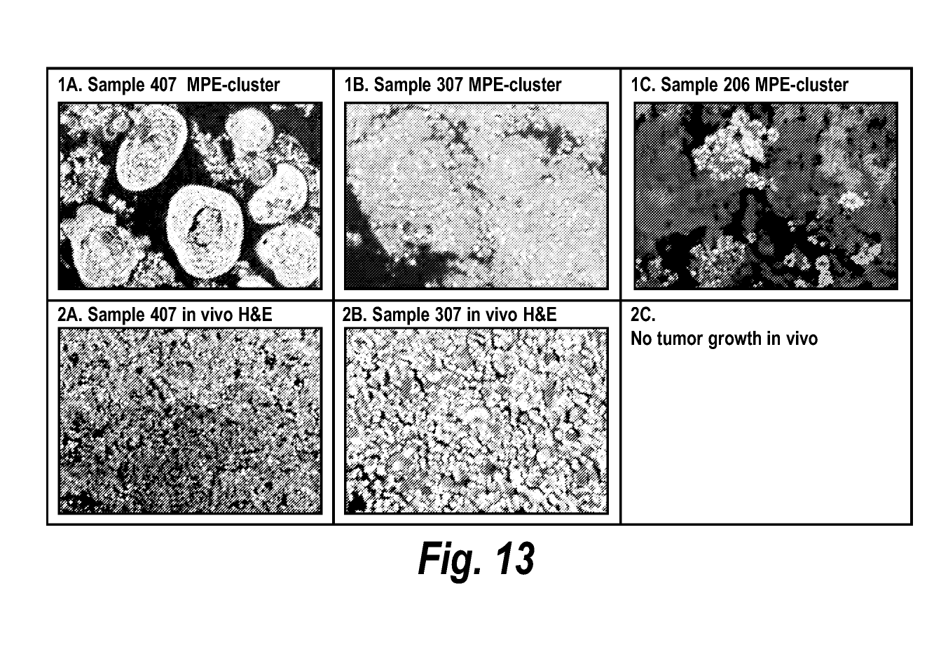

Cells were grown in autologous medium (DMEM-H/MPE in a 2:1 Ratio) to establish cultures in vitro in every case. Unselected cells between 1×10⁵ and 1.1×10⁷ were injected (initial doses were based on engraftment efficiencies for MPE-derived breast Ca cells, and were increased due to the observed failure of engraftment) into the post flank region of nu/nu mice. Tumors were generated in 3 out of 9 cases (see Table 6 and FIGS. 12 and 13). Three of the MPE did not yield tumors (over seven months of monitoring), and 1 MPE implantation is still being monitored for primary tumor growth. In another two cases, the tumors grew rapidly, but then regressed to imperceptibly small nodules (in 1 case, the residual tumor appeared avascular). Importantly, three of the MPE cell transplantations resulted in progressive tumors which grew to beyond the ˜500 mm³ stage in subcutaneous xenografts, and which were extirpated and processed for pathology and secondary tissue digestion for serial transplantation. Thus, a subpopulation of cells (putative LCIC) in the total cellular mix of some MPE, when injected into mice with their niche intact, was proved being capable of generating SQ-tumors in nude mice.

### B. Efficient Engraftment Efficiency of Selected MPE Tumors

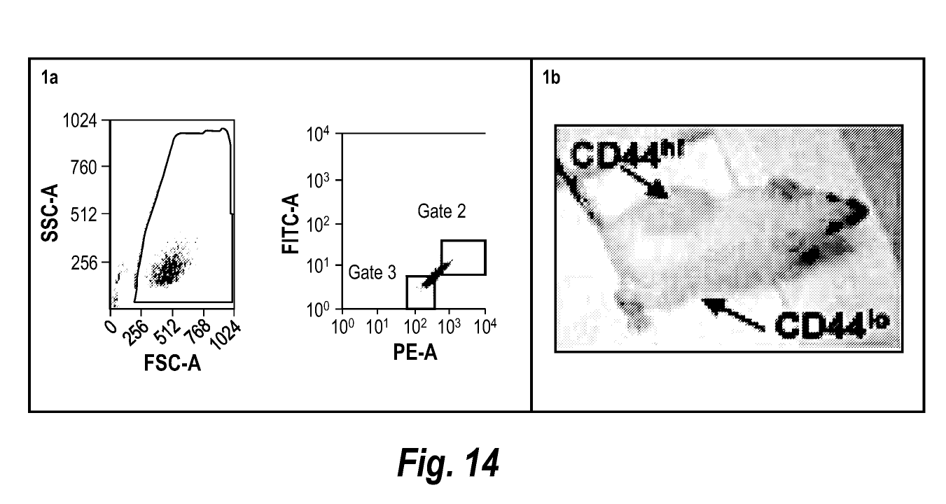

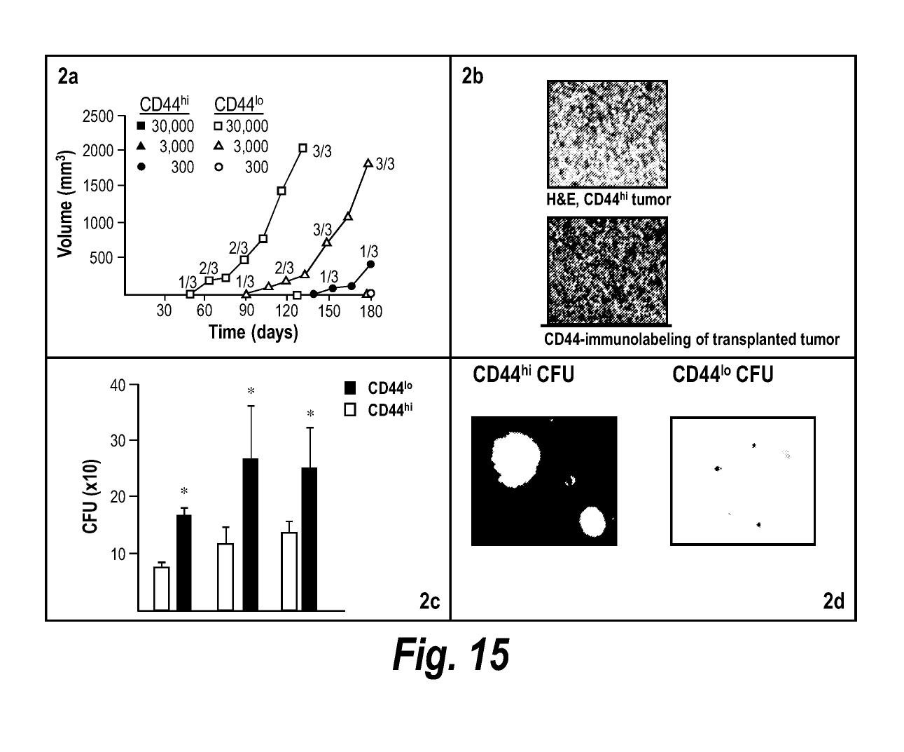

Isogenic tumor cells separated on the basis of surface markers (e.g.: CD44) were compared to determine if they display differences in tumorigenic potential (FIG. 14a, 14b). When MPE tumor was separated on the basis of CD44-expression (FIG. 14a), 30,000 CD44^{hi} cells (14a; gate 2) readily formed a tumor in the left flank, whereas 30,000 isogenic CD44^{lo} cells (14a; gate 3) were unable to form a tumor in the right flank of a SCID mouse (FIG. 14b). Thus, the CD44^{hi} MPE-tumor cell population was more tumorigenic than an isogenic CD44^{lo} cell population. Similarly, 3000 CD44^{hi} cells were capable of tumorigenesis in this setting, although the time frame to the development of tumor is more prolonged.

NOD/SCID IL2γR^{null} mice were used for tumor cell implantation. Tumors were formed in vivo with 300 selected CD44^{hi} cells in about 4-6 months, but not with 30,000 CD44^{lo} cells from the same MPE-primary culture (FIG. 15a). The tumors that formed from CD44^{hi} cells displayed both morphological and antigenic (for CD44 and ALDH-expression) variability (CD44 shown in FIG. 15b). In addition, CD44^{hi} cells not only formed more anchorage independent (soft agar) colonies (FIG. 15c), but those colonies appeared to display significant differences in average size and optical densities (FIG. 15d) as well, as compared to CD44^{lo} cells. Thus, the CD44^{hi} subsets in individual lung tumor populations were proved to have increased tumorigenic potential, and, given the result depicted in FIG. 15a, the CD44^{hi} subset in individual tumors very likely contained tCSC.

To determine the extent to which surface CD44^{hi} and Kras mutations/Kras activity co-associate to drive the “tumorigenic potentials” by lung cancer cells, the association of Kras mutations with the CD44^{hi} phenotype was tested in lung adenocarcinomas. The distribution of Kras mutations was found similar in the CD44^{hi} and CD44^{lo} subsets (Table 7).

TABLE 7
Distribution of Kras mutations in the CD44^{hi} and CD44^{lo} subsets.

| Sample | Cell Population | KRAS mutation analysis |
|---|---|---|
| 1 | CD44^{hi} | Wild-type |
| | CD44^{lo} | Wild-type |
| 2 | CD44^{hi} | Wild-type |
| | CD44^{lo} | Wild-type |
| 3 | CD44^{hi} | Mutated (Gly 12→Cys) |
| | CD44^{lo} | Mutated (Gly 12→Cys) |
| 4 | CD44^{hi} | Mutated (Gly 12→Ser) |
| | CD44^{lo} | Mutated (Gly 12→Ser) |
| Normal fibroblast | CD44^{hi} | Wild-type |
| | CD44^{lo} | Wild-type |

C. Dynamic Migration of Surface Phenotype and mRNA-Expression of MPE Tumor Cells in Culture

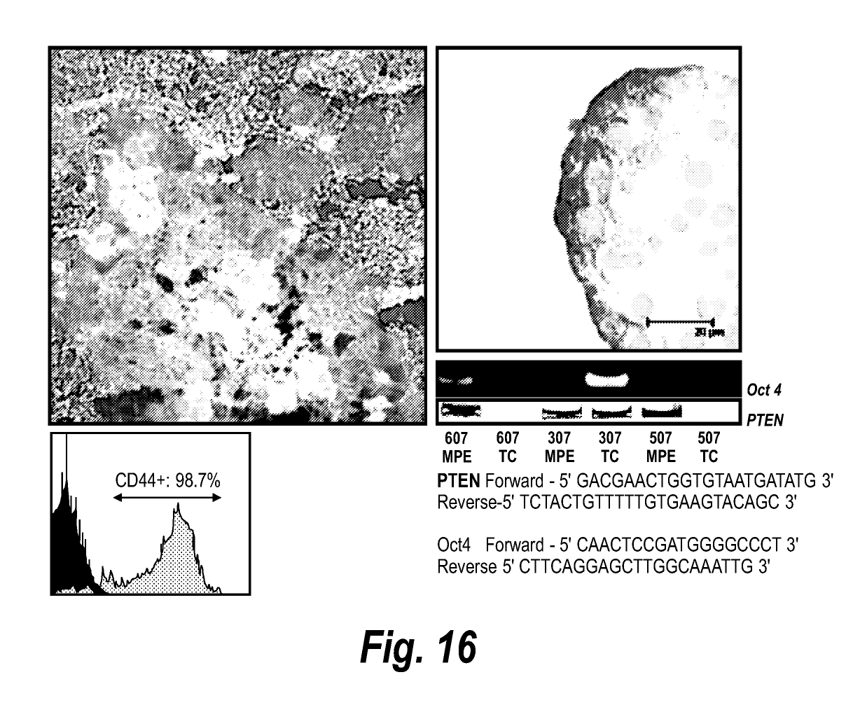

CD44-expression (subject 107, IHC depicted in FIG. 16a) was found in approximately 26% in a sample of the initial MPE-tumor cell cluster isolate. The same tumor, when grown in vitro in medium containing autologous MPE-supernatant, exhibited 98.7% CD44 labeling (by FACS) 5 days later (FIG. 16b). Thus, there was likely a drift in CD44 expression, and the cultured cells likely had undergone a dynamic change while in primary culture.

The primary-cultured CD44+ population was observed also being concomitantly cMET+ (61%), CD166+ (48%), MDR1+ (44%), uPAR+ (46%), and CAR+ (84%). Two months later, after 6 serial passages, the cells still remained CD44+ (94%), 48% cMET+, and 32% uPAR+. Dynamic fluctuations in cell surface phenotype were also observed when cells were passaged through the mouse. With specimen 307, whereas CD44 expression was not significantly different between primary cultured cells in vitro and the mouse passaged culture in vivo (96% and 98% respectively), the expression of MDR1 and uPAR were markedly different between the in vitro (9.4% and 8.5% respectively) and in vivo settings (50% and 48% respectively).

There seemed to be distinct zones of PG/GAG distribution in the pericellular matrix. The labeling of MPE-tumor clusters with the murine monoclonal 4C3 (IgM, κ-isotype) in MPE-tumor clusters (sample 407) distinguished regions of varying CS-sulfation motifs in matrix PG (FIG. 16e). In this setting, 4C3 recognized distinct and “native” CS sulfation motifs, which was likely associated with perlecan and/or versican, or CS-substituted decorin and/or cell surface serglycin.

PTEN and Oct 4 signals were clearly detected from RNA preserved from MPE-tumor clusters isolated from patients, and from the primary cultures that were derived thereafter (FIG. 16d). In two out of the three MPE specimens (samples 607 and 507) depicted here, PTEN and Oct 4 expression seemed to diminish as primary cultures were established. However, it appeared that in sample 307, Oct 4 expression emerged in primary culture (whereas PTEN expression remained similar to that in the primary isolate; FIG. 16d).

These experiments suggested that the selection and characterization of putative LCIC was dependent of markers and expressional programs which might vary dependent on the context in which the tumor cells were being fractioned and/or cultured. More importantly, the association of these markers with the clonogenic/tumorigenic phenotype might need to be empirically established in a dynamic manner.

### Example 4

### Validation of Nkx2.1 as a Biomarker for Lung Cancer

For lung cancer, Nkx2.1, SOX2, and Grhl2 are candidate markers for study. Nkx2.1 is a developmentally expressed TF important for lung development, growth, and repair, and it is also highly expressed in lung adenocarcinoma (adenoCa). 10-15% of lung adenoCa have Nkx2.1 gene amplification, and 80% of lung adenoCa are characterized by immunolabeled Nkx2.1+ cell fractions. SOX2, marks early progenitor cells in the oropharyngeal and tracheal epithelium. Amplification of the SOX2 gene is frequently associated with lung squamous and small cell cancers, and SOX2 also induces a pluripotency state in differentiated somatic cells. Grhl2 is an essential TF that controls epithelial morphogenesis and differentiation, and it cooperates with Nkx2.1 to control cell adhesion, plasticity, and motility. However, the roles of these developmental TFs for directing subtype-differentiation or behavioral plasticity in lung cancers are not clear. Using a novel “transcriptional sorting” paradigm, we seek to define these roles.

To implement the strategy, lentiviral vectors have been developed that encode fluorescent reporter genes downstream of Nkx2.1 response elements from the Surfactant Protein C gene. Protocols have already been developed to efficiently transduce primary lung cancer cultures with like vectors.

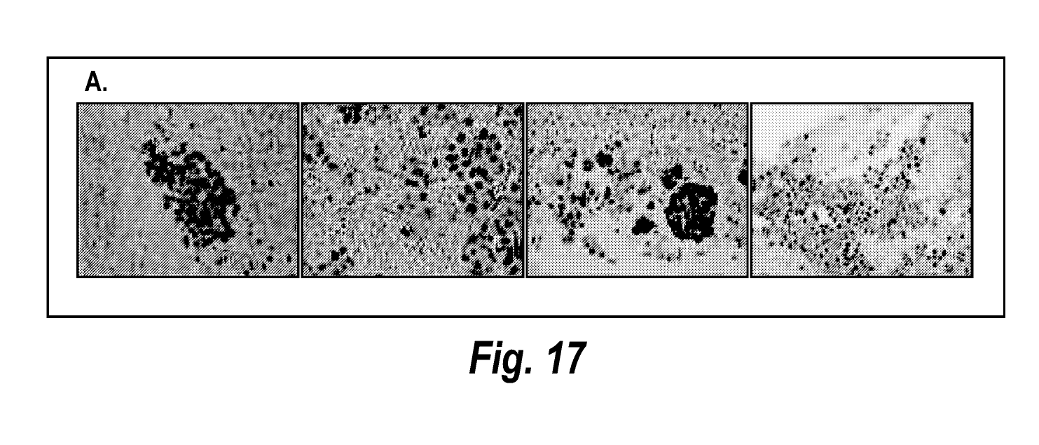

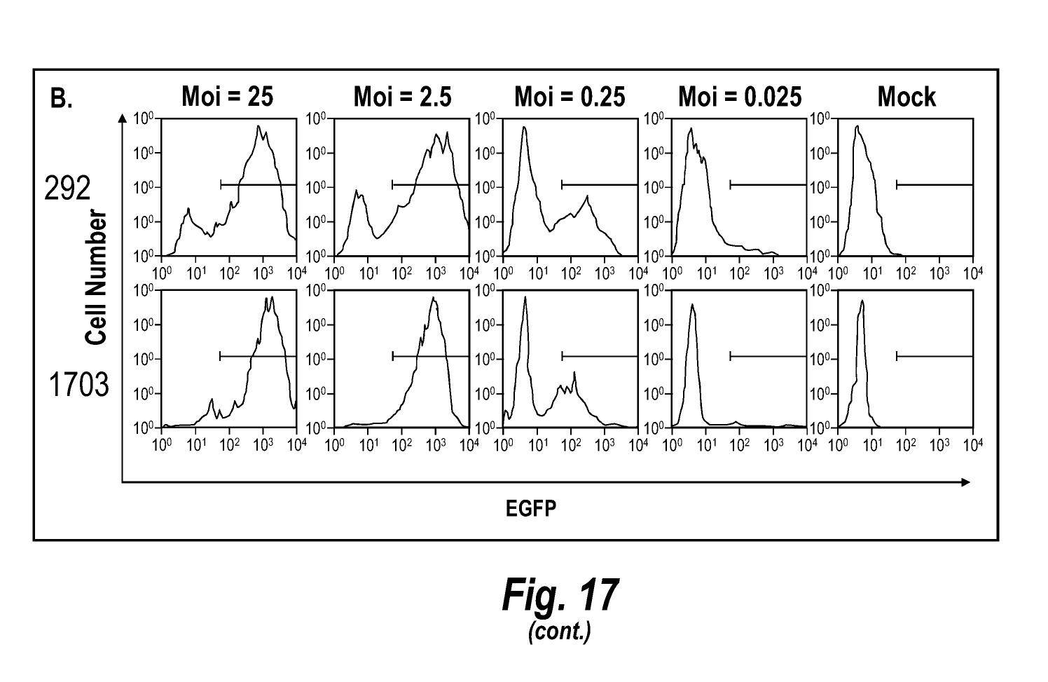

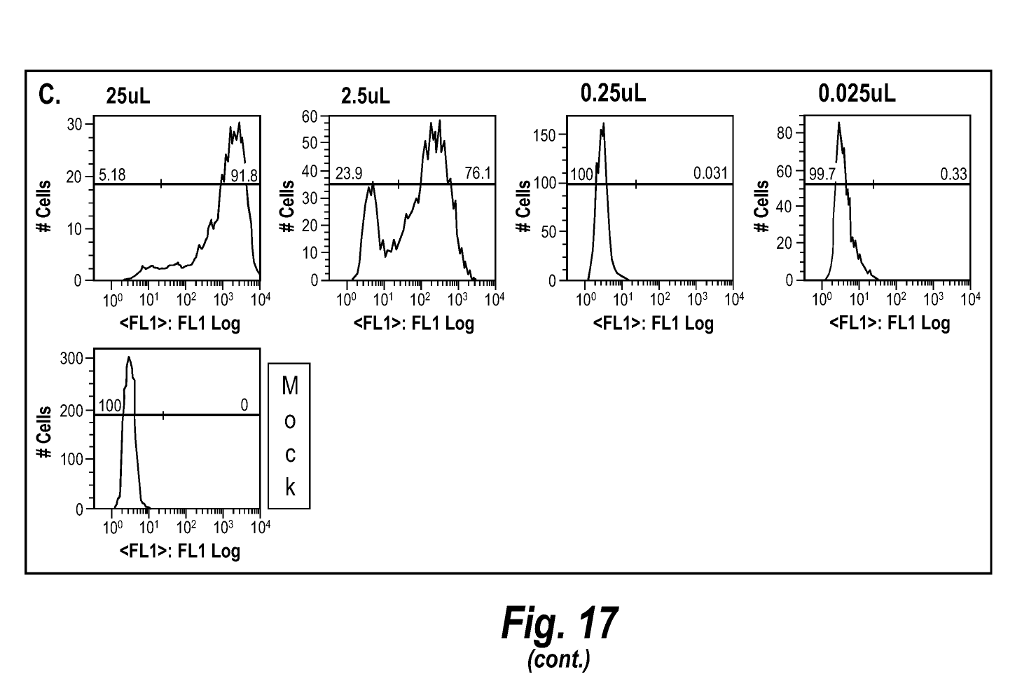

Fractional immunoreactivity for Nkx2.1 (TTF-1) was observed in malignant pleural effusion (MPE) specimens (FIG. 17A). This immunoreactivity was limited to tumor-cell subpopulations, because immunohistochemistry for lymphocytic (CD3), histiocytic (CD68), and mesothelial (calretinin) labels in serial sections was not associated with the cell populations that labeled for Nkx2.1.

The FUGW lentiviral vector (LV) prototype that was used to target Nkx2.1-expressing subpopulations was found efficiently transduce model lung cancer cells (FIG. 17B), and MPE-primary cultures (FIG. 17C).

### Example 5

### Validation of miR34a as a Biomarker for Lung Cancer

Reduction of Tumorigenesis of CD44^{hi} Cells when Transfected with miR34A.

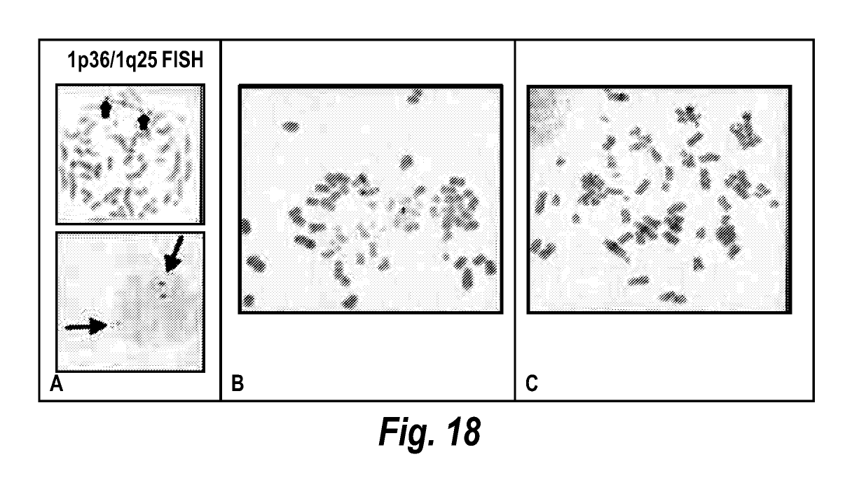

The CD44^{hi} and CD44^{lo} subsets in MPE-primary cultures were observed differ with respect to whole genome methylation profiles, and in the expression of specific miRNAs. For example, the CD44^{hi} subset displayed decreased miR34a expression. Screening FISH (fluorescence in situ hybridization) analyses were performed. Intriguingly, using probes for chromosome 1, it was observed that there were deletions and LOH in the MPE tumor samples when using probes for 1p36 region (as compared to a 1q25 region probe as a control; FIG. 18).

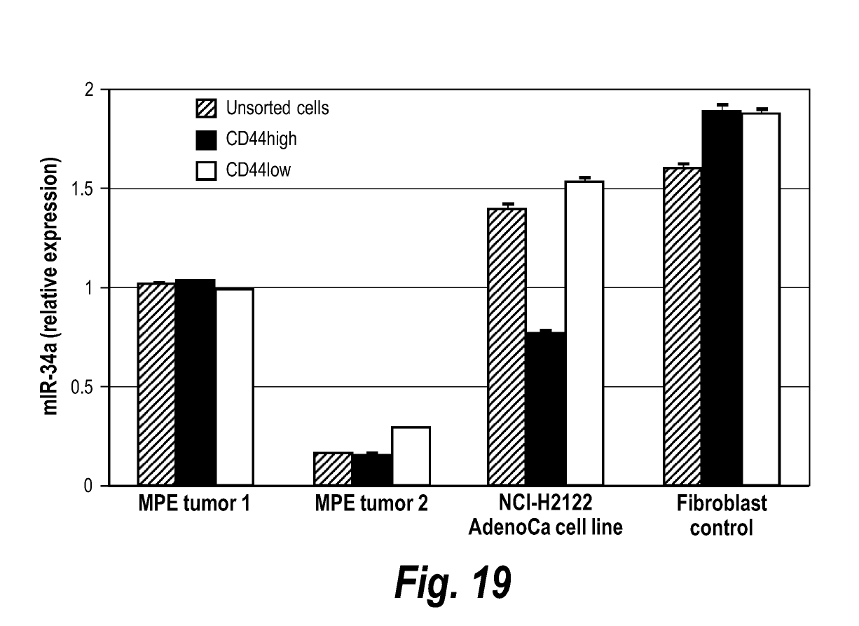

Differences in miR34a expression in the CD44^{hi} versus CD44^{lo} subsets of lung cancers were tested. Indexed to the small nucleolar RNA-RNU48, quantitative RT-PCR analysis consistently showed reduced expression of miRNA34a (as compared to normal fibroblasts) in lung cancer cells (both in primary MPE cultures as well as a representative cell line) (FIG. 19). Importantly, intra tumoral differences in miR34a were also evident (albeit variably) when the aggressive (CD44^{hi}) subsets were compared with the CD44^{lo} subset (FIG. 19; see MPE tumor 2 and H2122 cell line). Thus, miRNA34a plays

a role in tumor suppression in lung cancers, and dysregulated miR34a expression was not likely the only molecular abnormality that was associated with the aggressive (enhanced colony-forming) CD44^{hi} phenotype.

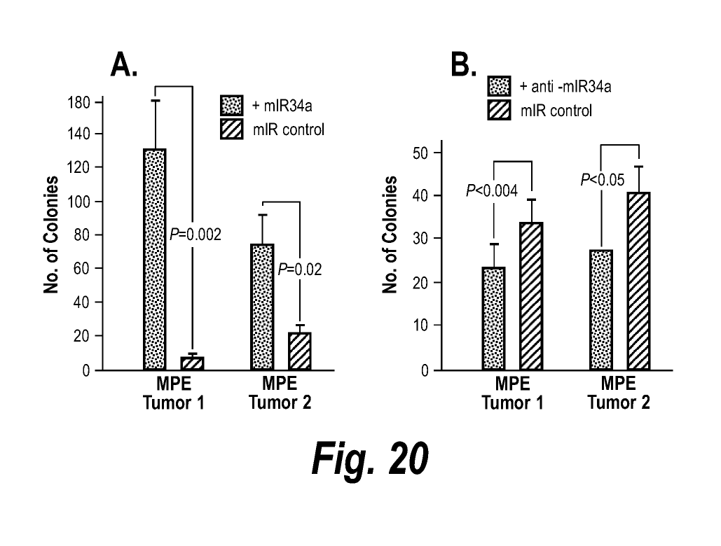

When CD44^{hi} cells were transfected (lipofectamine) with miR34a, they displayed an 80-95% reduction in soft-agar colony formation in replicate wells (FIG. 20A). This key observation in live-sorted primarily cultured cells derived from clinical biospecimens provided support to use miR34a as a candidate gene replacement therapy. The introduction of miRNA34a into CD44^{hi} cells resulted in reduced soft agar colony formation (FIG. 20A), and the introduction of the anti-miRNA34a into CD44^{lo} cells led to an increased number and larger size of colonies in soft agar (FIG. 20B).

The CD44^{high} Tumorigenic Subsets in Lung Cancer Biospecimens are Enriched for Low miR-34a Expression

Cellular heterogeneity is an integral part of cancer development and progression that exhibit high phenotypic plasticity (including “de-differentiation” to primitive developmental states), and aggressive behavioral properties (including high tumorigenic potentials). Many biomarkers that are used to identify Cancer Stem Cells (CSC) can label cell subsets in an advanced clinical stage of lung cancer (malignant pleural effusions, or MPE). Thus, CSC-biomarkers are useful for live sorting functionally distinct cell subsets from individual tumors, which enables investigators to hone in on the molecular basis for functional heterogeneity.

In the data shown below, CD44^{hi} (CD44-high) cancer cell subsets displayed higher clonal, colony forming potential than CD44^{lo} cells (n=3) and are also tumorigenic (n=2/2) when transplanted in mouse xenograft model. The CD44^{hi} subsets, expressed different levels of embryonal (de-differentiation) markers or chromatin regulators. In archived lung cancer tissues, ALDH markers co-localize more with CD44 in squamous cell carcinoma (n=5/7) than Adeno Carcinoma (n=1/12). MPE cancer cells and a lung cancer cell line (NCI-H-2122) exhibited chromosomal abnormalities and 1p36 deletion (n=3/3). Since miR-34a maps to the 1p36 deletion site, low miR-34a expression levels were detected in these cells. The colony forming efficiency of CD44^{hi} cells, characteristic property of CSC, can be inhibited by mir-34a replacement in these samples. In addition the highly tumorigenic CD44^{hi} cells are enriched for cells in the G2 phase of cell cycle.

Materials and Methods:

Malignant Pleural Effusion (MPE) Collection, Processing and Cell Culture:

All subjects in the study underwent written informed consent by a process approved by the institutional review board (IRB) at the Veterans Affairs-Greater Los Angeles Healthcare System (VAGLAHS) and the study was approved by IRB-VAGLAHS. MPE specimens (M-1, M-2 and M-3) were collected from patients at Veterans Affairs-Greater Los Angeles Healthcare System (VAGLAHS). Cells are cultured in presence of 20-30% MPE (primary culture medium or PCM) as described previously (5). (Supplemental S-1 and S-2)

Control Established Cell Lines:

Two established cell lines GM05933 (normal fibroblast) and H2122 (lung cancer) were used in the study. The fibroblast cell line GM 05399 was obtained from the Coriell Institute for Medical Research (Camden, N.J.). The cell line was derived from a 1-year old Caucasian male. The cell line is maintained in our laboratory in Dulbecco's Modified Eagle's Medium (DMEM) in presence of 10% fetal bovine serum (FBS) (20). The H2122 lung adenocarcinaoma cell line was generated by Adi Gazdar from a malignant pleural effusion, and acquired from Ilona Linnoila and Herb Oie from the NCI. It was subsequently deposited into ATCC (NCI-H2122 [H2122] ATCC® CRL-5985™) (21). The cell line is maintained in our laboratory in RPMI-1640 medium in presence of 10% FBS (22, 23). Both the cell lines are publicly available.

Antibodies:

The following antibodies were used for flow cytometry FACS/Sort: Mouse anti-Human IgG2b CD44-FITC, (BD Biosciences #555478); FITC Mouse IgG2b κ Isotype control, (BD Biosciences #555742); PE-labeled mouse anti-human CD44, (BD Pharmingen #555479); PE Mouse Mouse IgG2b κ Isotype control, BD Biosciences 555743. Anti-CD166-FITC (Mouse monoclonal; IgG1 Setrotech #MCA 1926F, primary unlabeled anti-cMET (mouse IgG2a, Abcam #49210), anti-uPAR (mouse IgG, Santa Cruz Biotech #13522), Secondary antibody used for the study used were: Goat (Fab′)2 anti-Mouse IgG (H+L)-PE-Cy.5.5 (Caltag laboratories #M35018).

Immunohistochemistry (IHC):

Primary human lung cancer tissue (squamous cell carcinoma: SCC and adenocarcinoma: AC) or human lung control tissue (human normal alveolar and bronchiolar tissues) were obtained from the UCLA Department of Pathology core facility. Xenograft tumors derived from CD44^{hi} cells injected in NOD/SCID (IL2rγ^{null}) mice were surgically removed, cut into 0.3-0.5 mm pieces and fixed in ethanol (Fisher Scientific) or Z-fix (Anatech, Mich.). For IHC, sections 3-5 μm sections were cut and deparaffinized and processed for antigen retrieval (5) and stained for marker expression. Initially tissue sections were stained with single marker antibody staining (CD44 or ALDH). Once the conditions were optimized for single antigen staining dual antigen staining (CD44 and ALDH) of tissue sections was achieved. Paraffin-embedded tissue sections were deparaffinized and rehydrated. After antigen retrieval (10 mM sodium citrate buffer, PH 6.0 by steam 25 minutes) and blocking, endogenous peroxidases was quenched (3% H2O2 in 1% sodium azide with PBS, 30 minutes in room temperature). Slides were incubated with primary rabbit polyclonal antibody to ALDH1A1 (Abcom Inc. Cat#ab51028), overnight at 4° C. The slides were washed with PBS and incubated with EnVision+ System-HRP Labetted Polymer Anti-Rabbit (Dako Cat#K4003) for 30 minutes. The slides were incubated in DAB (Vector Peroxidaes Substrate Kit #SK-4100 with Nickel Sol) for 10-20 minutes and then the slides were washed 5 minutes 3 times with PBS. For double staining with CD44 (R & D Systems, mouse monoclonal IgG, Cat#BBA 10), slides were also incubated in the primary antiserum at room temperature for 1 hour, followed by the secondary antibody, Biotinylated-anti-mouse IgG (Vector Cat#9200), and then, ABC kit (Vector Cat#AK-5000) and Vector Red Alkaline Phosphatease Substrate Kit I (Vector Cat#SK-5100), developed for 20 minutes. Sections were counter-stained with Harris' hematoxylin, dehydrated in graded alcohol, cleared in xylene and mounted on glass slides with cover slip. The stained sections were examined under a microscope (Leica-Leitz DMRBE or Olympus 1X71) and positive or dual antigen expressing areas determined by pathologists at UCLA.

Cytology and Flow cytometry (FACS):

Photomicrographs were taken using the Leica-Leitz DMRBE microscope mounted with a CCD camera and FACS analysis was done using the Becton Dickinson FACSCalibur Analytic Flow Cytometer (5). Cell sorting was performed using the Becton Dickinson FACSVantage SE Sorting Flow Cytometer at the UCLA-JCCC Flow-cytometry core facility.

Reverse transcriptase—PCR (RT-PCR) Analysis of Gene Expression:

The primary samples were first sorted into CD44^{hi} and CD44^{lo} populations. The cells were collected and RNA was extracted using Trizol and Fast Track 2.0 mRNA isolation kit (Invitrogen Inc., Carlsbad, Calif.) and was reverse transcribed using RT kit (5). The samples were used for PCR for the amplification of Bmi1, hTERT, SUZ12, EZH2, and Oct4 genes. The following primers were used: Bmi1 Forward-5′ AATCTAAGGAGGAGGTGA 3′, (SEQ ID NO:1); Reverse-5′ CAAACAAGAAGAGGTGGA 3′, (SEQ ID NO:2); hTERT Forward-5′ GGAATTCTGGAGCTGCTTGGGAACCA 3′, (SEQ ID NO:3); and Reverse-5′ CGTCTAGAGCCGGACACTCAGCCT-TCA 3′, (SEQ ID NO:4); SUZ12 Forward-5′ GATAAAAACAGGCGCTTA-CAGCTT 3′, (SEQ ID NO:5); and Reverse-5′ AGGTCCCT-GAGAAAATGTTTCGA 3′, (SEQ ID NO:6); EZH2 Forward-5′ TTGTTGGCGGAAGCGTGTAAAATC 3′, (SEQ ID NO:7); and Reverse-5′ TCCCTAGTCCCGCGC-AATGAGC-3′, (SEQ ID NO:8); and Oct4 Forward-5′ CAACTCCGATGGGGCCCT 3′, (SEQ ID NO:9); and Reverse-5′ CTTCAGGAGCTTGGCAAATTG 3′ (SEQ ID NO:10). The conditions for amplifications of different genes have been described previously. PCR products were separated by 8% gels (TBE, 50 mM Tris borate pH 8.0, 1 mM EDTA) followed by Ethedium Bromide staining Gels were analyzed using the Kodak 1D software.

Colony Formation Efficiency Assay:

In vitro colony-formation assays were done as described (Patrawala L, et al. Hierarchical organization of prostate cancer cells in xenograft tumors: the CD44+alpha2beta1+ cell population is enriched in tumor-initiating cells. Cancer Res. 2007 Jul. 15; 67(14):6796-805. Erratum in: Cancer Res. 2007 Sep. 15; 67(18):8973.)). Sorted CD44^{hi} and CD44^{lo} cells were plated at clonal density (100-500 cells/well) in six well tissue culture dishes in triplicates. Holoclones with >20 cells were counted at the end of 10 days of culture. The results are expressed as percentage cloning efficiency.

Spheroid Formation in Soft Agar Assay:

Sorted CD44^{hi} and CD44^{lo} cells were plated at 1000 cells/well in triplicates in six-well culture plates containing 0.35% top agar layered over 0.5% base agar (DNA Grade) containing PCM. Colonies were counted at 3 weeks post plating, results represent mean from three independent experiments.

Tumorigenicity in NOD/SCID (IL2rγ^{null}) Mice:

All mice work related protocol for the study was approved by the Institutional Animal Care and Use Committee at UCLA/VAGLAHS. CD44^{hi} and CD44^{lo} cells were sorted by FACS and injected at different cell doses (300/mouse, 3000/mouse and 30000/mouse; 3 mice/group) at the right and left flank respectively in NOD/SCID (IL2γ^{null}) mice in 100 μl of saline. Mice were monitored for tumor growth at both the flanks. Results are represented as group averages of tumor volume, as described (24).

miR-34a Transfection Studies:

To analyze the effects that miR-34a has on colony formation efficiency in soft agar assay the CD44^{hi} cells were transiently transfected with either miR-34a (AM17100, Applied Biosystem/Ambion) or the negative control (scrambled) oligoneucleotide. Similarly CD44^{lo} cells were transiently transfected with either anti-miR-34a inhibitor (#AM17000, Applied Biosystem/Ambion) or negative control anti-miR oligoneucleotide (#AM17010, Applied Biosystem/Ambion). The transfection was carried out with CD44^{hi} or CD44^{lo} cells using Lipofectamin 2000 (Invitrogen) in 6 well plates with 50,000 cells/well with 100 pmol of miR, anti-miR and control scrambled/oligonucleotides. After 2 days of transfection the cells were collected and assayed for soft agar colony forming efficiency as described above.

Fluorescent In Situ Hybridization (FISH) Analysis of MPE Samples:

FISH studies were performed according to established protocol (Srivatsan E S, et al. Interstitial deletion of 11q13 sequences in HeLa cells. Genes Chromosomes Cancer. 2000 October; 29(2):157-65.). LSI 1p36 probe was labeled with spectrum orange and LSI 1q25 probe was labeled with spectrum green and hybridized to metaphase spreads as previously described (Srivatsan E S and Winokur S T, et al. The evolutionary distribution and structural organization of the homeobox-containing repeat D4Z4 indicates a functional role for the ancestral copy in the FSHD region. Hum Mol Genet. 1996 October; 5(10):1567-75. Erratum in: Hum Mol Genet. 1997 March; 6(3):502). Briefly, metaphase spreads were prepared by standard cytogenetic procedures. Labeled probes were hybridized and washes were performed under identical conditions of stringency. Slides were hybridized at 37° C. overnight with 1-4 ng of the probe, 50% formamide, 10% dextran, 2×SSC, and 50 ng Cot 1 DNA to suppress repetitive sequences. Metaphase chromosomes were counterstained with 4,6-diamidino 2-phenylindole (DAPI) in Vectashield solution (Vector Laboratories Inc., Burlingame, Calif.). Karyotyping of chromosomes were performed according to established protocols.

Reverse Transcriptase—Quantitative PCR (RT-qPCR) Detection of mir-34a in MPE Samples:

Total RNA was isolated from samples using TRIzol. miR-34a was measured by Step One Plus Real-time PCR system (Applied Bio systems, CA) by using Taq-Man MicroRNA Assays (Applied Biosystems, Foster City, Calif.) and normalized by RNU48 levels. 3 μl of 20 ng/μl of total RNA was used to perform Reverse Transcriptase (RT) reaction (30 min at 16° C., 30 min at 42° C., 5 min at 85° C.) using 10 mM dNTPs, MultiscribeRT enzyme, 10×RT buffer, RNase inhibitor, Taqman RT primer and water in total reaction volume of 15 μl. For qPCR, 10 μl of 2× Taqman universal PCR master mix (No AmpErase UNG from ABI), 7 μl of water, 1 μl of Taqman primer (miR-34a and RNU48) and 2 μl for cDNA for each reaction was used, following amplification protocol (10 min at 95° C., 15 sec for 95° C., 60 sec at 60° C. for 40 cycles) using Step One Plus Real-time PCR system (Applied Biosystems, CA).

Surface Marker Labeling and Cell Cycle Analysis:

Cells were stained with CD44-FITC and PI (Propidium Iodide) for cell cycle analysis (modified from UCLA/Flow-cytometry core facility protocol). Briefly, 1×10⁶ single cell suspension was washed with PBS/2% PCM, pelleted, and labeled with mouse anti-Human IgG2b CD44-FITC antibody (BD Biosciences #555478) for 45 min at room temperature in dark, control antibody was used as negative control. The samples were re-suspended in 1 ml of buffer containing 10 micrograms/ml of PI and 11.25 Kunitz units of RNase and incubate for at least 30 min at 4° C. in the dark and analyzed on the flow cytometer within 30 min of PI staining.

Statistical Analysis:

Data are represented as mean±SD and were analyzed with two-sided t test by EXCEL and repeated measures analysis of variance (ANOVA) was used for comparison among groups by SAS 9.3. A P value<0.05 was considered statistically significant.

Results

CD44 Expression Profile of MPE Derived Tumor Cells

MPE-tumor cells can be isolated and expanded in short term primary cultures in presence of MPE fluid and autologous non-tumor cells. Heterogeneous populations, including candidate CSC, were present in the MPE-tumor population, as reflected by the variable expression of CSC-biomarkers: c-MET, uPAR, MDR1, CD166, CD44, and ALDH. Thus, in addition to intratumoral morphological heterogeneity, there are differences in the surface CD44 labeling intensities, and these differences can be exploited to segregate cell subsets.

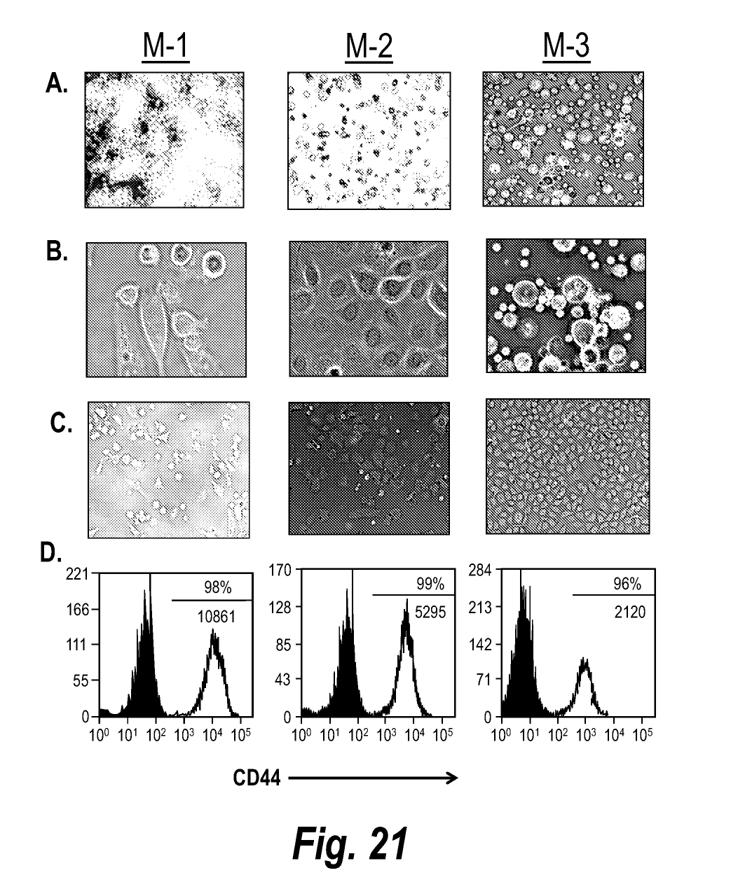

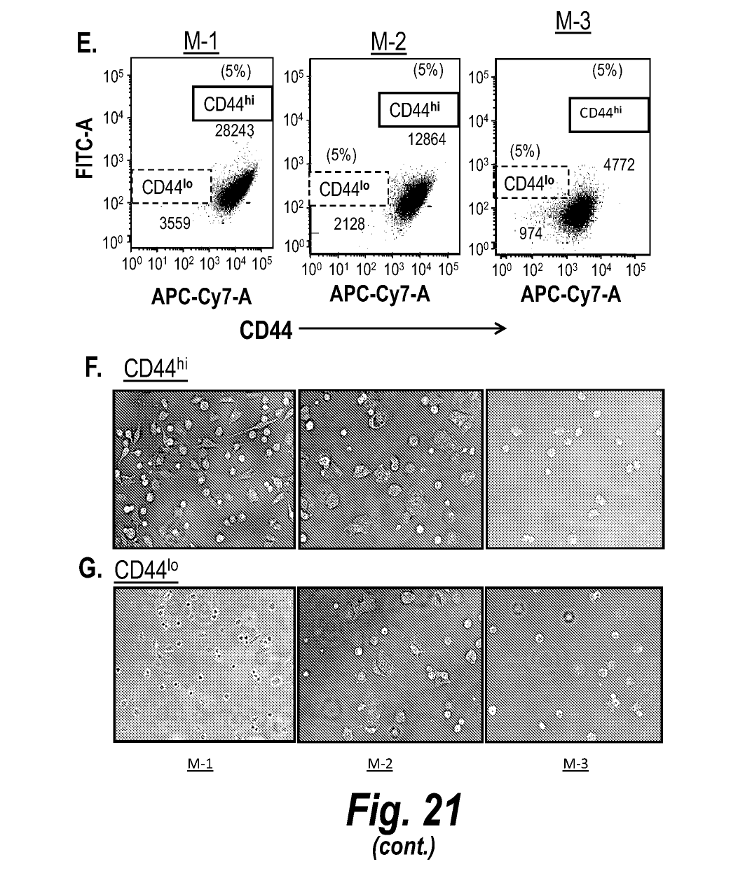

The primary cultures from three different MPE-samples (M-1, M-2 and M-3), contained morphological variants (flat, oval and rounded shapes) by light microscopy (FIG. 21A, B). By the 4^{th} week of culture, the adherent tumor cells display a more homogeneous morphology pattern in culture (FIG. 21C). Cultured cells uniformly express CD44 in all three tumor samples (FIG. 21D), but the labeling intensity is highly variable both between and within the same sample. Thus, compared to cells labeled with secondary antibody alone, the samples are 96%, 99% and 98% positive for CD44; however, the Mean Fluorescence Intensities (MFIs) of CD44 labeling are 10861, 5295 and 2120 respectively. Thus, the surface labeling intensity of CD44 expression may vary from 2 to 5 fold among tumor samples, and there is typically a large variance in average surface CD44 labeling within individual samples.

Absence of Morphological Differences Between CD44^{hi} and CD44^{lo} Cells

MPE-primary cultures acquire a more homogenous morphological pattern of growth over time. To determine if subtle differences in culture morphology could distinguish the CD44^{hi} from CD44^{lo} cultures, the M-1, M-2 and M-3 samples were labeled with anti-CD44 antibody and sorted by FACS, with gates set at 5% of cells at the high CD44 marker and low CD44 marker expression (FIG. 21E). The purity of the CD44^{hi} and CD44^{lo} cells were ≧98%, as revealed by post sort analysis (data not shown). The sorted cells CD44^{hi} (FIG. 21F) and CD44^{lo} (FIG. 21G) were washed and plated out in PCM for 2-3 days to evaluate their morphological differences. These studies suggest that there is no distinguishing difference in culture morphology associated with surface CD44 expression.

CD44^{hi} Cells Show High Colony Forming Ability

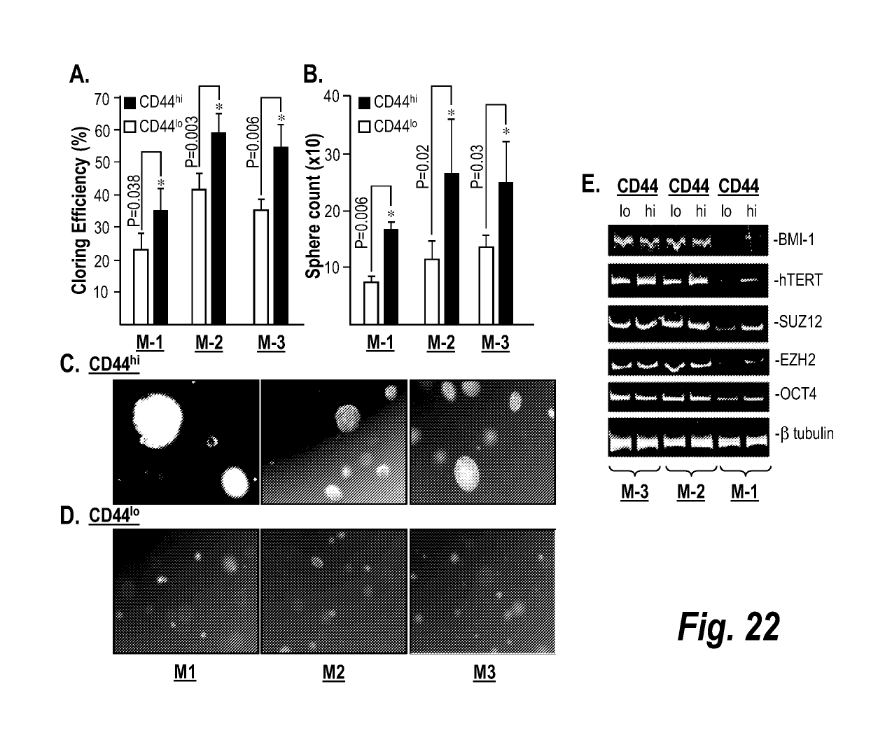

To investigate whether the CD44^{hi} cells were functionally different from the CD44^{lo} cells in colony forming efficiency, these subsets from the three samples (M-1, M-2 and M-3) were sorted and cultured. 100-500 cells of CD44^{hi} or CD44^{lo} cells were plated in individual wells of 12-well plates. Although significant differences in initial plating efficiency were not detected, CD44^{hi} cells were more competent at forming holoclones than the CD44^{lo} cells (t test and ANOVA: P<0.05) (FIG. 22A). Thus, an intrinsic biological difference between CD44^{hi} and CD44^{lo} cells seems to an inherent differential competency in forming holoclones.

CD44^{hi} Cells Show High Spheroid Forming Ability in Soft Agar Cultures

Another surrogate measure commonly used to characterize CSC is a differential competency at forming “anchorage independent” colonies in soft agar. CD44^{hi} and CD44^{lo} cells from samples (M-A-1, M-10-26 and M-8-15) were evaluated by plating the sorted cells in agarose supplemented with PCM. The CD44^{hi} cells from all three samples uniformly exhibit higher spheroid formation efficiency than the CD44^{lo} cells (t test and ANOVA: P<0.05) (FIG. 22B). The more robust CD44^{hi} colonies are also qualitatively distinguishable from vestigial colonies formed by CD44^{lo} cells (FIG. 22C versus 22D). Thus, CD44^{hi} cells possessed greater competency at forming colonies in soft agar than the CD44^{lo} cells derived from the same lung cancer biospecimen.

CD44^{hi} Cells Variably Display Molecular Features that Characterize CSC

Markers that characterize candidate CSC (hTERT, SUZ12, OCT-4 expression etc.) were evident in cell pellets isolated from the MPE samples; thus, CSC are a likely component of the MPE-tumor mix. Such CSC markers are variably comprised of embryonal or polycomb protein components and their expression may predict the labeling of cell subsets that possess high tumorigenic or colony forming potentials. To determine if these candidate CSC markers were limited to specific CD44 sorted subsets, the CD44^{hi} and CD44^{lo} cell subsets were screened for differential mRNA expression. RT-PCR amplification of BMI-1, hTERT, SUZ-12, EZH2 and OCT4 was performed. Indexed to beta-tubulin mRNA, there was a marked variability in the expression of these markers within the CD44-sorted cell subsets (FIG. 22E). For example, BMI-1 and hTERT mRNA is more highly expressed in CD44^{hi} cells than the CD44^{lo} cells in sample M-3 and M-2 respectively. Only in sample M-1, expected distributions of CSC markers (high BMI, hTERT, SUZ12, EZH2 and OCT-4) are evident in CD44^{hi} cells than the CD44^{lo} cells.

These results indicated that 1) molecular markers that encode for modifiers of chromatin structure or embryonal genes may be present in both highly tumorigenic and non-tumorigenic subsets of individual lung cancer cell populations, and 2) that there was marked variability in the differential expression of these candidate “CSC-biomarkers” in lung cancer biospecimens.

CD44^{hi} Cells Form Tumors in NOD/SCID (IL2Rγ^{null}) Mice

The CD44^{hi} and CD44^{lo} cell subsets from individual tumor cell populations consistently displayed differences in adherent holoclone and soft agar colony formation. A key experimental measure of “CSC”, however, is by the demonstration of higher tumorigenic potential in mouse models. It has been shown that NOD/SCID(IL2rγ^{null} mice were a sensitive model to evaluate for highly tumorigenic CSC-behavioral phenotypes. To corroborate observed differences in colony forming and spheroid forming abilities of CD44^{hi} vs CD44^{lo} cells with in vivo tumorigenensis, we investigated their ability to form tumors in NOD/SCID (IL2rγ^{null}) mice.

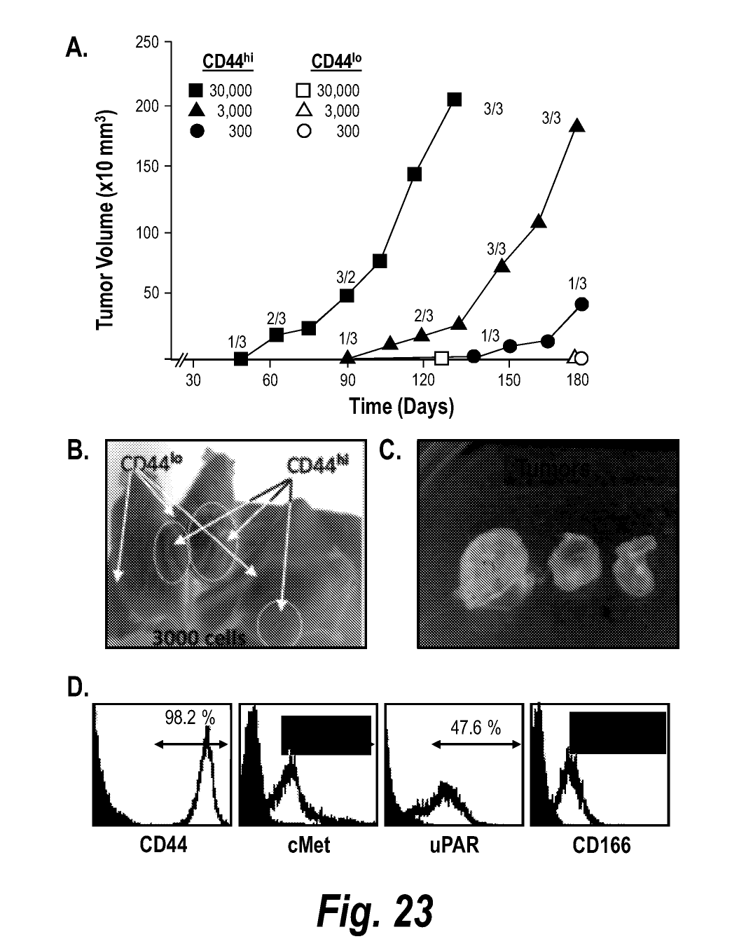

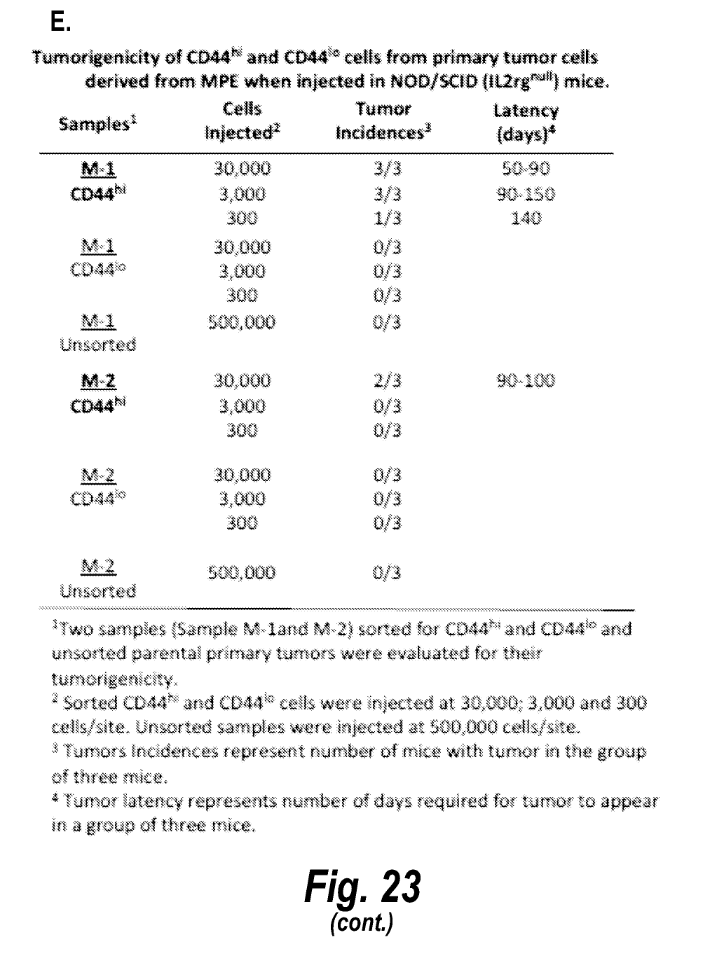

Limiting dilutions (30,000; 3,000; 300) of sorted CD44^{hi} and CD44^{lo} cells from M-1 and M-2 MPEs were injected into the right and left flanks respectively of NOD/SCID (IL2rγ^{null}) mice. CD44^{hi} tumor cells of the M-1 sample formed tumors in 3/3 mice at both 30,000 and 3,000 injected cell doses, and in one of 3 mice injected with 300 tumor cells (FIG. 23 A, B, C). The latency period of tumors was 50-90 days, 90-150 days and 150 days, for 30,000; 3,000 and 300 CD44^{hi} cells respectively (FIG. 23E). Thus, the kinetics of tumor formation by the highly tumorigenic CD44^{hi} cells was dose-dependent. The CD44^{hi} tumor cells from sample M-2 generated tumors in 2 of 3 mice at 30,000 tumor cells with a latency period of 90-100 days, a higher latency period than observed in CD44^{hi} cells of sample M-1 (FIG. 23E). Thus, although CD44^{hi} cells consistently display higher tumorigenic potentials than CD44^{lo} cells of the same specimen, individual tumor specimens may display different growth kinetics in the evaluation of CSC properties in behavioral bioassays.

Notably, the CD44^{lo} cells from either primary culture did not form tumors in the left flanks of the mice during the entire monitoring interval (FIGS. 23 B and E). Moreover, tumor formation was not observed with the injection of 5×10⁵ unsorted cells, even though this population presumably contained ˜5-10% (or 25,000-50,000) CD44^{hi} cells. This interesting observation suggests that CD44^{hi} cells may be exposed to inhibitory influences towards tumor growth by cells that have a lower intensity surface CD44 expression in the same tumor population.

To test whether implanted CD44^{hi} cells contributed to heterogeneous tumors (suggestive of multipotent differentiation), engrafted tumors generated from CD44^{hi} from M-1 cells were extirpated, digested, and cell surface marker analysis was performed by FACS on single cell suspensions. The tumor cells remained highly positive for the CD44 marker, with 98.2% of cells staining positive, although the CD44-MFI was even higher than the originally implanted cells. Heterogeneity amongst cells was evidenced by the variable expression of other commonly associated CSC biomarkers (FIG. 23D), [cMET (40.4%), uPAR (47.6%) and CD166 (27.4%)]. Cells bearing these markers were also previously detected in the primary MPE biospecimen samples, at varying fractions. Together, these results indicate that CD44^{hi} cells derived from MPE are not only more tumorigenic than the CD44^{lo} cells, but that the CD44^{hi} cells are also capable of generating tumors with heterogeneous marker profiles, similar to those found in the primary MPE samples.

CD44 and ALDH Expression in Implanted Xenografts Resemble Expression of these Markers in Archived Human Lung Cancer Pathology Specimens

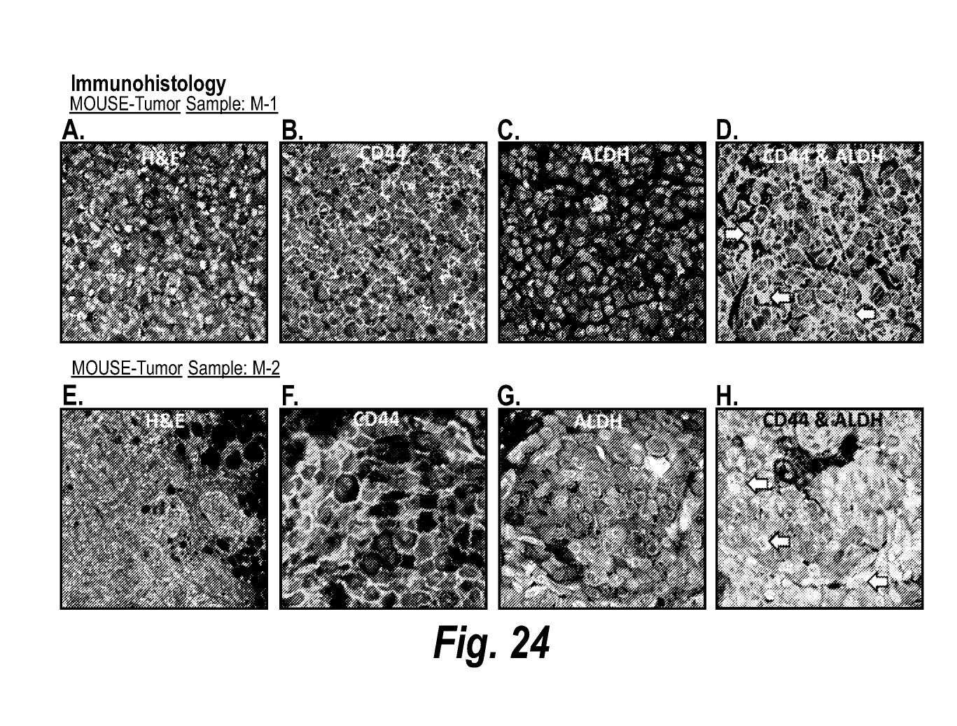

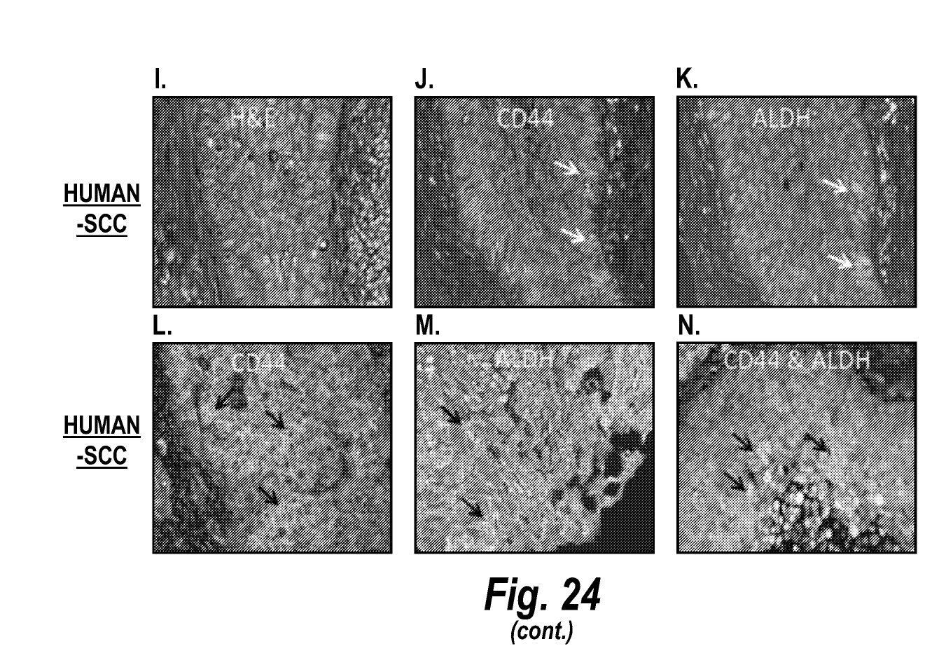

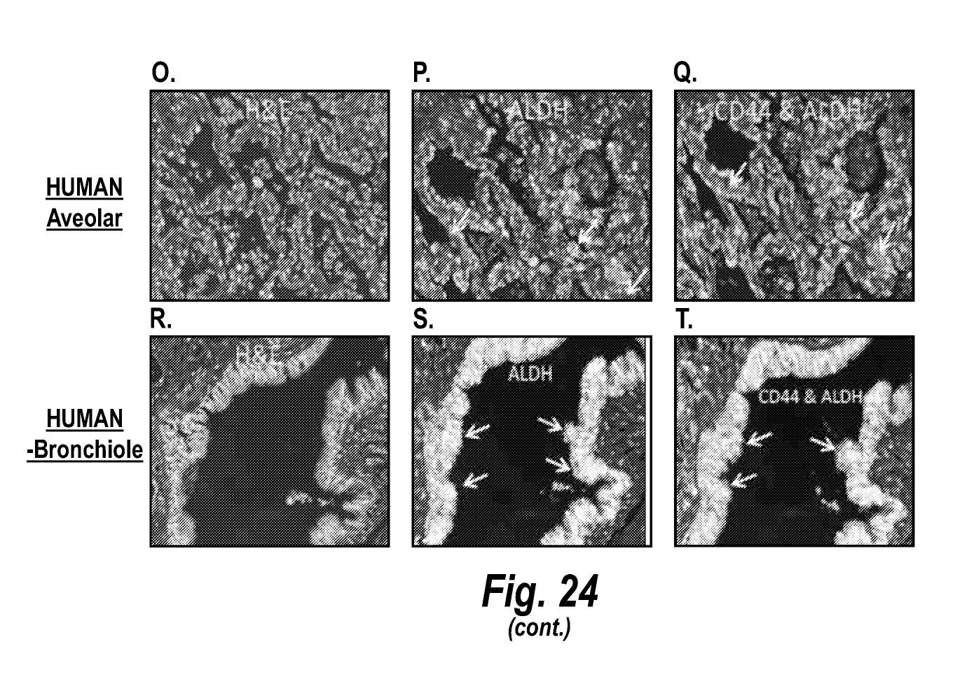

CD44^{hi} cells in MPE primary cultures contain cell fractions with high ALDH expression (i.e., the CD44^{hi}/ALDH^{hi} surface phenotype). Using immunohistochemistry, we observed variable expression of CD44 and ALDH markers in the mouse xenograft tumors generated from CD44^{hi} cells. Pathological and marker expression patterns in xenografts compare favorably to archived human lung cancer, and to tumor-adjacent human normal alveolar and normal human bronchiolar tissues. H&E sections of M-1 and M-2 CD44^{hi} xenografts (FIG. 24 A-H) corroborate the original pathological diagnoses of large cell lung cancer and lung SCC respectively (FIG. 24 I-L). Consistent with flow cytometry data, CD44 labeling is evident on the majority of cells. However, intra-tumoral variation of CD44 expression is clearly evident (FIG. 24 B, F), again consistent with the flow cytometry profile. Similarly when the xenograft sections were labeled for ALDH expression, some cells showed higher expression than other tumor cell populations (FIG. 24 C, G). When co-expression of CD44 and ALDH was examined by dual marker staining of xenograft tumor sections, there was tumor to tumor variability in the co-localization of these markers (FIG. 24 D, H).

These labeling patterns were representative of resected human pathology specimens, as evidenced by the morphology and immunohistopathology expression patterns observed in archived samples (FIGS. 24 I-J and L-M), which show CD44 (FIG. 24 J, L), ALDH (FIG. 24 K, M) and dual staining expression pattern of CD44 and ALDH (FIG. 24 N). Arrows in the figure point to high expression of respective markers in SCC tissue sections (FIG. 24 J-M). Dual expression of CD44 and ALDH in SCC tissue sections may be more intense toward the center of tumor nodules (FIG. 24 N).

To determine if such labeling was restricted to the neoplastic tissue, tumor adjacent normal lung alveolar (FIG. 24 O-Q) and bronchiolar (FIG. 24 R-T) tissues were also examined. The photomicrographs suggest that high expression of ALDH (FIG. 24 P), and co-expression ALDH and CD44 (FIG. 24 Q) was evident in histologically normal alveolar tissues as well, where representative H& E photomicrographs of normal bronchiolar tissue shows the characteristic presence of ciliated and goblet cells (FIG. 24 R). Foci of ciliated and goblet cells highly express CD44 or both CD44 and ALDH in this anatomical location (FIG. 24 S, T). Since CD44 and ALDH markers are also co-expressed in normal lung tissues, the presence of these markers per se may not distinguish neoplastic from non-neoplastic tissues.

To determine if we could identify a relationship between CD44/ALDH expression and histopathological subtypes of lung cancer, tissue sections were evaluated for CD44, ALDH and co expression of CD44 and ALDH. CD44 and ALDH are commonly expressed in all lung tumor samples, both with respect to fractions of cell labeling, and intensity of labeling (Table 8).

TABLE 8
CD44 and ALDH expression pattern in Squamous Cell Carcinoma
(SCC) and Adenocarcinoma (AC) of the lung.

| | | | CD44 Staining | | ALDH Staining | | |
|---|---|---|---|---|---|---|---|
| | Tumor | | | Inten- | | Inten- | Co-local- |
| Case | Type | Grade | Amount | sity | Amount | sity | ization |
| S-46 | SCC | 3+ | 4+ | 3+ | 1+ | 1+ | Y |
| S-44 | SCC | 2+ | 1+ | 3+ | 2+ | 2+ | Y |
| 1-57 | SCC | 3+ | 4+ | 3+ | 3+ | 2+ | Y |
| S-24 | SCC | 3+ | 4+ | 3+ | 4+ | 3+ | Y |
| S-34 | SCC | 3+ | 4+ | 3+ | 1+ | 1+ | Y |
| 1-2T | SCC | 3+ | 0+ | 0+ | 1+ | 2+ | NA |
| 1-76 | SCC | 2+ | 4+ | 3+ | 0+ | 0+ | NA |
| 9-69 | AC | 3+ | 0+ | 0+ | 0+ | 0+ | NA |
| 1-68 | AC | 2+ | 0+ | 0+ | 0+ | 0+ | NA |
| 8-94 | AC | 3+ | 1+ | 3+ | 0+ | 0+ | NA |
| S-06 | AC | 1+ | 4+ | 3+ | 0+ | 0+ | NA |
| 1-70 | AC | 2+ | 0+ | 0+ | 2+ | 2+ | NA |
| 1-2T | AC | 1+ | 0+ | 0+ | 0+ | 0+ | NA |
| 1-08 | AC | 3+ | 0+ | 0+ | 3+ | 3+ | NA |
| S-57 | AC | 2+ | 2+ | 3+ | 0+ | 0+ | NA |
| S-42 | AC | 1+ | 0+ | 0+ | 0+ | 0+ | NA |
| 1-18 | AC | 3+ | 0+ | 0+ | 0+ | 0+ | NA |
| 9-79 | AC | 2+ | 1+ | 2+ | 2+ | 2+ | N |
| S-35 | AC | 1+ | 4+ | 3+ | 1+ | 1+ | Y |

Tumor Type
SCC = Squamous cell carcinoma
AC = Adenocarcinoma
Grade
1+ = Well differentiated
2+ = Moderately differentiated
3+ = Poorly differentiated
Amount of Staining
0+ = <5% of cells
1+ = 6 to 24% of cells
2+ = 25 to 49% of cells
3+ = 50 to 74% of cells
4+ = 75% or greater of cells
Intensity of Staining
0+ = no staining
1+ = weekly positive
2+ = moderately positive
3+ = strongly positive
Co-localization
N = No co-localization
Y = co-localization identified
NA = Not Applicable

The data suggest that SCC express higher levels (4+/3+) of CD44 and ALDH than adenocarcinoma, and co localization of these markers (the CD44^{hi}/ALDH^{hi} surface phenotype) is also easier to identify in SCC (n=5/7) than in adenocarcinoma (n=1/12).

Rearrangement of Chromosome 1p36 and Reduced Expression of miR-34a in CD44^{hi} Cells

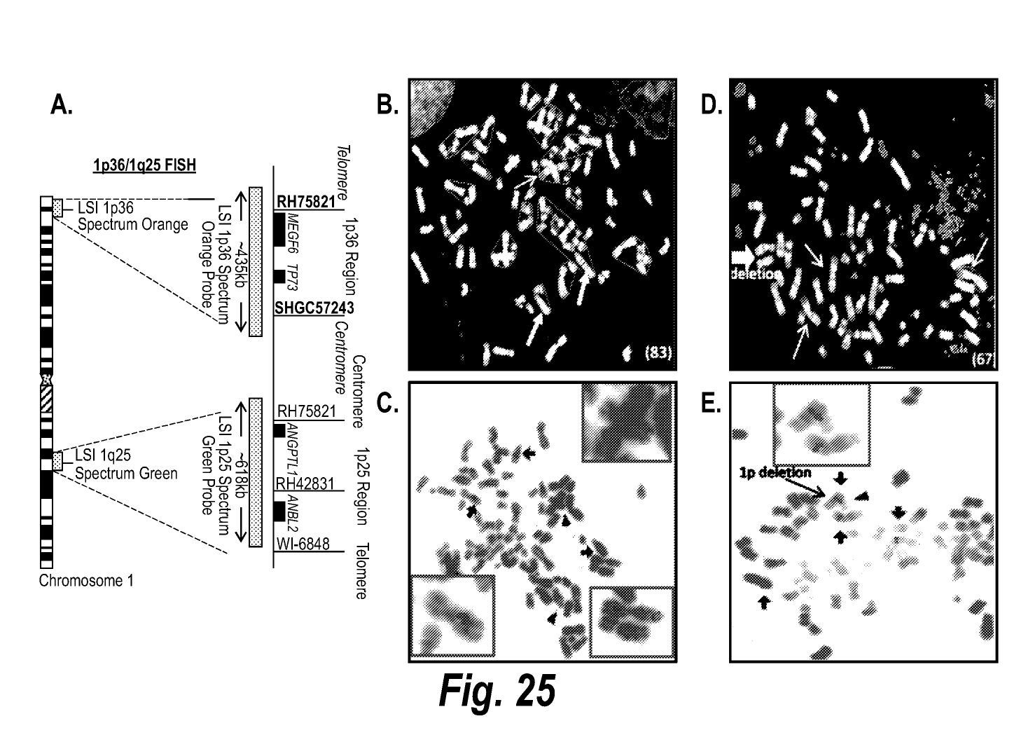

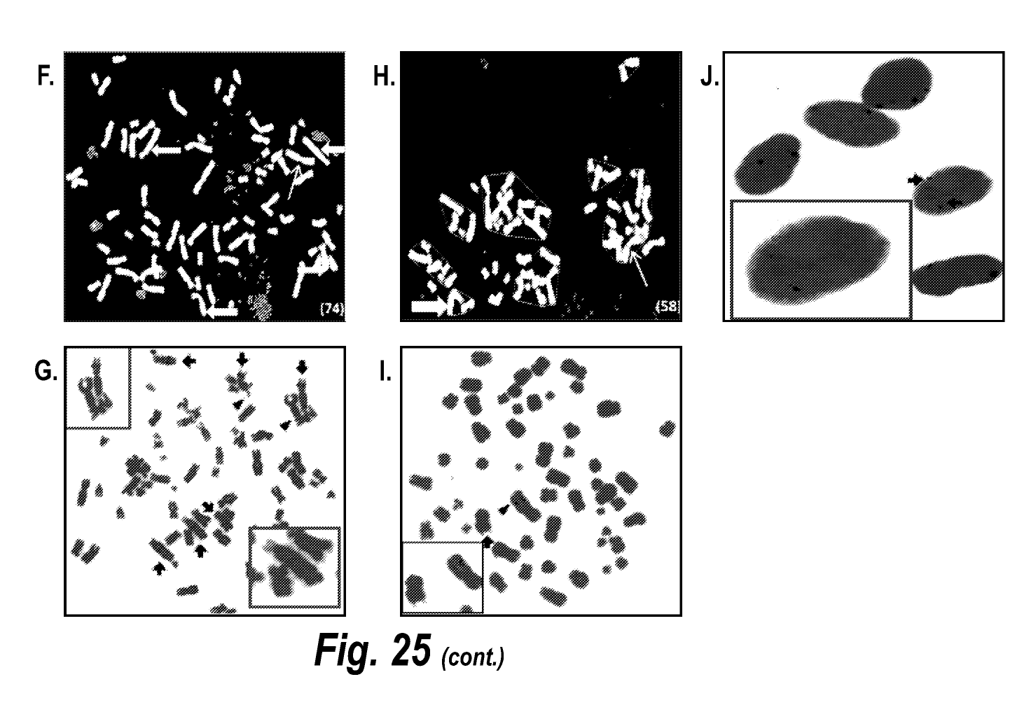

Abnormal chromosomal numbers, and both hyper- and aneuploidy are common in lung cancer. It is not clear whether such chromosomal changes are associated with the tumorigenic potential of cancer cells. To investigate a possible association, karyotype analysis was performed on the three MPE samples. Normal fibroblast GM 05399 and the lung cancer cell line NCI-H2122 were used as controls to represent non tumorigenic and immortalized tumor cell models. All three MPE samples M-1, M-2 and M-3 showed extensive chromosomal changes with hyperdiploid number of chromosomes 83, 67, and 74 respectively (FIG. 25B, D, F). Meanwhile, the normal fibroblast contained 46 chromosomes; the cell line NCI-H2122 contained 58 chromosomes (FIG. 25H). MPE cells uniformly contained translocations and deletions, and rearrangements at chromosomal region 1p, a common site of rearrangements seen in lung cancers. A FISH analysis was carried out using a 1p36 (orange) probe and a control 1q25 (green) probe to detect specific 1p changes (FIG. 25A). Sample M-1 has 3 copies of chromosome 1 (↑), of which 2 copies (Δ) are rearranged at 1p and 1q (FIG. 25C). The sample M-2 exhibits 4 Chromosome is (↑) (3 with intact 1p/1q and 1 with 1p deletion (Δ)) (FIG. 25E). The third sample M-3 has 2 copies of 1p (Δ) and 6 (↑) copies of 1q (consistent with 1p deletion) (FIG. 25E).

The immortalized MPE-derived lung cancer cell line (NCI-H2122) also displays an abnormal karyotype with hyperploidy (FIG. 25H). NCI-H2122 has 2 copies (↑ and Δ) of 1p/1q but one 1p is rearranged with additional material of unknown origin at 1p terminal region (Δ) (FIG. 25I). By contrast, the normal diploid human fibroblast GM 05399 cells show normal distribution of two copies of 1p/1q (↑) (FIG. 25J).

Thus, we detected Loss of Heterozygosity (LOH) at 1p36 in two MPE samples and rearrangements of both 1p/1q regions in the third MPE sample. Cell line H-2122 contained one normal chromosome 1 and unbalanced translocation of unknown origin at 1p36 consistent with deletion of 1p. The observations suggested that 1p36 deletion could result in the inactivation of a tumor suppressor gene. A bioinformatics search identified candidates, including the code for miR-34a that mapped to this locus.

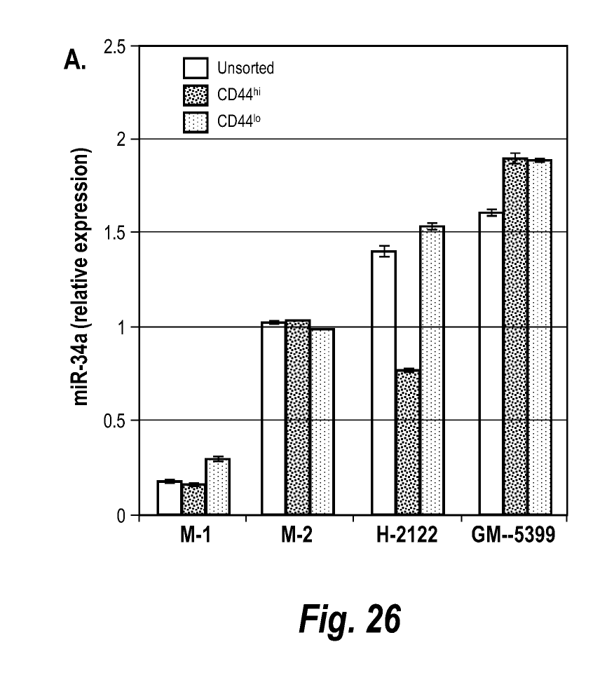

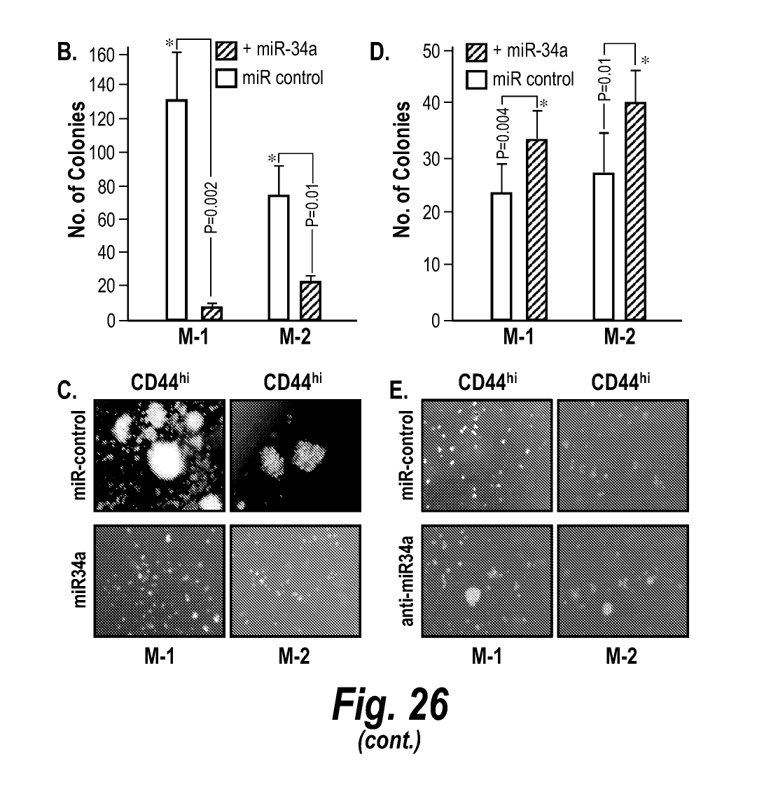

Since expression of miR-34a may contribute to the different biological properties of CD44^{hi} versus CD44^{lo} cells in individual tumors, its expression levels was evaluated in CD44^{hi}, CD44^{lo} and unsorted total cell populations in fractionated MPE-biospecimens (FIG. 26A). The expression of small nucleolar RNA-RNU48 was used as a reference for gene expression in this assay and miR-34a results were normalized with the RNU48 expression. Although the expression of the small nucleolar RNA-RNU48 may itself be dysregulated in cancer, our study demonstrated a similar basal expression pattern across the sample sets. On RT-qPCR analyses, however, there was no significant difference in miR-34a expression in the CD44^{hi} and CD44^{lo} subsets of the MPE sample M-2; the expression in this sample was similar to that of the control fibroblasts. In contrast, CD44^{hi} cells have significantly lower level of miR-34a than the CD44^{lo} cells in sample M-1, as well as in the immortalized cell line NCI-H2122. These data suggest that loss of miR34a may contribute to aggressive biological properties and high tumorigenic potentials in some lung cancers.

Soft Agar Colony Formation by CD44^{hi} Cells is Correlated with the Decreased Expression of miR-34a in Individual Lung Cancers

To determine whether the decreased expression of miR-34a could be directly associated with an aggressive phenotype in some lung cancers, we compared colony forming ability and tumorigenic potentials of CD44^{hi} tumor cells with their miR-34a expression. The loss of miR-34a in these samples directly correlated with a competency at high colony formation. Thus, CD44^{hi} cells with the lowest miR-34a expression formed a higher number and larger colonies, while CD44^{lo} cells with higher miR-34a expression formed smaller number of vestigial colonies (FIG. 22). Fibroblasts, with high miR-34a expression, failed to form colonies in soft agar.

To further assess the role of miR-34a towards mediating a biological effect in tumor cells subsets, CD44^{hi} and CD44^{lo} cell populations were transfected with miR-34a or anti-miR-34a, and colony formation was assayed. Introduction of miR-34a into CD44^{hi} cells resulted in 80-95% reduction of soft agar colonies (t test: P=0.01-0.002) (FIG. 26B, C). As expected, introduction of anti-miR-34a into CD44^{lo} led to increased number of soft agar colonies (t test: P=0.04-0.01) (FIG. 26D, E). The results were significant by t test analysis as indicated in FIGS. 26B and 26D. Though the differences were significant, however, by ANOVA test the P values were 0.112 (FIG. 26B) and 0.125 (FIG. 26D), indicating that either the variability within the two samples were greater or the sample numbers were few to be significant by ANOVA analysis. However, miR-34a clearly plays an important role in tumor growth suppression; the loss of miR-34a expression is evident in aggressive (CD44^{hi} subsets) of individual cancers, and this loss directly contributes to the development of a highly tumorigenic phenotype.

CD44^{hi} Cells Display Extended G2 Phase Cell Cycle

It is believed that CSCs remain in quiescent state and cycle slower through the cell cycle; these are properties resembling normal stem cells. The cell cycle phase of the CD44^{hi} cells that show higher tumorigenic potential was evaluated by FACS.

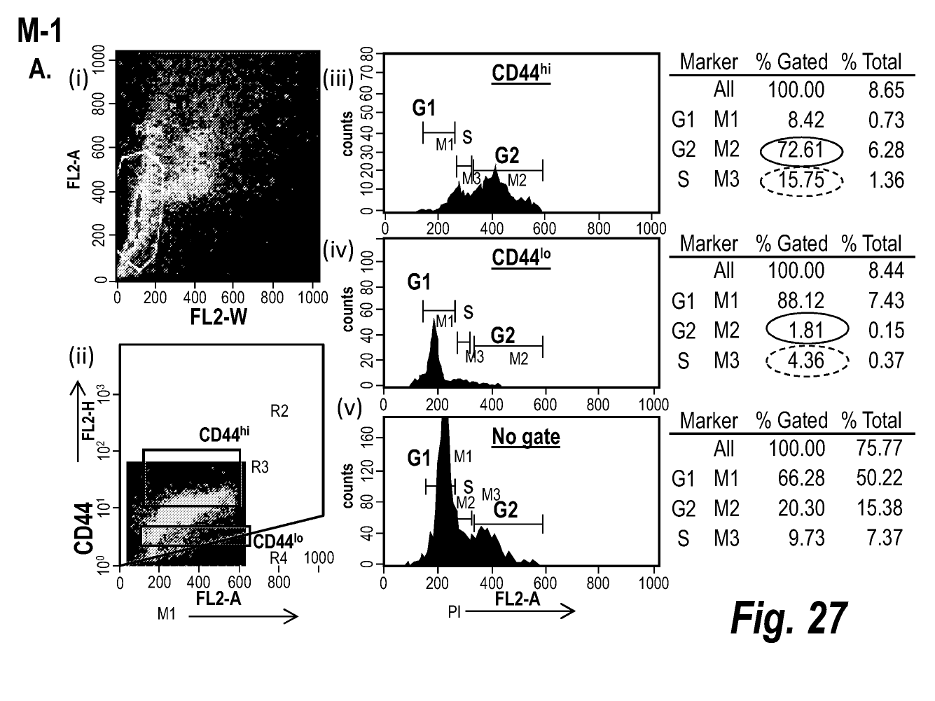

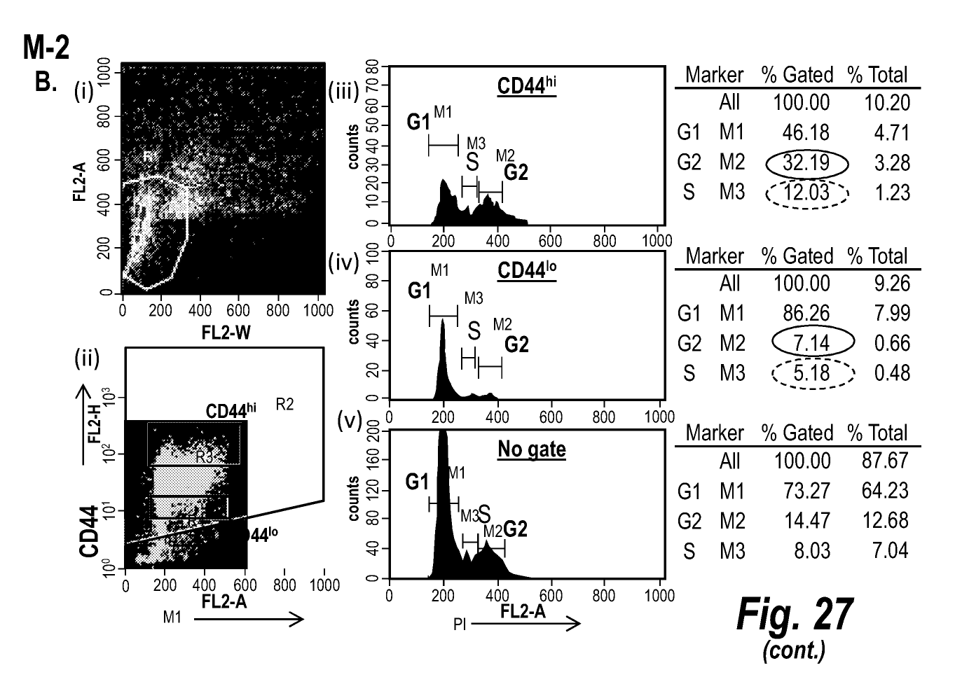

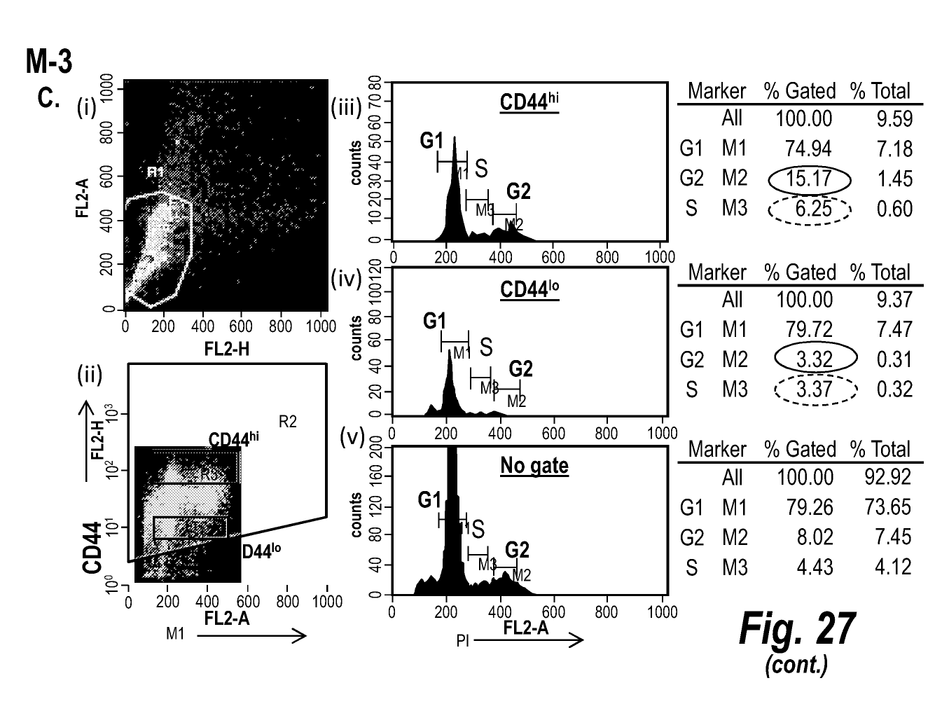

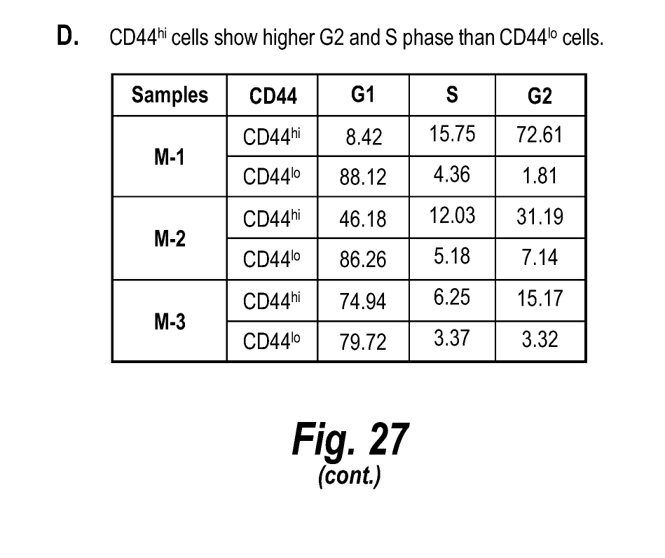

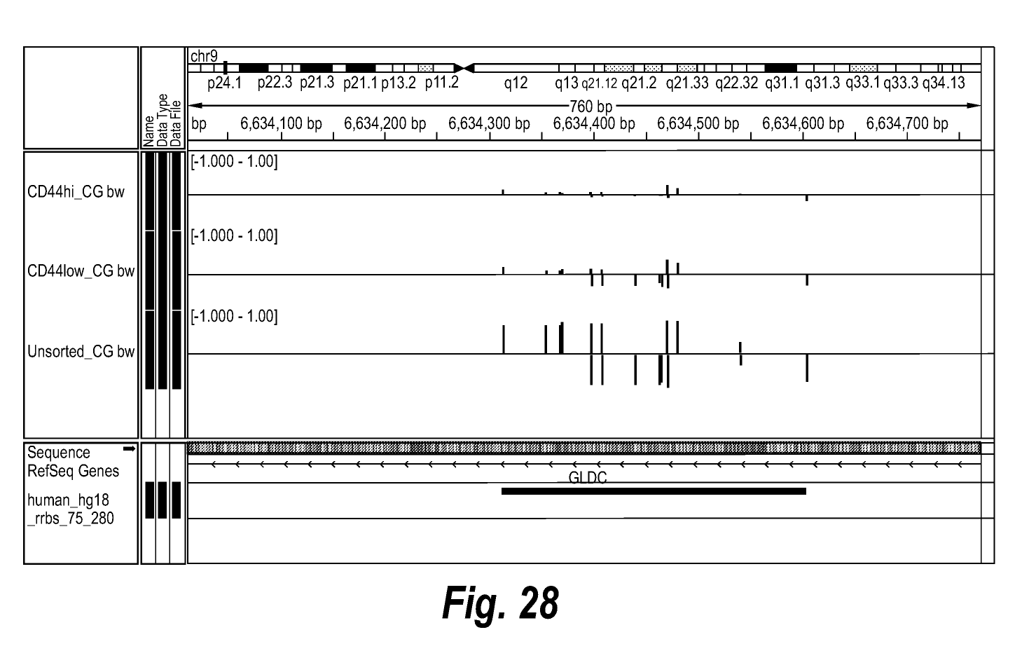

Samples (A) M-1, (B) M-2 and (C) M-3 were stained for CD44 and PI and then first gated with PI staining pattern (FIG. 27i) and then back-gated for CD44 (CD44-FITC/FL-1) and PI (FL-2A) (FIG. 27ii). The panels iii, iv and v of FIG. 27 represent histogram of cell cycle stages of CD44^{hi} and CD44^{lo} gated cells (5-10% of total cells) and un-gated total cell population respectively. FIG. 27D represents the population at different cell cycle stages G1 (M1), G2 (M2) and S (M3) stages. The CD44^{hi} cells of sample (A) M-1 shows that S and G2 phases are 15.75% and 72.61% of the gated cell population respectively (FIG. 27A iii). CD44^{lo} cells of the same sample represent 4.36% (S) and 1.81% (G2) of gated cell population (FIG. 27A iv). Thus CD44^{hi} cells were enriched 40-fold for cells in the G2 phase than the CD44^{lo} cells.

Similarly, sample (B) M-2 analysis indicated that CD44^{hi} cells in S/G2 phase were higher (12.03/32.19) than the CD44^{lo} cells (S/G2: 5.18/7.14) (FIGS. 27B iii and iv and D). In this sample, the CD44^{hi} cells were enriched for cells in S/G2 phase at 2.3/4.5 times higher than CD44^{lo} cells. In the third sample (C), M-3, cells in S/G2 phase represented 6.25/15.17 percent of the whole population, where CD44^{lo} cells in the as S/G2 represented 3.37/3.32 respectively (FIGS. 27C ii and iii and D). The gated CD44^{hi} cells at S/G2 cell cycle stages are 1.8/4.5 times higher than gated CD44^{lo} cells. In all three samples, majority of CD44^{lo} cells reside at G1 phase of the cell cycle.

The data indicate that the CD44^{hi} cells are enriched for S and G2 phase fractions more than the CD44^{lo} cells indicating slow growth, quiescence of these cells.

### DISCUSSION

In early analyses, we are unable to associate specific embryonal or polycomb markers with higher tumorigenic potentials. In the three current MPE primary samples tested, only one of the CD44^{hi} subsets expressed (M-1) the predicted pattern of candidate CSC-marker expression (lower PTEN, higher hTERT, SUZ12, EZH2, OCT4 and BMI1) than the isogenic CD44^{lo} cells. The other two samples (M-2 and M-3) were quite variable in the expression of markers on this panel. On the basis of a primary samples (n=3) that displays a highly variable expression of markers, we can speculate that it is unlikely that individual molecular markers will reliably predict the highly tumorigenic CSC-phenotype in lung cancers.

Whereas our earlier studies focused on demonstrating that candidate CSC existed in MPE by virtue of surrogate biomarker expression, this study actually associates the expression of those biomarkers with behavioral bioassays (colony formation and tumorigenesis in vivo). We clearly demonstrated that within the MPE-tumor biospecimen there are tumor cell subsets (CD44^{hi} cells) with high tumorigenic potentials. Thus, these subsets can now be characterized as having properties associated with “cancer stem cells” in three distinct surrogate measures of that property. Our data also suggest that lung CSC can be distinguished from non-CSC on the basis of several associated molecular properties and profiles. Although many additional properties are likely to emerge with prospective high throughput analyses, this report provides initial evidence of differences in cell cycle profiles, and in miRNA expression. Collectively, our studies convincingly demonstrate that behaviorally aggressive (CSC or tumor initiating cells) are present within the bulk MPE populations of lung cancer patients.

The CD44^{hi} cell subsets from different primary tumor cultures consistently formed tumors in vivo with greater efficiency (FIG. 23). However, these efficiencies and tumor growth kinetics varied quite dramatically from one sample to another. The surface labeling intensity of CD44 indicated a better proxy marker for growth kinetics. The CD44^{hi} cells from the fast growing M-1 sample displayed higher surface CD44 (MFI=28243), as compared to the CD44^{hi} cells from relatively slow growing M-2 sample (MFI=12864) (FIG. 21E). The CD44^{hi} cells from the M-1 tumor exhibited a more primitive phenotype (in terms of expected BMI, hTERT, SUZ12, EZH2, OCT-4 expression), as compared to the CD44^{hi} cells from the M-2 sample (with only higher hTERT expression) (FIG. 22E). Thus, CD44^{hi} cells from the M-1 sample were much more efficient at forming in vivo tumors than the CD44^{hi} cells from M-2 sample. These data suggest that whereas the CD44^{hi} surface phenotype may commonly predict for more efficient tumorigenesis in individual tumors, there are likely to be differences in the molecular signatures that comprise this highly tumorigenic subset.

As indicated, the main objective of the present study was to identify and extract the tumor cell subpopulations from MPE that are responsible for tumor propagation and maintenance, and to characterize their molecular signature pattern. CD44 had previously been implicated as a surface marker for CSC as indicated earlier. Our earlier studies convincingly showed that almost all the MPE primary tumor cells labeled for surface CD44 (>98%). To distinguish a behaviorally-distinct cell subset amongst a cell population that contiguously expressed the CD44 surface marker, we elected to compare tumorigenic potentials of MPE-tumor cells expressing the highest levels of surface CD44 (CD44^{hi}) with tumor cells expressing the lowest level of surface CD44)(CD44^{lo}. It was not possible to distinguish these cell subsets simply on the basis of morphology; i.e.: cells sorted on the basis of CD44^{hi} and CD44^{lo} are morphologically similar. However, the CD44^{hi} cells could be clearly distinguished by behavioral properties, such as high clonal efficiency and high spheroid formation efficiency in soft agar, the established surrogate in vitro properties of CSC like cells. Accordingly, this study identifies the CD44^{hi} surface phenotype as a marker that is associated with high tumorigenic potentials in individual lung cancers. However, the surface phenotype may not be associated with a consistent molecular profile. More importantly, this study does not predict that the surface CD44^{hi} phenotype is exclusively the cancer cell subset with higher tumorigenic potentials. Clearly, the surface CD44^{hi} phenotype is not a homogeneous population. First, the expression of the CD44 surface marker varies greatly from one tumor to another. Moreover, surface CD44 expression varies greatly between individual tumors; the tumor cells that most highly label for surface CD44 seem to possess greater competence at tumor formation.

That the CD44^{hi} subset is not a homogeneous cell subset as suggested by the co-labeling of subsets with additional candidate CSC markers (e.g.: ALDH). Only a fraction of the CD44^{hi} subpopulation can be jointly characterized as the CD44^{hi}/ALDH^{hi} surface phenotype. In order to investigate if there is a co-relationship between CD44 and other known marker of CSC/TIC we evaluated one of the most prominent markers, ALDH, for its expression pattern by immuno-histpathology in the tissues generated by CD44^{hi} implanted cells in NSG mice and primary SCC and AC of lung cancer. It is suggested that various isozymes of ALDH are expressed in different lung cancer cell lines and ALDH expression is significant for poor prognosis. ALDH, like CD44, may also have a functional role in cancer progression. Our study has shown that that only fraction of CD44^{hi} subpopulation can be jointly characterized as CD44^{h}/ALDH^{hi} surface phenotype in xenograft tissues and SCC and AC of the lung cancer.

Chromosomal abnormalities are common in cancer and in lung cancer losses and/or gains of several chromosomal regions have also been reported. We were interested to evaluate if chromosomal abnormalities are also detected in the MPE samples as has been reported for lung cancer. To evaluate these abnormalities we performed G-banded karyotype analysis and chromosome painting by using Fluorescence In Situ Hybridization (FISH). Our result indicated hyperploidy and chromosomal abnormality in all the MPE samples tested. FISH analysis of 1p36 region revealed LOH in two samples and rearrangements of both 1p/1q regions in the third MPE sample. Thus, indicating important role of region 1p36 in MPE where miR-34a maps. In this respect, data presented herein suggest that miR-34a likely represents a key etiologic factor in contributing to aggressive CSC phenotypes, and is thus a likely target for curbing the growth potentials of lung CSC in a subset of lung cancers. Specifically, a relative loss of miR-34a expression appears to contribute to aggressive behavioral features of lung CSC, and those features can be mitigated by exogenous delivery and restoration of miR34a activity.

Deletion of 1p36 in neuroblastoma has led to identification of a number of tumor suppressor genes from a 2 Mb region of this locus. These genes include TP73, CHDS, K1F1B, CAMTA1, and CASTOR (36). The p53 induced miRNA-34a also localizes to this site, and is considered to be a strong candidate tumor suppressor gene in neurobalstoma and other human cancers. Studies have shown a suppressive effect on N-myc expression in neurobalstoma (36) and CD44 in prostate cancer, supporting a role in cancer suppression. In our system the MPE derived CD44^{hi} cells exhibited low expression of miR-34a.

The three MPE samples evaluated in this study are heterogeneous. The M-1 sample was from a younger patient and had more aggressive disease (poorly differentiated NSCLC) than sample M-2 and M-3. Malignant pleural effusions are an advanced stage of disease for all subtypes of lung cancer. Our data suggested that there is considerable intra-tumoral heterogeneity at this advanced stage of progression. In addition, based on the fractional expression of individual markers, there is considerable inter-tumoral heterogeneity between clinically isolated biospecimens as well. In summary, this work substantiates the validity of our lung cancer MPE model and phenotype-based approach for the discovery of the molecular bases of functional intratumoral heterogeneity. This work extends the evidence to support our proposition that for us to effectively treat cancer, we need to approach the disease starting from a behavioral phenotype. The most efficient way for us to accomplish that task is to dissect the molecular basis of specific properties in behaviorally distinct cell subsets of individual tumors

### Example 6

### Validation of Glycine Dehydrogenase (GLDC) as a Biomarker for Lung Cancer

Aggressive tumor cell subsets reside within individual tumors. These cell subsets can be live-sorted on the basis of “cancer stem cell” (CSC) biomarkers. For example, the surface CD44^{hi} subset is reliably “more tumorigenic” (3/3 lung cancer biospecimens; by soft agar colony formation and tumor engraftment in NOD/SCID IL2γRnull mice in vivo). Compared to control (CD44^{lo} and unsorted tumor cell) subsets, the CD44^{hi} subsets also display distinct molecular differences, including many changes in DNA-methylation and microRNA (miR) expression. For example, the glycine decarboxylase (GLDC) gene is significantly hypomethlyated in the CD44^{hi} subset (Z-score of 27, change of methylation of −0.78 from baseline of “unsorted cells”). This observation is consistent with a recent report that identifies GLDC as a key molecular target that distinguishes tumorigenic CD166^{hi} from non-tumorigenic CD166^{lo} lung cancer cell subsets.

The data indicate that the CD44^{hi} and CD44^{lo} subsets also display highly significant differences in GLDC-gene methylation. The data suggest that 1) observed differences in GLDC-mRNA expression in the CD166^{hi} and CD166^{lo} subsets was attributable to differences in gene methylation; and 2) that despite us using a different surface marker (CD44) to extract “CSC”, it is possible that that the molecular signatures converge when they are directly associated with a specific behavioral phenotype (e.g.: high tumorigenic potential).

GLDC is also differentially methylated despite using a different surface marker (CD44) for cell separation. A representative screenshot of differences in the GLDC-gene methylation is depicted in FIG. 21. Using the unsorted set as the baseline, differences in DNA-methylation (represented by the relative amplitude of the bars on a sequenced GLDC-gene fragment) in the CD44^{hi} versus CD44^{lo} subsets are clear. The screenshot suggests that the GLDC gene is more methylated in the unsorted tumor mix than in the sorted CD44^{hi} and CD44^{lo} subsets.

The CD44^{hi} and CD44^{lo} subsets display differences in GLDC-gene methylation by direct sequencing. The CD44^{hi} subset is very significantly different (Z-score of 27, change of methylation of −0.78 from baseline of “unsorted cells”) in the whole genome methylation analysis.

### Example 7

### Identification of Sox2 as a Candidate Biomarker for Live-Sorting Lung Cancer Stem Cells from MPE Cultures

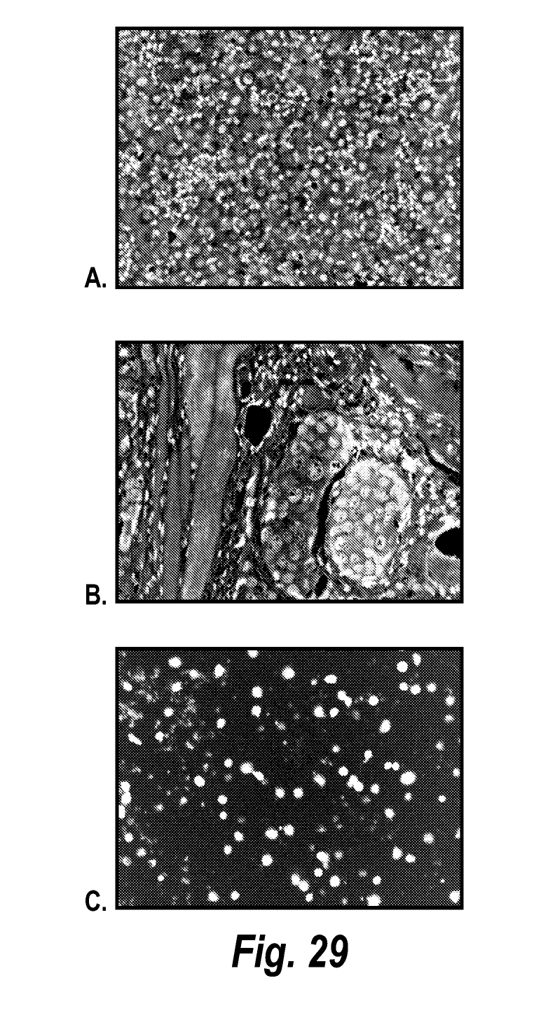

FIGS. 29A and B show primary lung cancer cells labeled for Sox2. FIG. 29C shows the same labeling for thr cell line H520. This shows that one can transcriptionally sort fractions of cancer cells that preferentially highly label for primordial transcription factors (such as TTF1/Nkx2.1; Sox2, and Grhl2) in clinical biospecimens.

### REFERENCES

1. Siegel R, Naishadham D, Jemal A. Cancer statistics, 2013. CA Cancer J Clin. 2013 January; 63(1):11-30. Epub 2013 January 17.
2. Naruke T, Tsuchiya R, Kondo H, Asamura H, Nakayama H. Implications of staging in lung cancer. Chest. 1997 October; 112(4 Suppl):2425-2485.
3. Sugiura S, Ando Y, Minami H, Ando M, Sakai S, Shimokata K. Prognostic value of pleural effusion in patients with non-small cell lung cancer. Clin Cancer Res. 1997 January; 3(1):47-50.
4. Mott F E, Sharma N, Ashley P. Malignant pleural effusion in non-small cell lung cancer—time for a stage revision? Chest. 2001 January; 119(1):317-8.
5. Basak S K, Veena M S, Oh S, Huang G, Srivatsan E, Huang M, et al. The malignant pleural effusion as a model to investigate intratumoral heterogeneity in lung cancer. PLoS One. 2009 Jun. 12; 4(6).
6. Neumeister V, Agarwal S, Bordeaux J, Camp R L, Rimm D L. In situ identification of putative cancer stem cells by multiplexing ALDH1, CD44, and cytokeratin identifies breast cancer patients with poor prognosis. Am J Pathol. 2010 May; 176(5):2131-8.
7. Joshua B, Kaplan M J, Doweck I, Pai R, Weissman I L, Prince M E, et al. Frequency of cells expressing CD44, a head and neck cancer stem cell marker: correlation with tumor aggressiveness. Head Neck. 2012 January; 34(1):42-9.
8. Su J, Xu X H, Huang Q, Lu M Q, Li D J, Xue F, Yi F, Ren J H, Wu Y P. Identification of cancer stem-like CD44+ cells in human nasopharyngeal carcinoma cell line. Arch Med Res. 2011 January; 42(1):15-21.
9. Shi C, Tian R, Wang M, Wang X, Jiang J, Zhang Z, et al. CD44+CD133+ population exhibits cancer stem cell-like characteristics in human gallbladder carcinoma. Cancer Biol Ther. 2010 Dec. 1; 10(11):1182-90
10. Wei H J, Yin T, Zhu Z, Shi P F, Tian Y, Wang C Y. Expression of CD44, CD24 and ESA in pancreatic adenocarcinoma cell lines varies with local microenvironment. Hepatobiliary Pancreat Dis Int. 2011 August; 10(4):428-34.
11. Patrawala L, Calhoun T, Schneider-Broussard R, Li H, Bhatia B, et al. Highly purified CD44+ prostate cancer cells from xenograft human tumors are enriched in tumorigenic and metastatic progenitor cells. Oncogene. 2006 Mar. 16; 25(12):1696-708.
12. Patrawala L, Calhoun-Davis T, Schneider-Broussard R, Tang D G. Hierarchical organization of prostate cancer cells in xenograft tumors: the CD44+alpha2beta1+ cell population is enriched in tumor-initiating cells. Cancer Res. 2007 Jul. 15; 67(14):6796-805. Erratum in: Cancer Res. 2007 Sep. 15; 67(18):8973.
13. Hurt E M, Kawasaki B T, Klarmann G J, Thomas S B, Farrar W L. CD44+CD24(−) prostate cells are early cancer progenitor/stem cells that provide a model for patients with poor prognosis. Br J Cancer. 2008 Feb. 26; 98(4):756-65.
14. Eaton C L, Colombel M, van der Pluijm G, Cecchini M, Wetterwald A, Lippitt J, et al. Evaluation of the frequency of putative prostate cancer stem cells in primary and metastatic prostate cancer. Prostate. 2010 Jun. 1; 70(8):875-82.
15. Liu C, Kelnar K, Liu B, Chen X, Calhoun-Davis T, Li H, et al. The microRNA miR-34a inhibits prostate cancer stem cells and metastasis by directly repressing CD44. Nat Med. 2011 February; 17(2):211-5. Epub 2011 January 16.
16. Leung E L, Fiscus R R, Tung J W, Tin V P, Cheng L C, Sihoe A D, et al. Non-small cell lung cancer cells expressing CD44 are enriched for stem cell-like properties. PLoS One. 2010 Nov. 19; 5(11):e14062.
17. Takanami I, Takeuchi K, Naruke M. Expression and prognostic value of the standard CD44 protein in pulmonary adenocarcinoma. Oncol Rep. 2000 September-October; 7(5):1065-7.
18. Travis W D, Brambilla E, Riely G J. New pathologic classification of lung cancer: relevance for clinical practice and clinical trials. J Clin Oncol. 2013 Mar. 10; 31(8):992-1001. Epub 2013 February 11. Review.
19. Gallardo E, Navarro A, Viliolas N, Marrades R M, Diaz T, Gel B, et al. miR-34a as a prognostic marker of relapse in surgically resected non-small-cell lung cancer. Carcinogenesis. 2009 November; 30(11):1903-9.
20. Srivatsan E S, Chakrabarti R, Zainabadi K, Pack S D, Benyamini P, Mendonca M S, Yang P K, Kang K, Motamedi D, Sawicki M P, Zhuang Z, Jesudasan R A, Bengtsson U, Sun C, Roe B A, Stanbridge E J, Wilczynski S P, Redpath J L. Localization of deletion to a 300 Kb interval of chromosome 11q13 in cervical cancer. Oncogene. 2002 Aug. 15; 21(36):5631-42.
21. Phelps R M, Johnson B E, Ihde D C, Gazdar A F, Carbone D P, McClintock P R, Linnoila R I, Matthews M J, Bunn P A Jr, Carney D, Minna J D, Mulshine J L. NCI-Navy Medical Oncology Branch cell line data base. J Cell Biochem Suppl. 1996; 24:32-91.
22. Qin, M., Chen, S., Yu, T., Escuadro, B., Sharma, S., and Batra, R. K. 2003. Coxsackievirus adenovirus receptor expression predicts the efficiency of adenoviral gene transfer into non-small cell lung cancer xenografts. Clin Cancer Res 9:4992-4999.
23. Veena, M. S., Qin, M., Andersson, A., Sharma, S., and Batra, R. K. 2009. CAR mediates efficient tumor engraftment of mesenchymal type lung cancer cells. Lab Invest 89:875-886.
24. Basak S K, Harui A, Stolina M, Sharma S, Mitani K, Dubinett S M, et al. Increased dendritic cell number and function following continuous in vivo infusion of granulocyte macrophage-colony-stimulating factor and interleukin-4. Blood. 2002 Apr. 15; 99(8):2869-79.
25. Srivatsan E S, Bengtsson U, Manickam P, Benyamini P, Chandrasekharappa S C, Sun C, et al. Interstitial deletion of 11q13 sequences in HeLa cells. Genes Chromosomes Cancer. 2000 October; 29(2):157-65.
26. Winokur S T, Bengtsson U, Vargas J C, Wasmuth J J, Altherr M R, Weiffenbach B, et al. The evolutionary distribution and structural organization of the homeobox-containing repeat D4Z4 indicates a functional role for the ancestral copy in the FSHD region. Hum Mol Genet. 1996 October; 5(10):1567-75. Erratum in: Hum Mol Genet. 1997 March; 6(3):502.
27. Miki J, Rhim J S. Prostate cell cultures as in vitro models for the study of normal stem cells and cancer stem cells. Prostate Cancer Prostatic Dis. 2008; 11(1):32-9.
28. Simpson-Abelson M R, Sonnenberg G F, Takita H, Yokota S J, Conway T F Jr, Kelleher R J Jr, Shultz L D, Barcos M, Bankert R B. Long-term engraftment and expansion of tumor-derived memory T cells following the implantation of non-disrupted pieces of human lung tumor into NOD-scid IL2Rgamma(null) mice. J Immunol. 2008 May 15; 180(10):7009-18. PubMed PMID: 18453623.
29. Gee H E, Buffa F M, Camps C, Ramachandran A, Leek R, Taylor M, et al. The small-nucleolar RNAs commonly used for microRNA normalisation correlate with tumour pathology and prognosis. Br J Cancer. 2011 Mar. 29; 104(7):1168-77.
30. Harper L J, Costea D E, Gammon L, Fazil B, Biddle A, Mackenzie I C. Normal and malignant epithelial cells with stem-like properties have an extended G2 cell cycle phase that is associated with apoptotic resistance. BMC Cancer. 2010 Apr. 28; 10:166.
31. Zhang Y, Wei J, Wang H, Xue X, An Y, Tang D, et al. Epithelial mesenchymal transition correlates with CD24+CD44+ and CD133+ cells in pancreatic cancer. Oncol Rep. 2012 May; 27(5):1599-605.
32. Sullivan J P, Spinola M, Dodge M, Raso M G, Behrens C, Gao B, et al. Aldehyde dehydrogenase activity selects for lung adenocarcinoma stem cells dependent on notch signaling. Cancer Res. 2010 Dec. 1; 70(23):9937-48. Epub 2010 Nov. 30.
33. Moreb J S, Baker H V, Chang L J, Amaya M, Lopez M C, Ostmark B, et al. ALDH isozymes downregulation affects cell growth, cell motility and gene expression in lung cancer cells. Mol Cancer. 2008 Nov. 24; 7:87.
34. Wang Y C, Yo Y T, Lee H Y, Liao Y P, Chao T K, Su P H, et al. ALDH1-bright epithelial ovarian cancer cells are associated with CD44 expression, drug resistance, and poor clinical outcome. Am J Pathol. 2012 March; 180(3): 1159-69.
35. Mitsuuchi Y, Testa J R. Cytogenetics and molecular genetics of lung cancer. Am J Med Genet. 2002 Oct. 30; 115(3):183-8.
36. Cole K A, Attiyeh E F, Mosse Y P, Laquaglia M J, Diskin S J, Brodeur G M, et al. A functional screen identifies miR-34a as a candidate neuroblastoma tumor suppressor gene. Mol Cancer Res. 2008 May; 6(5):735-42.
37. Nalls D, Tang S N, Rodova M, Srivastava R K, Shankar S. Targeting epigenetic regulation of miR-34a for treatment of pancreatic cancer by inhibition of pancreatic cancer stem cells. PLoS One. 2011; 6(8):e24099. Epub 2011 Aug. 31.
38. de Antonellis P, Medaglia C, Cusanelli E, Andolfo I, Liguori L, De Vita G, et al. MiR-34a targeting of Notch ligand delta like 1 impairs CD15+/CD133+ tumor-propagating cells and supports neural differentiation in medulloblastoma. PLoS One. 2011; 6(9):e24584.
39. Guessous F, Zhang Y, Kofman A, Catania A, Li Y, Schiff D, et al. microRNA-34a is tumor suppressive in brain tumors and glioma stem cells. Cell Cycle. 2010 Mar. 15; 9(6):1031-6. Epub 2010 Mar. 15.
40. Wiggins J F, Ruffino L, Kelnar K, Omotola M, Patrawala L, Brown D, et al. Development of a lung cancer therapeutic based on the tumor suppressor microRNA-34. Cancer Res. 2010 Jul. 15; 70(14):5923-30. Epub 2010 Jun. 22.
41. Trang P, Wiggins J F, Daige C L, Cho C, Omotola M, Brown D, et al. Systemic delivery of tumor suppressor microRNA mimics using a neutral lipid emulsion inhibits lung tumors in mice. Mol Ther. 2011 June; 19(6):1116-22. Epub 2011 Mar. 22.
42. Batra R K, Warburton D. On the derivation and clinical implications of “driver” mutations in lung cancer. Am J Respir Crit Care Med. 2010 Jul. 1; 182(1):4-5.

1. A method of assessing the tumorigenic potential of individual tumor populations in a population of cancer cells comprising:
a) isolating a sample from the subject comprising the population of cancer cells;
b) separating individual tumor populations in the population of cancer cells from each other based on differential RNA or protein expression; and
c) assessing the tumorigenic potential of the separated individual tumor populations.

2. The method of claim 1, wherein the separation is performed using fluorescence activated cell sorting (FACS).

3. The method of claim 1, wherein the assessment of tumorigenic potential is performed in vitro.

4. The method of claim 3, wherein the in vitro assessment of tumorigenic potential is performed using a soft agar test.

5. The method of claim 2, wherein the assessment of tumorigenic potential is performed in vivo.

6. The method of claim 5, wherein the in vivo assessment of tumorigenic potential is performed using immunocompromised mice.

7. The method of claim 1, further comprising obtaining a single cell suspension of the population of cancer cells after step a) and prior to step b).

8. The method of claim 1, wherein the population of cancer cells is isolated from a single tumor in the subject.

9. The method of claim 1, wherein the tumor population comprises cells that are CD24+, CD44hi, Nkx2.1 (TTF-1)+, SOX-2+, Kras+, p53+, Sca1+, miR34alo or CD133+.

10. The method of claim 9, wherein the cancer is lung cancer.

11. The method of claim 10, wherein the sample is a malignant pleural effusion (MPE).

12. A method of screening for an effective therapeutic for treatment of a cancer comprising:
a) separating individual tumor populations in a population of cancer cells from the cancer to be treated from each other based on differential RNA or protein expression; and
b) assessing the tumorigenic potential of the separated individual tumor populations;
c) screening the individual tumor populations with tumorigenic potential for susceptibility to various cancer therapeutics;
wherein, if the screened cancer therapeutic reduces the proliferative capacity of the individual tumor populations with tumorigenic potential then the screened cancer therapeutic is an effective therapeutic for treatment of the cancer in the subject.

13. The method of claim 12, wherein the separation is performed using fluorescence activated cell sorting (FACS).

14. The method of claim 12, wherein the assessment of tumorigenic potential is performed in vitro.

15. The method of claim 14, wherein the in vitro assessment of tumorigenic potential is performed using a soft agar test.

16. The method of claim 12, wherein the assessment of tumorigenic potential is performed in vivo.

17. The method of claim 16, wherein the in vivo assessment of tumorigenic potential is performed using immunocompromised mice.

18. The method of claim 12, further comprising obtaining a single cell suspension of the population of cancer cells after step a) and prior to step b).

19. The method of claim 12, wherein the population of cancer cells is isolated from a single tumor in the subject.

20. The method of claim 12, wherein the tumor population comprises cells that are CD24+, CD166+; CD44hi, Nkx2.1 (TTF-1)+, SOX-2+, mutated Kras+, mutated or lost p53+, miR34alo or CD133+.

21. The method of claim 20, wherein the cancer is lung cancer.

22. The method of claim 21, wherein the sample is a malignant pleural effusion (MPE).

23. A method of treating cancer in a subject in need thereof comprising:
a) isolating a sample from the subject comprising cancer cells;
b) separating individual tumor populations from each other;
c) assessing the tumorigenic potential of the individual tumor populations;
d) screening the individual tumor populations with high tumorigenic potential for susceptibility to various cancer treatments; and
e) administering to the subject a cancer treatment that one or more of the individual tumor populations with high tumorigenic potential is susceptible to,
thereby treating cancer in the subject in need thereof.

24. The method of claim 23, wherein the separation is performed using fluorescence activated cell sorting (FACS).

25. The method of claim 23, wherein the assessment of tumorigenic potential is performed in vitro.

26. The method of claim 25, wherein the in vitro assessment of tumorigenic potential is performed using a soft agar test.

27. The method of claim 23, wherein the assessment of tumorigenic potential is performed in vivo.

28. The method of claim 27, wherein the in vivo assessment of tumorigenic potential is performed using immunocompromised mice.

29. The method of claim 23, further comprising obtaining a single cell suspension of the population of cancer cells after step a) and prior to step b).

30. The method of claim 23, wherein the population of cancer cells is isolated from a single tumor in the subject.

31. The method of claim 23, wherein the tumor population comprises cells that are CD24+, CD44hi, Nkx2.1 (TTF-1)+, SOX-2+, Kras+, p53+, Sca1+, miR34alo or CD133+.

32. The method of claim 31, wherein the cancer is lung cancer.

33. The method of claim 32, wherein the sample is a malignant pleural effusion (MPE).

34. A method of screening for a biomarker of an individual tumor population with tumorigenic potential comprising:
a) separating individual tumor populations in a population of cancer cells from the cancer to be treated from each other based on differential RNA or protein expression; and
b) assessing the tumorigenic potential of the separated individual tumor populations; and
wherein, if the individual tumor population has tumorigenic potential then the RNA or protein that was used to separate the individual tumor population based on differential expression is a biomarker of an individual tumor population with tumorigenic potential.

35. The method of claim 34, wherein the separation is performed using fluorescence activated cell sorting (FACS).

36. The method of claim 34, wherein the assessment of tumorigenic potential is performed in vitro.

37. The method of claim 36, wherein the in vitro assessment of tumorigenic potential is performed using a soft agar test.

38. The method of claim 34, wherein the assessment of tumorigenic potential is performed in vivo.

39. The method of claim 38, wherein the in vivo assessment of tumorigenic potential is performed using immunocompromised mice.

40. The method of claim 34, further comprising obtaining a single cell suspension of the population of cancer cells after step a) and prior to step b).

41. The method of claim 34, wherein the population of cancer cells is isolated from a single tumor in the subject.

42. The method of claim 34, wherein the tumor population comprises cells that CD24+, CD44hi, Nkx2.1 (TTF-1)+, SOX-2+, Kras+, p53+, Sca1+, miR34alo or CD133+.

43. The method of claim 42, wherein the cancer is lung cancer.

44. The method of claim 43, wherein the sample is a malignant pleural effusion (MPE).

45. A cell line wherein the cell line is derived from lung cancer cells and wherein the cell line over expresses a protein selected from the group consisting of CD24, CD44, Nkx2.1 (TTF-1), SOX-2, Kras, p53, Sca1 and CD133.

46. The cell line of claim 45, wherein the cell line derived from lung cells is selected from the group consisting of NCI-H1373, NCI-H1395, SK-LU-1, HCC2935, HCC4006, HCC827, NCI-H1581, NCI-H23, Human, NCI-H522, NCI-H1435, NCI-H1563, NCI-H1651, NCI-H1734, NCI-H1793, NCI-H1838, NCI-H1975, NCI-H2073, NCI-H2085, NCI-H2228 and NCI-H2342.

47. The cell line of claim 45, wherein the cell line comprises an expression vector wherein the expression vector expresses a protein selected from the group consisting of CD24, CD44, Nkx2.1 (TTF-1), SOX-2, Kras, p53, Sca1 and CD133 in the cell line.

48. A cell line wherein the cell line is derived from lung cancer cells and wherein the cell line under expresses miR34a.

49. The cell line of claim 48, wherein the cell line derived from lung cells is selected from the group consisting of NCI-H1373, NCI-H1395, SK-LU-1, HCC2935, HCC4006, HCC827, NCI-H1581, NCI-H23, Human, NCI-H522, NCI-H1435, NCI-H1563, NCI-H1651, NCI-H1734, NCI-H1793, NCI-H1838, NCI-H1975, NCI-H2073, NCI-H2085, NCI-H2228 and NCI-H2342.

50. The cell line of claim 48, wherein the cell line comprises a vector wherein the vector knocks down the expression of miR34a in the cell line.
